SEQUENCE LISTING (108 sequences)

SEQ ID NO: 1 - protein, 351 aa, Artificial Sequence
  Immature LukA consensus
  misc_feature (63)..(63): Xaa can be any naturally occurring amino acid
MKNKKRVLIASSLSCAILLLSAATTQANSAHKDSQDQNKKEHVDKSQQKEKRNVTNKDKNSTXPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 2 - protein, 351 aa, Artificial Sequence
  Immature LukA
MKNKKRVLIASSLSCAILLLSAATTQANSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 3 - protein, 351 aa, Artificial Sequence
  Immature LukA
MKNKKRVFIASSLSCALLLLSAANTEANSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 4 - protein, 350 aa, Artificial Sequence
  Immature LukA
MKNKKRVLIASSLSCAILLLSAATTQANSAHKDSQDQNKKEHVDKSQQKEKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 5 - protein, 350 aa, Artificial Sequence
  Immature LukA
MKNKKRVLIASSLSCAILLLSAATTQANSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYTDNKSFREG

SEQ ID NO: 6 - protein, 350 aa, Artificial Sequence
  Immature LukA
MKNKKRVLIASSLSCAILLLSAATTQANSAHKDSQDQNKKEHVDKSQQKEKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 7 - protein, 351 aa, Artificial Sequence
  Immature LukA
MKNKKRVLIASSLSCAILLLSAATTQANSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 8 - protein, 351 aa, Artificial Sequence
  Immature LukA
MKNKKRVLIASSLSCAILLLSAATTQANSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQKYDTIAIGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 9 - protein, 351 aa, Artificial Sequence; identical to SEQ ID NO: 2
  Immature LukA

SEQ ID NO: 10 - protein, 351 aa, Artificial Sequence
  Immature LukA
MKNKKRVFIASSLSCALLLLSAANTEANSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKEG

SEQ ID NO: 11 - protein, 351 aa, Artificial Sequence
  Immature LukA
MKNKKRVFIASSLSCALLLLSAANTEANSANKDSQDQTKKEHVDKAQQKEKRNVNDKDKNTPGPDDIGKNGKVTKRTETVYDEKTNILQNLQFDFIDDPTYDKNVLLVKKQGSIHSNLKFESHKEENNSSWLKYPSEYHVDFQVKSNRKTEILDQLPKNKISTAKVDSTFSYNSGGKFDSVKGVGRTSSNSYSKTISYNQQNYDTIASGKNNNWHVHWSVVANDLKYGGEVKNRNDDFLFYRNTRLSTVENPELSFASKYRYPALVRSGFNPEFLTYLSNEKSNEKTQFEVTYTRNQDVLKNKPGIHYAPPILEKNKDGQRLIVTYEVDWKNKTVKVIDKYSDENKPYKEG

SEQ ID NO: 12 - protein, 351 aa, Artificial Sequence
  Immature LukA
MKNKKRVFIASSLSCVLLLLSAANTEANSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 13 - protein, 351 aa, Artificial Sequence
  Immature LukA
MKNKKRVFIASSLSCALLLLSAANTEANSANKDSQDQTKKEHVDKAQQKEKRNVNDKDKNTPGPDDIGKNGKVTKRTETVYDEKTNILQNLQFDFIDDPTYDKNILLVKKQGSIHSNLKFESHKEENNSSWLKYPSEYHVDFQVKRNRKTEILDQLPKNKISTAKVDSTFSYNSGGKFDSVKGVGRTSSNSYSKTISYNQQNYDTIASGKNNNWHVHWSVVANDLKYGGEVKNRNDEFLFYRTTRLSTVENPELSFASKYRYPALVRSGFNPEFLTYLSNEKSNEKTQFEVTYTRNQDILKNKPGIHYAPPILEKNKDGQRLIVTYEVDWKNKTVKVIDKYSDDNKPYKEG

SEQ ID NO: 14 - protein, 351 aa, Artificial Sequence
  Immature LukA
MKNKKRVLIASSLSCAILLLSAATTQANSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPSILEKNKDGQRLIVTYEVDWKNKTVKVVDKYSDDNKPYKEG

SEQ ID NO: 15 - protein, 324 aa, Artificial Sequence
  Mature LukA consensus
  misc_feature (36)..(36): Xaa can be any naturally occurring amino acid
NSAHKDSQDQNKKEHVDKSQQKEKRNVTNKDKNSTXPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 16 - protein, 324 aa, Artificial Sequence
  Mature LukA
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 17 - protein, 324 aa, Artificial Sequence
  Mature LukA
NSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 18 - protein, 323 aa, Artificial Sequence
  Mature LukA
NSAHKDSQDQNKKEHVDKSQQKEKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 19 - protein, 323 aa, Artificial Sequence
  Mature LukA
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYTDNKSFREG

SEQ ID NO: 20 - protein, 323 aa, Artificial Sequence
  Mature LukA
NSAHKDSQDQNKKEHVDKSQQKEKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 21 - protein, 324 aa, Artificial Sequence
  Mature LukA
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 22 - protein, 324 aa, Artificial Sequence
  Mature LukA
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQKYDTIAIGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 23 - protein, 324 aa, Artificial Sequence; identical to SEQ ID NO: 16
  Mature LukA

SEQ ID NO: 24 - protein, 324 aa, Artificial Sequence
  Mature LukA
NSANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKTNDKTRFEVTYTRNQDILKNKPGIHYGQPILEQNKDGQRFIVVYEVDWKNKTVKVVEKYSDQNKPYKEG

SEQ ID NO: 25 - protein, 324 aa, Artificial Sequence
  Mature LukA
NSANKDSQDQTKKEHVDKAQQKEKRNVNDKDKNTPGPDDIGKNGKVTKRTETVYDEKTNILQNLQFDFIDDPTYDKNVLLVKKQGSIHSNLKFESHKEENNSSWLKYPSEYHVDFQVKSNRKTEILDQLPKNKISTAKVDSTFSYNSGGKFDSVKGVGRTSSNSYSKTISYNQQNYDTIASGKNNNWHVHWSVVANDLKYGGEVKNRNDDFLFYRNTRLSTVENPELSFASKYRYPALVRSGFNPEFLTYLSNEKSNEKTQFEVTYTRNQDVLKNKPGIHYAPPILEKNKDGQRLIVTYEVDWKNKTVKVIDKYSDENKPYKEG

SEQ ID NO: 26 - protein, 324 aa, Artificial Sequence; identical to SEQ ID NO: 17
  Mature LukA

SEQ ID NO: 27 - protein, 324 aa, Artificial Sequence
  Mature LukA
NSANKDSQDQTKKEHVDKAQQKEKRNVNDKDKNTPGPDDIGKNGKVTKRTETVYDEKTNILQNLQFDFIDDPTYDKNILLVKKQGSIHSNLKFESHKEENNSSWLKYPSEYHVDFQVKRNRKTEILDQLPKNKISTAKVDSTFSYNSGGKFDSVKGVGRTSSNSYSKTISYNQQNYDTIASGKNNNWHVHWSVVANDLKYGGEVKNRNDEFLFYRTTRLSTVENPELSFASKYRYPALVRSGFNPEFLTYLSNEKSNEKTQFEVTYTRNQDILKNKPGIHYAPPILEKNKDGQRLIVTYEVDWKNKTVKVIDKYSDDNKPYKEG

SEQ ID NO: 28 - protein, 324 aa, Artificial Sequence
  Mature LukA
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPSILEKNKDGQRLIVTYEVDWKNKTVKVVDKYSDDNKPYKEG

SEQ ID NO: 29 - protein, 338 aa, Artificial Sequence
  Immature LukB consensus
MIKQLCKNITICSLALSTALTVFPATSYAKINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 30 - protein, 339 aa, Artificial Sequence
  Immature LukB
MIKQVCKNITICSLALSTALTVFPASSYAEIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTFNDKEKK

SEQ ID NO: 31 - protein, 290 aa, Artificial Sequence
  Immature LukB
MYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 32 - protein, 338 aa, Artificial Sequence
  Immature LukB
MIKQVCKNITICSLALSTALTIFPASSYAKINSEIKQVSEKNLDGETKMYTRTATTSDSQKNITQSLQFNFLTEKNYDKETVFIKAKGTIGSGLRILEPNGYWNSTLRWPGSYSVSIQNVDDNNNTNVTDFAPKNQDESREVKYTYGYKTGGDFSINQGGLTGNITKESNYSETISYQQPSYRTLIDQPTTNKGVAWKVEAHLINNMGHDHTRQLTNDSDDRVKSEIFSLTRNGNLWAKDNFTPKNKMPVTVSEGFNPEFLAVMSHDKKDEGKSKFVVHYKRSMDEFKIDWNKHGFWGYWSGENHVDKKEEKLSALYEVDWKTHNVKFIKVLNDKEKK

SEQ ID NO: 33 - protein, 339 aa, Artificial Sequence
  Immature LukB
MIKQVCKNITICSLALSTALTVFPASSYAEIKSKITTVSEKNLDGDTKMYTRTATTSDTEKKISQSLQFNFLTEPNYDKETVFIKAKGTIGSGLKILNPNGYWNSTLTWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 34 - protein, 338 aa, Artificial Sequence
  Immature LukB
MIKQLCKNITICTLALSTTFTVLPATSFAKINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDEKEEKLSALYEVDWKTHNVKFVKVLNDNEKK

SEQ ID NO: 35 - protein, 339 aa, Artificial Sequence
  Immature LukB
MIKQVCKNITICSLALSTALTVFPASSYAEIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 36 - protein, 338 aa, Artificial Sequence
  Immature LukB
MIKQLYKNITICSLAISTALTVFPATSYAKINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHDVKFVKVLNDNEKK

SEQ ID NO: 37 - protein, 338 aa, Artificial Sequence
  Immature LukB
MIKQLYKNITICTLALSTTFTVLPATSYAKINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 38 - protein, 338 aa, Artificial Sequence
  Immature LukB
MIKQLYKNITICSLTISTALTVFPATSYAKINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLPGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHDVKFVKVLNDNEKK

SEQ ID NO: 39 - protein, 338 aa, Artificial Sequence
  Immature LukB
MIKQLCKNITICTLALSTTFTVLPATSFAKINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 40 - protein, 338 aa, Artificial Sequence
  Immature LukB
MIKQLCKNITICTLALSTTFTVLPATSFAKINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 41 - protein, 338 aa, Artificial Sequence; identical to SEQ ID NO: 40
  Immature LukB

SEQ ID NO: 42 - protein, 309 aa, Artificial Sequence
  Mature LukB consensus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 43 - protein, 310 aa, Artificial Sequence
  Mature LukB
EIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTFNDKEKK

SEQ ID NO: 44 - protein, 309 aa, Artificial Sequence
  Mature LukB
KINSEIKQVSEKNLDGETKMYTRTATTSDSQKNITQSLQFNFLTEKNYDKETVFIKAKGTIGSGLRILEPNGYWNSTLRWPGSYSVSIQNVDDNNNTNVTDFAPKNQDESREVKYTYGYKTGGDFSINQGGLTGNITKESNYSETISYQQPSYRTLIDQPTTNKGVAWKVEAHLINNMGHDHTRQLTNDSDDRVKSEIFSLTRNGNLWAKDNFTPKNKMPVTVSEGFNPEFLAVMSHDKKDEGKSKFVVHYKRSMDEFKIDWNKHGFWGYWSGENHVDKKEEKLSALYEVDWKTHNVKFIKVLNDKEKK

SEQ ID NO: 45 - protein, 310 aa, Artificial Sequence
  Mature LukB
EIKSKITTVSEKNLDGDTKMYTRTATTSDTEKKISQSLQFNFLTEPNYDKETVFIKAKGTIGSGLKILNPNGYWNSTLTWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 46 - protein, 309 aa, Artificial Sequence
  Mature LukB
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDEKEEKLSALYEVDWKTHNVKFVKVLNDNEKK

SEQ ID NO: 47 - protein, 310 aa, Artificial Sequence
  Mature LukB
EIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 48 - protein, 309 aa, Artificial Sequence
  Mature LukB
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHDVKFVKVLNDNEKK

SEQ ID NO: 49 - protein, 309 aa, Artificial Sequence
  Mature LukB
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 50 - protein, 309 aa, Artificial Sequence
  Mature LukB
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLPGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHDVKFVKVLNDNEKK

SEQ ID NO: 51 - protein, 309 aa, Artificial Sequence
  Mature LukB
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 52 - protein, 309 aa, Artificial Sequence
  Mature LukB
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 53 - protein, 309 aa, Artificial Sequence; identical to SEQ ID NO: 52
  Mature LukB

SEQ ID NO: 54 - protein, 291 aa, Artificial Sequence
  Full length SpAkkaa
AQHDEAKKNAFYQVLNMPNLNADQRNGFIQSLKAAPSQSANVLGEAQKLNDSQAPKADAQQNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 55 - protein, 52 aa, Artificial Sequence
  SpA A domain
NNFNKEQQNAFYEILNMPNLNEEQRNGFIQSLKDDPSQSANLLSEAKKLNES

SEQ ID NO: 56 - protein, 52 aa, Artificial Sequence
  SpA B domain
NKFNKEQQNAFYEILHLPNLNEEQRNGFIQSLKDDPSQSANLLAEAKKLNDA

SEQ ID NO: 57 - protein, 52 aa, Artificial Sequence
  SpA C domain
NKFNKEQQNAFYEILHLPNLTEEQRNGFIQSLKDDPSVSKEILAEAKKLNDA

SEQ ID NO: 58 - protein, 54 aa, Artificial Sequence
  SpA D domain
QQNNFNKDQQSAFYEILNMPNLNEAQRNGFIQSLKDDPSQSTNVLGEAKKLNES

SEQ ID NO: 59 - protein, 51 aa, Artificial Sequence
  SpA E domain
QHDEAQQNAFYQVLNMPNLNADQRNGFIQSLKDDPSQSANVLGEAQKLNDS

SEQ ID NO: 60 - protein, 291 aa, Artificial Sequence
  SpA S33E_UoC
AQHDEAKKNAFYQVLNMPNLNADQRNGFIQELKDDPSQSANVLGEAQKLNDSQAPKADAQQNNFNKDKKSAFYEILNMPNLNEAQRNGFIQELKDDPSQSTNVLGEAKKLNESQAPKADNNFNKEKKNAFYEILNMPNLNEEQRNGFIQELKDDPSQSANLLSEAKKLNESQAPKADNKFNKEKKNAFYEILHLPNLNEEQRNGFIQELKDDPSQSANLLAEAKKLNDAQAPKADNKFNKEKKNAFYEILHLPNLTEEQRNGFIQELKDDPSVSKEILAEAKKLNDAQAPK

SEQ ID NO: 61 - protein, 291 aa, Artificial Sequence
  SpA S33T
AQHDEAKKNAFYQVLNMPNLNADQRNGFIQTLKDDPSQSANVLGEAQKLNDSQAPKADAQQNNFNKDKKSAFYEILNMPNLNEAQRNGFIQTLKDDPSQSTNVLGEAKKLNESQAPKADNNFNKEKKNAFYEILNMPNLNEEQRNGFIQTLKDDPSQSANLLSEAKKLNESQAPKADNKFNKEKKNAFYEILHLPNLNEEQRNGFIQTLKDDPSQSANLLAEAKKLNDAQAPKADNKFNKEKKNAFYEILHLPNLTEEQRNGFIQTLKDDPSVSKEILAEAKKLNDAQAPK

SEQ ID NO: 62 - protein, 52 aa, Artificial Sequence
  SpA S33E A domain
NNFNKEKKNAFYEILNMPNLNEEQRNGFIQELKDDPSQSANLLSEAKKLNES

SEQ ID NO: 63 - protein, 52 aa, Artificial Sequence
  SpA S33E B domain
NKFNKEKKNAFYEILHLPNLNEEQRNGFIQELKDDPSQSANLLAEAKKLNDA

SEQ ID NO: 64 - protein, 52 aa, Artificial Sequence
  SpA S33E C domain
NKFNKEKKNAFYEILHLPNLTEEQRNGFIQELKDDPSVSKEILAEAKKLNDA

SEQ ID NO: 65 - protein, 51 aa, Artificial Sequence
  SpA S33E E domain
QHDEAKKNAFYQVLNMPNLNADQRNGFIQELKDDPSQSANVLGEAQKLNDS

SEQ ID NO: 66 - protein, 54 aa, Artificial Sequence
  SpA S33E D domain
QQNNFNKDKKSAFYEILNMPNLNEAQRNGFIQELKDDPSQSTNVLGEAKKLNES

SEQ ID NO: 67 - protein, 52 aa, Artificial Sequence
  SpA S33T A domain
NNFNKEKKNAFYEILNMPNLNEEQRNGFIQTLKDDPSQSANLLSEAKKLNES

SEQ ID NO: 68 - protein, 52 aa, Artificial Sequence
  SpA S33T B domain
NKFNKEKKNAFYEILHLPNLNEEQRNGFIQTLKDDPSQSANLLAEAKKLNDA

SEQ ID NO: 69 - protein, 52 aa, Artificial Sequence
  SpA S33T C domain
NKFNKEKKNAFYEILHLPNLTEEQRNGFIQTLKDDPSVSKEILAEAKKLNDA

SEQ ID NO: 70 - protein, 51 aa, Artificial Sequence
  SpA S33T E domain
QHDEAKKNAFYQVLNMPNLNADQRNGFIQTLKDDPSQSANVLGEAQKLNDS

SEQ ID NO: 71 - protein, 54 aa, Artificial Sequence
  SpA S33T D domain
QQNNFNKDKKSAFYEILNMPNLNEAQRNGFIQTLKDDPSQSTNVLGEAKKLNES

SEQ ID NO: 72 - protein, 516 aa, Artificial Sequence
  SpA Long
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 73 - protein, 291 aa, Artificial Sequence
  SpAXX
  MISC_FEATURE (7)..(8): Any amino acid except Gln
  MISC_FEATURE (34)..(35): Any amino acid except Asp
  MISC_FEATURE (68)..(69): Any amino acid except Gln
  MISC_FEATURE (95)..(96): Any amino acid except Asp
  MISC_FEATURE (126)..(127)
  MISC_FEATURE (153)..(154): Any amino acid except Asp
  MISC_FEATURE (184)..(185): Any amino acid except Gln
  MISC_FEATURE (211)..(212): Any amino acid except Asp
  MISC_FEATURE (242)..(243): Any amino acid except Gln
  MISC_FEATURE (269)..(270): Any amino acid except Asp
AQHDEAXXNAFYQVLNMPNLNADQRNGFIQSLKXXPSQSANVLGEAQKLNDSQAPKADAQQNNFNKDXXSAFYEILNMPNLNEAQRNGFIQSLKXXPSQSTNVLGEAKKLNESQAPKADNNFNKEXXNAFYEILNMPNLNEEQRNGFIQSLKXXPSQSANLLSEAKKLNESQAPKADNKFNKEXXNAFYEILHLPNLNEEQRNGFIQSLKXXPSQSANLLAEAKKLNDAQAPKADNKFNKEXXNAFYEILHLPNLTEEQRNGFIQSLKXXPSVSKEILAEAKKLNDAQAPK

SEQ ID NO: 74 - protein, 291 aa, Artificial Sequence; identical to SEQ ID NO: 54
  SpAkkAA

SEQ ID NO: 75 - protein, 291 aa, Artificial Sequence
  SpAkR
  MISC_FEATURE (60)..(61): Any amino acid except Gln
AQHDEAKKNAFYQVLNMPNLNADQRNGFIQSLKAAPSQSANVLGEAQKLNDSQAPKADAXXNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 76 - protein, 291 aa, Artificial Sequence
  SpAkR
AQHDEAKKNAFYQVLNMPNLNADQRNGFIQSLKAAPSQSANVLGEAQKLNDSQAPKADAKRNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 77 - protein, 292 aa, Artificial Sequence
  SpAkR
MAQHDEAKKNAFYQVLNMPNLNADQRNGFIQSLKAAPSQSANVLGEAQKLNDSQAPKADAKRNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 78 - protein, 291 aa, Artificial Sequence
  SpAkR
  MISC_FEATURE (7)..(8): Any amino acid except Gln
  MISC_FEATURE (34)..(35): Any amino acid except Asp
  MISC_FEATURE (60)..(61): Any amino acid except Gln
  MISC_FEATURE (68)..(69): Any amino acid except Gln
  MISC_FEATURE (95)..(96): Any amino acid except Asp
  MISC_FEATURE (126)..(127): Any amino acid except Gln
  MISC_FEATURE (153)..(154): Any amino acid except Asp
  MISC_FEATURE (184)..(185): Any amino acid except Gln
  MISC_FEATURE (211)..(212): Any amino acid except Asp
  MISC_FEATURE (242)..(243): Any amino acid except Gln
  MISC_FEATURE (269)..(270): Any amino acid except Asp
AQHDEAXXNAFYQVLNMPNLNADQRNGFIQSLKXXPSQSANVLGEAQKLNDSQAPKADAXXNNFNKDXXSAFYEILNMPNLNEAQRNGFIQSLKXXPSQSTNVLGEAKKLNESQAPKADNNFNKEXXNAFYEILNMPNLNEEQRNGFIQSLKXXPSQSANLLSEAKKLNESQAPKADNKFNKEXXNAFYEILHLPNLNEEQRNGFIQSLKXXPSQSANLLAEAKKLNDAQAPKADNKFNKEXXNAFYEILHLPNLTEEQRNGFIQSLKXXPSVSKEILAEAKKLNDAQAPK

SEQ ID NO: 79 - protein, 67 aa, Artificial Sequence
  SpAkR E domain
  MISC_FEATURE (7)..(8): Any amino acid except Gln
  MISC_FEATURE (34)..(35): Any amino acid except Asp
  MISC_FEATURE (60)..(61): Any amino acid except Gln
AQHDEAXXNAFYQVLNMPNLNADQRNGFIQSLKXXPSQSANVLGEAQKLNDSQAPKADAXXNNFNKD

SEQ ID NO: 80 - protein, 67 aa, Artificial Sequence
  SpAkR E domain
  MISC_FEATURE (7)..(8): Any amino acid except Gln
  MISC_FEATURE (34)..(35): Any amino acid except Asp
AQHDEAXXNAFYQVLNMPNLNADQRNGFIQSLKXXPSQSANVLGEAQKLNDSQAPKADAKRNNFNKD

SEQ ID NO: 81 - protein, 67 aa, Artificial Sequence
  SpAkR E domain
AQHDEAKKNAFYQVLNMPNLNADQRNGFIQSLKAAPSQSANVLGEAQKLNDSQAPKADAKRNNFNKD

SEQ ID NO: 82 - protein, 68 aa, Artificial Sequence
  E domain of SpAkR in examples
MAQHDEAKKNAFYQVLNMPNLNADQRNGFIQSLKAAPSQSANVLGEAQKLNDSQAPKADAKRNNFNKD

SEQ ID NO: 83 - protein, 67 aa, Artificial Sequence
  SpA E domain
AQHDEAQQNAFYQVLNMPNLNADQRNGFIQSLKDDPSQSANVLGEAQKLNDSQAPKADAQQNNFNKD

SEQ ID NO: 84 - protein, 516 aa, Artificial Sequence
  SpA 252
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDGNKPGKEDNKKPGKEDGNKPGKEDNKKPGKEDGNKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 85 - protein, 296 aa, Artificial Sequence
  SpA5 (KKAA)
GPLGSAQHDEAKKNAFYQVLNMPNLNADQRNGFIQSLKAAPSQSANVLGEAQKLNDSQAPKADAKKNKFNKDQQSAFYEILNMPNLNEEQRNGFIQSLKAAPSQSTNVLGEAKKLNESQAPKADNNFNKEKKNAFYEILNMPNLNEEQRNGFIQSLKAAPSQSANLLAEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 86 - protein, 296 aa, Artificial Sequence
  SpA5 (RRAA)
GPLGSAQHDEARRNAFYQVLNMPNLNADQRNGFIQSLKAAPSQSANVLGEAQKLNDSQAPKADARRNKFNKDQQSAFYEILNMPNLNEEQRNGFIQSLKAAPSQSTNVLGEAKKLNESQAPKADNNFNKERRNAFYEILNMPNLNEEQRNGFIQSLKAAPSQSANLLAEAKKLNESQAPKADNKFNKERRNAFYEILHLPNLNEEQRNGFIQSLKAAPSQSANLLAEAKKLNDAQAPKADNKFNKERRNAFYEILHLPNLTEEQRNGFIQSLKAAPSVSKEILAEAKKLNDAQAPK

SEQ ID NO: 87 - protein, 296 aa, Artificial Sequence
  SpA5 (KKVV)
GPLGSAQHDEAKKNAFYQVLNMPNLNADQRNGFIQSLKVVPSQSANVLGEAQKLNDSQAPKADAKKNKFNKDQQSAFYEILNMPNLNEEQRNGFIQSLKVVPSQSTNVLGEAKKLNESQAPKADNNFNKEKKNAFYEILNMPNLNEEQRNGFIQSLKVVPSQSANLLAEAKKLNESQAPKADNKFNKEKKNAFYEILHLPNLNEEQRNGFIQSLKVVPSQSANLLAEAKKLNDAQAPKADNKFNKEKKNAFYEILHLPNLTEEQRNGFIQSLKVVPSVSKEILAEAKKLNDAQAPK

SEQ ID NO: 88 - protein, 296 aa, Artificial Sequence
  SpA5 (RRVV)
GPLGSAQHDEARRNAFYQVLNMPNLNADQRNGFIQSLKVVPSQSANVLGEAQKLNDSQAPKADARRNKFNKDQQSAFYEILNMPNLNEEQRNGFIQSLKVVPSQSTNVLGEAKKLNESQAPKADNNFNKERRNAFYEILNMPNLNEEQRNGFIQSLKVVPSQSANLLAEAKKLNESQAPKADNKFNKERRNAFYEILHLPNLNEEQRNGFIQSLKVVPSQSANLLAEAKKLNDAQAPKADNKFNKERRNAFYEILHLPNLTEEQRNGFIQSLKVVPSVSKEILAEAKKLNDAQAPK

SEQ ID NO: 89 - DNA, 19 bp, Artificial Sequence
  1095 F primer
agacgatccttcggtgagc

SEQ ID NO: 90 - DNA, 22 bp, Artificial Sequence
  1517R primer
gcttttgcaatgtcatttactg

SEQ ID NO: 91 - DNA, 23 bp, Artificial Sequence
  arc-up primer
ttgattcaccagcgcgtattgtc

SEQ ID NO: 92 - DNA, 21 bp, Artificial Sequence
  arc-dn primer
aggtatctgcttcaatcagcg

SEQ ID NO: 93 - DNA, 23 bp, Artificial Sequence
  aro-up primer
atcggaaatcctatttcacattc

SEQ ID NO: 94 - DNA, 23 bp, Artificial Sequence
  aro-dn primer
ggtgttgtattaataacgatatc

SEQ ID NO: 95 - DNA, 22 bp, Artificial Sequence
  glp-up primer
ctaggaactgcaatcttaatcc

SEQ ID NO: 96 - DNA, 23 bp, Artificial Sequence
  glp-dn primer
tggtaaaatcgcatgtccaattc

SEQ ID NO: 97 - DNA, 20 bp, Artificial Sequence
  gmk-dn primer
atcgttttatcgggaccatc

SEQ ID NO: 98 - DNA, 23 bp, Artificial Sequence
  gmk-dn primer
tcattaactacaacgtaatcgta

SEQ ID NO: 99 - DNA, 23 bp, Artificial Sequence
  pta-up primer
gttaaaatcgtattacctgaagg

SEQ ID NO: 100 - DNA, 23 bp, Artificial Sequence
  pta-dn primer
gacccttttgttgaaaagcttaa

SEQ ID NO: 101 - DNA, 22 bp, Artificial Sequence
  tpi-up primer
tcgttcattctgaacgtcgtga

SEQ ID NO: 102 - DNA, 23 bp, Artificial Sequence
  tpi-dn primer
tttgcaccttctaacaattgtac

SEQ ID NO: 103 - DNA, 23 bp, Artificial Sequence
  yqi-up primer
cagcatacaggacacctattggc

SEQ ID NO: 104 - DNA, 23 bp, Artificial Sequence
  yqi-dn primer
cgttgaggaatcgatactggaac

SEQ ID NO: 105 - DNA, 57 bp, Artificial Sequence
  ext1F primer
ggggaccactttgtacaagaaagctgggtcatttaagaagattgtttcagatttatg

SEQ ID NO: 106 - DNA, 51 bp, Artificial Sequence
  ext1F primer
atttgtaaagtcatcataatataacgaattatgtattgcaatactaaaatc

SEQ ID NO: 107 - DNA, 46 bp, Artificial Sequence
  ext2F primer
cgtcgcgaactataataaaaacaaacaatacacaacgatagatatc

SEQ ID NO: 108 - DNA, 56 bp, Artificial Sequence
  ext2R primer
ggggacaagtttgtacaaaaaagcaggcaacgaacgcctaaagaaattgtctttgc

(19) United States
(12) Patent Application Publication
(10) Pub. No.: US 2022/0362368 A1
(43) Pub. Date: Nov. 17, 2022

(54) STAPHYLOCOCCUS PEPTIDES AND METHODS OF USE

(71) Applicants: Janssen Vaccines & Prevention B.V., Leiden (NL); The University of Chicago, Chicago, IL (US)

(72) Inventors: Jan Theunis POOLMAN, Leiden (NL); Olaf SCHNEEWIND, Chicago, IL (US); Dominique MISSIAKAS, Chicago, IL (US); Yan SUN, Chicago, IL (US); Hwan Keun KIM, Chicago, IL (US); Miaomiao SHI, Chicago, IL (US); Xinhai CHEN, Chicago, IL (US); Jeffrey A. FERNANDEZ, Beachwood, NJ (US)

(21) Appl. No.: 17/764,434

(22) PCT Filed: Oct. 2, 2020
(86) PCT No.: PCT/US2020/054047

§ 371 Date: Mar. 28, 2022

Related U.S. Application Data
(60) Provisional application No. 62/909,458, filed on Oct. 2, 2019.
(60) Provisional application No. 62/909,473, filed on Oct. 2, 2019.

(51) Int. Cl. A61K 39/085; A61P 31/04

(52) U.S. Cl. CPC A61K 39/085; A61P 31/04

(57) ABSTRACT

Provided herein are immunogenic compositions comprising a Staphylococcus aureus protein A (SpA) variant and a mutant staphylococcal leukocidin subunit polypeptide comprising a LukA polypeptide, a LukB polypeptide, and/or a LukAB dimer polypeptide, wherein the LukA polypeptide, the LukB polypeptide, and/or the LukAB dimer polypeptide have one or more amino acid substitutions, deletions, or a combination thereof.

### CROSS REFERENCE TO RELATED APPLICATIONS

This application claims priority to U.S. Provisional Application No. 62/909,458, filed Oct. 2, 2019; and U.S. Provisional Application No. 62/909,473, filed Oct. 2, 2019. Each disclosure is incorporated herein by reference in its entirety.

### FIELD OF THE INVENTION

This invention generally relates to the fields of immunology, microbiology, and biotechnology. More particularly, to the field and use of peptides to generate an immune response. Specifically, the invention relates to the use of Staphylococcus peptides and methods of using the same to induce an immune response and/or treat or prevent a Staphylococcus infection. REFERENCE TO SEQUENCE LISTING SUBMITTED ELECTRONICALLY

This application contains a sequence listing, which is submitted electronically via EFS-Web as an ASCII formatted sequence listing with a file name “004852.150WO1 Sequence Listing” and a creation date of Sep. 22, 2020 and having a size of 211 kb. The sequence listing submitted via EFS-Web is part of the specification and is herein incorporated by reference in its entirety.

### BACKGROUND OF THE INVENTION

Staphylococcus aureus represents the most common pathogen found in skin and soft tissue infections and also the predominant pathogen in surgical wounds. S. aureus also is a lead cause of bloodstream infections. The surgical site infections (SSI) are a consequence of surgical incision and tend to develop between 15 days and 3 years post-surgery and more typically, 30 days after an operation or within one year if an implant was placed. In the United States, S. aureus is responsible for 11% of all hospital-acquired infections, including 14% of SSI, and 14% of bloodstream infections (Kallen et al., JAMA 304(6):641-7 (2010); Johnson et al., J. Antimicrob. Chemother. 67(4):802-9 (2012); Laupland et al., Clin. Microbiol. Infect. 19(5):465-71 (2013); and Monaco et al., Current Topics Microbiol. Immunol. 409:21-56 (2017).

Often bacteremia is a consequence of SSI originating from one or multiple abscesses. Acute bacterial skin and skin structure infections (ABSSSI) can also lead to bacteremia. In the industrialized world, the population incidence of S. aureus associated bacteremia (SAB) ranges from 10 to 30 per 100,000 person-years (Johnson et al., Antimicrob. Chemother. 67(4):802-9 (2012)). Age seems to be a very powerful determinant of SAB incidence. High rates in the first year of life are followed by the low incidence throughout young adulthood, and a gradual rise in incidence with advancing age. For example, the incidence of SAB is >100 per 100,000 person-years among subject >70 years of age but it is significantly lower in younger people (Laupland et al., Clin. Microbiol. Infect. 19(5):465-71 (2013)). To note, bacteremia among the elderly is associated with high mortality. Infection with methicillin-resistant S aureus (MRSA) carries a worse prognosis than infection with methicillin-sensitive S. aureus in the elderly. Hence, both total mortality and mortality directly attributable to SAB are more than twice as likely in older patients (Kaasch et al., J. Infection 68(3):242-51 (2014)). The importance of MRSA as causative agent of BSIs is as great in the community as in the hospital setting, while MSSA is gaining in importance as a cause of community-onset BSIs, which is highly contributing to the overall rise in the incidence of S. aureus infections (Kaasch et al., J. Infection 68(3):242-51 (2014)). Hence, both MRSA and MSSA are therefore important contributors to severe SSI and SAB disease.

Staphylococcus infections are typically treated with antibiotics, with penicillin being the drug of choice and vancomycin being used for methicillin resistant isolates. The percentage of Staphylococcal strains exhibiting wide-spectrum resistant to antibiotics has increased, posing a threat to effective antimicrobial therapy. In addition, the recent appearance of vancomycin-resistant Staphylococcus aureus strains has created fear that MRSA strains for which no effective therapy is available are starting to emerge and spread.

An alternative approach to antibiotics in the treatment of Staphylococcal infections has been the use of antibodies against Staphylococcal antigens in passive immunotherapy. Examples of this passive immunotherapy involves administration of polyclonal antisera (WO2000/015238; WO2000/012132) as well as treatment with monoclonal antibodies against lipoteichoic acid (WO1998/057994).

The first generation of vaccines targeted against Staphylococcus or against the exoproteins produced by Staphylococcus strains have met with limited success, and, thus, there remains a need to develop additional compositions for the treatment and/or prevention of Staphylococcus infections.

### BRIEF SUMMARY OF THE INVENTION

S. aureus has developed numerous immune escape mechanisms and secretes various virulence factors that enable the bacterium to survive within the host. Inactivation and neutralization of the virulence factors involved in antibody mediated opsonophagocytosis is the pivotal immune mechanism that controls staphylococcal infectious diseases.

Staphylococcus protein A (SpA), a surface protein, is one key virulence factor that displays at least two functions. First, cell wall-anchored SpA on the bacterial surface binds to the Fcγ-domain of IgG and disables the effector functions of antibodies. Antibodies are bound unspecifically “upside down,” thereby protecting staphylococci from opsonophagocytic killing (OPK) by host immune cells and preventing proper clearance. Second, SpA serves as a key immune evasion determinant that prevents the development of protective immunity during S. aureus colonization and infection. During colonization and invasive disease, released SpA crosslinks VH3 clonal B cell receptors and triggers the secretion of antibodies not specific to S. aureus that are unable to recognize staphylococcal determinants as antigens. This B cell superantigen activity (i.e., the VH3-binding activity of released SpA) is responsible for preventing the development of protective immunity against S. aureus during colonization or invasive disease. The use of a SpA variant as vaccine antigen that has lost its immunoglobulin binding activity induces SpA specific antibodies that (1) neutralize its ability to bind IgG via Fcγ, (2) neutralizes its ability to bind IgG via VH3-idiotype heavy chains and enables anti-staphylococcal immunity to develop, and (3) induces opsonophagocytic clearance via surface bound SpA.

Staphylococcal leukocidin LukAB is another virulence factor with a different mode of action. LukAB is a secreted toxin that, upon binding to phagocytic cells, assembles into a pore, inserts into the membrane, and lyses the host cell. This allows S. aureus to escape the attack from neutrophils and escape clearance by the host. Antibodies induced by immunization with a LukAB toxoid will neutralize LukAB toxin activity resulting in surviving phagocytic cells that can clear S. aureus.

A vaccine containing the two antigens SpA and LukAB will therefore induce antibodies that neutralize two S. aureus virulence factors and prevent two independent key escape mechanisms of S. aureus, and will allow antibody mediated opsonophagocytosis to be effective.

It was found herein that following vaccination with the vaccine combinations of the present invention, vaccine antibodies (i.e., that were elicited following vaccination) generated against both SpA variant polypeptides and mutant LukAB polypeptides provided synergistic protection and efficient S. aureus killing due to a dual-mechanism. On the one hand, the neutralization of the SpA molecule prevented the upside-down binding of antibodies (IgG Fc binding) and prevented B-cell dysregulation by disrupting SpA binding to VH3. On the other hand, the neutralization of the LukAB toxins prevented the lysing of phagocytic cells by LukAB, and, therefore, allowed for human neutrophils to remain functional and capable of eliminating S. aureus by opsonophagocytosis. The antibody response was productive, as the antibodies bound the respective target, and the phagocytic cells were capable of killing, i.e., there was a clear and additive synergistic effect of neutralizing both SpA and LukAB.

Provided herein are immunogenic compositions comprising (a) a Staphylococcus aureus protein A (SpA) variant polypeptide, wherein the SpA variant polypeptide comprises at least one SpA A, B, C, D, or E domain; and (b) a mutant staphylococcal leukocidin subunit polypeptide comprising (i) a mutant LukA polypeptide, (ii) a mutant LukB polypeptide, and/or (iii) a mutant LukAB dimer polypeptide, wherein (i), (ii), and/or (iii) have one or more amino acid substitutions, deletions, or a combination thereof, such that the ability of the mutant LukA, LukB, and/or LukAB polypeptides to form pores in the surface of eukaryotic cells is disrupted, thereby reducing the toxicity of the mutant LukA and/or LukB polypeptide or the mutant LukAB dimer polypeptide relative to the corresponding wild-type LukA and/or LukB polypeptide or LukAB dimer polypeptide.

In certain embodiments, the SpA variant polypeptide has at least one amino acid substitution that disrupts Fc binding and at least a second amino acid substitution that disrupts V_{H}3 binding.

In certain embodiments, the SpA variant polypeptide comprises a SpA D domain and has an amino acid sequence having at least 90% identity to the amino acid sequence of SEQ ID NO:58. The SpA variant polypeptide can, for example, have one or more amino acid substitutions at amino acid position 9 or 10 of SEQ ID NO:58.

In certain embodiments, the SpA variant polypeptide further comprises a SpA E, A, B, or C domain. The SpA variant polypeptide can, for example, comprise a SpA E, A, B, and C domain and have an amino acid sequence having at least 90% identity to the amino acid sequence of SEQ ID NO:54. In certain embodiments, each SpA E, A, B, and C domain has one or more amino acid substitutions at positions corresponding to amino acid positions 9 and 10 of SEQ ID NO:58. The amino acid substitution is a lysine residue for a glutamine residue.

In certain embodiments, the immunogenic compositions comprising: (a) a Staphylococcus aureus protein A (SpA) variant polypeptide, wherein said SpA variant polypeptide comprises at least one SpA A, B, C, D, or E domain, and wherein said domain has (i) lysine substitutions for glutamine residues in each of the at least one SpA A, B, C, D, or E domains corresponding to positions 9 and 10 in the SpA D domain and (ii) a glutamate substitution in each of the at least one SpA A, B, C, D, or E domains corresponding to position 33 in the SpA D domain, wherein the polypeptide does not, relative to a negative control, detectably crosslink IgG and IgE in blood or activate basophils; and (b) a mutant staphylococcal leukocidin subunit polypeptide comprising (1) a mutant LukA polypeptide, (2) a mutant Luk B polypeptide, and/or (3) a mutant LukAB dimer polypeptide, wherein (1), (2), and/or (3) have one or more amino acid substitutions, deletions, or a combination thereof; such that the ability of the mutant LukA, LukB, and/or LukAB polypeptides to form pores in the surface of eukaryotic cells is disrupted, thereby reducing the toxicity of the mutant LukA and/or LukB polypeptide or the mutant LukAB dimer polypeptide relative to the corresponding wild-type LukA and/or LukB polypeptide or LukAB dimer polypeptide. In certain embodiments, the SpA domain D comprises SEQ ID NO:58.

In certain embodiments, the SpA variant polypeptide has a reduced K_{A} binding affinity for V_{H}3 from human IgG as compared to a SpA variant polypeptide (SpA_{KKAA}) comprising lysine substitutions for glutamine residues in each SpA A-E domain corresponding to positions 9 and 10 in the SpA D domain and alanine substitutions for aspartic acid residues in each SpA A-E domain corresponding to positions 36 and 37 of SpA D domain. The SpA variant polypeptide can, for example, have a K_{A} binding affinity for V_{H}3 from human IgG that is reduced by at least 2-fold as compared to SpA_{KKAA}. The SpA variant polypeptide can, for example, have a K_{A} binding affinity for V_{H}3 from human IgG of less than 1×10⁵ M⁻¹. In certain embodiments, SpA_{KKAA} comprises SEQ ID NO:54.

In certain embodiments, the SpA variant polypeptide does not have substitutions in any of the SpA A, B, C, D, or E domains corresponding to amino acid positions 36 and 37 in the SpA D domain. In certain embodiments, the only substitutions in the SpA variant polypeptide are (i) and (ii).

In certain embodiments, the mutant LukA polypeptide comprises an amino acid sequence having at least 80%, at least 90%, at least 91%, at least 92%, at least 93%, at least 94%, at least 95%, at least 96%, at least 97%, at least 98%, or at least 99% sequence identity to any one of SEQ ID NOs:1-28. The mutant LukA polypeptide can, for example, comprise a deletion of the amino acid residues corresponding to amino acid positions 342-351 of any one of SEQ ID NOs:1-14 and at amino acid positions 315-324 of any one of SEQ ID NOs:15-28.

In certain embodiments, the mutant LukB polypeptide comprises an amino acid sequence having at least 80%, at least 90%, at least 91%, at least 92%, at least 93%, at least 94%, at least 95%, at least 96%, at least 97%, at least 98%, or at least 99% sequence identity to any of SEQ ID NO:29-53.

In certain embodiments, the mutant LukAB dimer polypeptide comprises a mutant LukA polypeptide having a deletion of the amino acid residues corresponding to positions 315-324 of SEQ ID NO:16; and a mutant LukB polypeptide comprising the amino acid sequence of SEQ ID NO:53.

In certain embodiments, the immunogenic composition further comprises an adjuvant. The adjuvant can, for example, comprise saponins, e.g., QS21. The adjuvant can, for example, comprise a TLR4 agonist, e.g., the TLR4 agonist is lipid A or an analog or derivative thereof. The TLR4 agonist can, for example, comprise MPL, 3D-MPL, RC529, GLA, SLA, E6020, PET-lipid A, PHAD, 3D-PHAD, 3D-(6-acyl)-PHAD, ONO4007, or OM-174. The TLR4 agonist can, for example, be GLA.

In certain embodiments, the immunogenic composition further comprises at least one staphylococcal antigen or immunogenic fragment thereof selected form the group consisting of CP5, CP8, Eap, Ebh, Emp, EsaB, EsaC, EsxA, EsxB, EsxAB(fusion), SdrC, SdrD, SdrE, IsdA, IsdB, IsdC, ClfA, ClfB, Coa, Hla, mHla, MntC, rTSST-1, rTSST-1v, TSST-1, SasF, vWbp, vWh vitronectin binding protein, Aaa, Aap, Ant, autolysin glucosaminidase, autolysin amidase, Can, collagen binding protein, Csa1A, EFB, Elastin binding protein, EPB, FbpA, fibrinogen binding protein, Fibronectin binding protein, FhuD, FhuD2, FnbA, FnbB, GehD, HarA, HBP, Immunodominant ABC transporter, IsaA/PisA, laminin receptor, Lipase GehD, MAP, Mg2+ transporter, MHC II analog, MRPII, NPase, RNA III activating protein (RAP), SasA, SasB, SasC, SasD, SasK, SBI, SdrF, SdrG, SdrH, SEA exotoxins, SEB exotoxins, mSEB, SitC, Ni ABC transporter, SitC/MntC/saliva binding protein, SsaA, SSP-1, SSP-2, Spa5, SpA_{KKAA}, SpAkR, Sta006, Sta011, PVL, LukED and Hlg.

Also provided are one or more isolated nucleic acids encoding a Staphylococcus aureus protein A (SpA) variant polypeptide and a mutant Luk A polypeptide, a mutant Luk B polypeptide, or a mutant LukAB dimer polypeptide of the invention. Also provided are vectors comprising the isolated nucleic acids of the invention. Also provided are isolated host cells comprising the vectors of the invention.

Also provided are methods of inducing an immune response in a subject in need thereof. The methods comprise administering to the subject in need thereof an effective amount of an immunogenic composition described herein. The immunogenic composition can, for example, further comprise an adjuvant.

Also provided are methods for treating or preventing a Staphylococcus infection in a subject in need thereof. The methods comprise administering to the subject in need thereof an effective amount of an immunogenic composition described herein. Methods also include a method for decolonization or for preventing colonization or recolonization of Staphylococcus bacteria in a subject and a method for eliciting an immune response against a staphylococcus bacterium in a subject by administering compositions of the disclosure. The immunogenic composition can, for example, further comprise an adjuvant. The Staphylococcus infection can further be defined as a Staphylococcus aureus infection. In some embodiments, the Staphylococcus infection is a methicillin resistant Staphylococcus aureus (MRSA) infection.

In certain embodiments, including method, composition, and polypeptide embodiments, the Staphylococcus bacteria can, for example, comprise the WU1 or JSNZ strain of Staphylococcus aureus. In some embodiments, the Staphylococcus bacteria comprises the ST88 isolate. In other examples, S. aureus isolates may belong to sequence types (ST) 5, ST8, ST22, ST30, ST45, ST398, and their respective S. aureus clonal complexes (CC) associated with human and animal invasive disroders.

In certain embodiments, the subject or patient described herein, such as the human patient is a pediatric patient. A pediatric patient is one that is defined as less than 18 years old. In some embodiments, the patient is at least or at most 1, 2, 3, 4, 5, 6, 7, 8, 9, 10, 11, 12, 13, 14, 15, 16, 17, 18, 19, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 85, or 90 years old (or any range derivable therein). In some embodiments, the pediatric patient is 2 years old or less. In some embodiments, the pediatric patient is less than 1 year old. In some embodiments, the pediatric patient is less than 6 months old. In some embodiments, the pediatric patient is 2 months old or less. In some embodiments, the human patient is 65 years old or older. In some embodiments, the human patient is a health care worker. In some embodiments, the patient is one that will receive a surgical procedure.

In certain embodiments, the patient the isolated polypeptide of composition is administered in four doses and wherein the interval between doses is at least four weeks. In some embodiments, the isolated polypeptide is given in 4 doses or in exactly 4 doses. In some embodiments, the isolated polypeptide or composition is given in at least, at most, or exactly 1, 2, 3, 4, 5, 6, 7, or 8 doses. In some embodiments, the first dose is administered at 6-8 weeks of age. In some embodiments, all four doses are administered at or before 2 years of age. In some embodiments, the polypeptide or composition is to be administered as a four-dose series at 2, 4, 6, and 12-15 months of age. Dose 1 may be given as early as 6 weeks of age. The interval between dosing may be about 4 to 8 weeks. In some embodiments, the fourth dose is administered at approximately 12-15 months of age, and at least 2 months after the third dose.

Further aspects relate to a method of making a composition, the method comprising mixing a SpA variant polypeptide of the disclosure and a mutant staphylococcal leukocidin subunit polypeptide of the disclosure in a pharmaceutical composition.

In certain embodiments, the immunogenic composition is administered in combination with a second therapy. The second therapy can, for example, be at least one antibiotic. The at least one antibiotic can, for example, be selected from the group consisting of streptomycin, ciprofloxacin, doxycycline, gentamycin, chloramphenicol, trimethoprim, sulfamethoxazole, ampicillin, tetracycline, and combinations thereof.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1E. Staphylococcus aureus ST88 isolate WU1, a mouse pathogen. (FIG. 1A) Domain structure and sequence homology of the vwb gene products from S. aureus WU1 and S. aureus Newman, a human clinical isolate. The percent amino acid (a.a.) identity of vWbp for its signal peptide (S), D1 and D2 domains (responsible for binding and activation of host prothrombin), linker (white box) and C-terminal fibrinogen binding domain (C) is displayed. (FIG. 1B) Immunoblot of S. aureus whole culture samples of strains Newman (WT, wild-type) as well as its Δcoa, Δvwb, Δcoa-vwb, and ΔclfA variants, strains WU1, JSNZ, USA300 LAC and its Δvwb variant were analyzed for the production of vWbp (avWbp), Coa (aCoa), Hla (aHla), and ClfA (αClfA) using polyclonal rabbit antibodies. (FIG. 1C) Polyclonal antibodies against the vWbp-C domain identify the vWbp allelic variant from strains JSNZ and WU1 as well as vWbp from strain USA300 LAC. (FIG. 1D-1E) Agglutination of Syto-9 stained S. aureus strains in human (FIG. 1D) or mouse (FIG. 1E) plasma was measured as average size and standard error of the means of clumped bacteria in 12 fields of microscopic view and statistical significance was assessed in pairwise comparison with WT using two-way ANOVA with Sidak multiple comparison tests. ****, p<0.0001.

FIGS. 2A-2B. S. aureus WU1 persistently colonizes the nasopharynx of C57BL/6 mice. Cohorts of C57BL/6 mice (n=10) mice were inoculated intra-nasally with 1×10⁸ CFU of indicated S. aureus WU1 or PBS control and were swabbed in the throat weekly to enumerate the bacterial load. Each dot indicates the number of CFU per mouse. The median and standard deviation for each group of animals on a given day are indicated by the horizontal line and error bar.

FIGS. 3A-3B. S. aureus WU1 expression of staphylococcal protein A (SpA) is required for persistent colonization of C57BL/6 mice. (FIG. 3A) Immunoblot of S. aureus lysates derived from strains USA300 LAC, Newman, WU1, the Δspa variant of WU1 without and with a plasmid for spa expression (pspa) were probed with SpA- (αSpA) and sortase A-specific antibodies (αSrtA). (FIG. 3B) Cohorts of C57BL/6 mice (n=10) were inoculated intra-nasally with 1×10⁸ CFU of S. aureus WU1 or its Δspa variant and the oropharynx of animals was swabbed in weekly intervals to enumerate the bacterial load. Each dot indicates the number of CFU per mouse. The median and standard deviation for each group of animals on a given day are indicated by the horizontal line and error bar. Bacterial colonization data sets were analyzed with two-way ANOVA and Sidak multiple comparison tests; statistically significant differences (*** p=0.0003; **** p<0.0001) between the two groups of animals are indicated by asterisks.

FIG. 4. Immunization of C57BL/6 mice with SpA_{KKAA} promotes decolonization of S. aureus WU1. C57BL/6 mice were immunized with 50 μg of purified recombinant SpA_{KKAA} emulsified with CFA or PBS-mock in CFA, and boosted after 11 days with 50 μg of recombinant SpA_{KKAA} emulsified with IFA or PBS-mock in IFA. On day 0 of the colonization experiment, cohorts of C57BL/6 mice (n=10) mice were inoculated intra-nasally with 1×10⁸ CFU of S. aureus WU1. The oropharynx of animals was swabbed in weekly intervals to enumerate the bacterial load. Each dot indicates the number of CFU per mouse. The median and standard deviation for each group of animals on a given day are indicated by the horizontal line and error bar. Bacterial colonization data sets were analyzed with two-way ANOVA and Sidak multiple comparison tests; statistically significant differences (*p<0.05; **p<0.01) between the two groups of animals are indicated by asterisks.

FIG. 5. Immunization of BALB/c mice with SpA_{KKAA} promotes decolonization of S. aureus WU1. BALB/c mice were immunized with 50 μg of purified recombinant SpA_{KKAA} emulsified with CFA or PBS-mock in CFA, and boosted after 11 days with 50 μg of recombinant SpA_{KKAA} emulsified with IFA or PBS-mock in IFA. On day 0 of the colonization experiment, cohorts BALB/c mice (n=10) mice were inoculated intra-nasally with 1×10⁸ CFU of S. aureus WU1. The oropharynx of animals was swabbed in weekly intervals to enumerate the bacterial load. Each dot indicates the number of CFU per mouse. The median and standard deviation for each group of animals on a given day are indicated by the horizontal line and error bar. Bacterial colonization data sets were analyzed with two-way ANOVA and Sidak multiple comparison tests; statistically significant differences (*p<0.05; **p<0.01; ****p<0.0001) between groups of animals are indicated by asterisks.

FIG. 6. Immunization of BALB/c mice with SpA_{KKAA} promotes S. aureus JSNZ clearance from the nasopharynx. BALB/c mice were immunized with 50 μg of purified recombinant SpA_{KKAA} emulsified with CFA or PBS-mock in CFA, and boosted after 11 days with 50 μg of recombinant SpA_{KKAA} emulsified with IFA or PBS-mock in IFA. On day 0 of the colonization experiment, cohorts of BALB/c mice (n=10) mice were inoculated intra-nasally with 1×10⁸ CFU of S. aureus JSNZ. The oropharynx of animals was swabbed in weekly intervals to enumerate the bacterial load. Each dot indicates the number of CFU per mouse. The median and standard deviation for each group of animals on a given day are indicated by the horizontal line and error bar. Bacterial colonization data sets were analyzed with two-way ANOVA with Sidak multiple comparison tests; statistically significant differences (*p<0.05; **p<0.01) between the two groups of animals are indicated by asterisks.

FIGS. 7A-7C. Improved SpA vaccine. FIG. 7A: Depiction of the SpA_{KKAA}, SpA_{KKAA/A}, and SpA_{KKAA/F} variants. FIG. 7B: Binding affinity of the variants to human IgG.

FIG. 7C: Binding affinity of the variants to human IgE.

FIGS. 8A-8B. Binding assays. FIG. 8A: Western blot of the SpA variants. FIG. 8B: ELISAs of the variants to the indicated molecules.

FIGS. 9A-9B. Protein A is required for S. aureus persistent nasal colonization of mice.

FIG. 10. Protein A amino acid sequence alignment. Dark Grey: Amino acids interacting with human Fcγ fragment; Light Grey: Amino acids interacting with human Fab fragment; Asterisk denotes amino acid interacting with both human Fcγ and Fab fragments Red: Amino acids interacting with human Fcγ fragment

FIG. 11. Surface plasmon resonance (SPR) analysis demonstrating that Z domain (G29A in SpA B domain) fails to bind F(ab)2 fragment.

FIGS. 12A-12B. New SpA* variants targeting G29.

FIG. 13. New SpA* variants targeting G29.

FIG. 14. New SpA* variants targeting G29.

FIG. 15. New SpA* variants targeting G29.

FIG. 16. Depiction of a basil histamine release assay further described in Example 2.

FIGS. 17A-17B. Staphylococcal protein A (SpA). (FIG. 17A) Diagram illustrating the primary structure of the SpA precursor (with N-terminal signal peptide cleaved by signal peptidase, five immunoglobulin binding domains (IgBDs—designated E, D, A, B, C), the cell wall spanning domain designated region Xr, the LysM domain for peptidoglycan binding, and the C-terminal LPXTG sorting signal that is cleaved by sortase A), of cell wall-SpA, which is displayed on the bacterial surface, and of the released-SpA molecules that are liberated from the cell wall envelope and released into host tissues. (FIG. 17B) Secretion and sortase A-mediated cell wall anchoring of SpA and release of peptidoglycan-linked SpA by S. aureus.

FIGS. 18A-18B. Binding of SpA to the Fcγ domain of human IgG blocks the effector functions of antibodies (engagement of Fc and complement receptors) and opsonophagocytic killing of S. aureus by phagocytes. Immune evasive attributes of staphylococcal protein A. (FIG. 18A) Cell wall-anchored SpA, on the surface of S. aureus binds Fcγ of human IgG (IgG1, IgG2 and IgG4) and blocks the effector functions of antibodies to trigger opsonophagocytic killing of bacteria. (FIG. 18B) Diagram illustrating the primary structure of human IgG, its antigen-binding paratope (purple) effector (C1q, FcγRs, FcRn) and SpA binding sites.

FIGS. 19A-19B. Immune evasive attributes of staphylococcal protein A. (FIG. 19A) Immune evasive functions of SpA during S. aureus infection. Cell wall-anchored SpA, on the surface of S. aureus, binds Fcγ of human IgG and blocks the effector functions of antibodies to trigger opsonophagocytic killing of bacteria. Released-SpA crosslinks V_{H}3-idiotypic variant heavy chains of human IgG and IgM (B cell receptors) to activate B cell proliferation, class switching, somatic hypermutation and secretion of V_{H}3-idiotypic antibodies that can be crosslinked by SpA but that do not recognize S. aureus antigens, thereby blocking the development of adaptive immune responses against S. aureus and the establishment of protective immunity. (FIG. 19B) Diagram illustrating SpA-binding to and crosslinking of V_{H}3-idiotypic B cell receptors (IgM) and the activation of CD79AB signaling.

FIGS. 20A-20B. Immunoglobulin-binding domains (IgBDs) of recombinant SpA, SpA_{KKAA}, SpA_{AA}, and SpA_{KKAA}. (FIG. 20A) Diagram illustrating the primary structure of the IgBDs of recombinant SpA with an N-terminal polyhistidine tag for purification via affinity chromatography on Ni-NTA from the cytoplasm of E. coli. The amino acid sequence of the IgBD-E domain is displayed below. Positions of three α-helices for each IgBD (H1, H2, and H3) are indicated. SpA_{KK} and SpA_{KKAA} harbor amino acid substitutions at Q^{9,10}K (Gln^{9,10}Lys). SpA_{KK} and SpA_{KKAA} harbor amino acid substitutions at D^{36,37}A (Asp^{36,37}Lys). Numbering refers to the position of amino acids in the B-IgBD. (FIG. 20B) Amino acid sequence alignment of the five IgBDs of SpA. Conserved amino acids are indicated by a period (.). Gaps in alignment are indicated by a dash (-). Non-conserved amino acids are listed in the single letter code. As reported by Graille et al. (138), SpA residues involved in IgG Fcγ binding are highlighted in red. SpA residues responsible for V_{H}3-heavy chain binding are highlighted in green. The pink residue (Q³²) contributes both to Fcγ and V_{H}3 binding.

FIGS. 21A-21B. SpA associated V_{H}3-crosslinking activity and anaphylaxis. (FIG. 21A) Diagram illustrating the structure of human activating Fcγ and Fcε receptors as well as their V_{H}3-idiotypic IgG and IgE ligands. (FIG. 21B) SpA crosslinking of V_{H}3-idiotypic IgG or IgE that engage FcγR and FcεR receptors, respectively, on basophils or mast cells triggers the release of histamine, of inflammatory mediators and of cytokines that promote anaphylactic reactions, vasodilation and shock. Although not depicted in (FIG. 21B), both mast cells and basophils express FcγR and FcεR receptors and respond to SpA-crosslinking of V_{H}3-idiotypic IgG bound to FcγR or to SpA-crosslinking of V_{H}3-idiotypic IgE bound to FcεR receptors with the release of histamine, pro-inflammatory mediators and cytokines.

FIG. 22. Anaphylactic activity of SpA vaccine candidates in mice. μMT mice (n=5) were sensitized with VH3 IgG by intradermal injection in the ear. Candidate vaccine antigens or PBS control were injected intravenously 24 hours later followed by Evans blue injection. Extravasation of the dye was quantified following extraction from ear tissues after 30 minutes, by spectrophotometric measurement at 620 nm. Data were obtained from three independent experiments. One-way ANOVA with Bonferroni's Multiple Comparison Test was performed for statistical analysis of the data. Symbols: ns, not significant; *, P<0.05; **, P<0.01; ***, P<0.001; ****, P<0.0001.

FIGS. 23A-23B. Degranulation of mast cells. Cultured human mast cells (LAD2) were sensitized overnight with VH3 IgE, washed, and either left untreated (PBS) or exposed for 1 hour to SpA as a positive control or test articles SpA_{KKAA}, SpA_{Q9,10K/S33E}, SpA_{Q9,10K/S33T}, or SpA-KR. β-Hexosaminidase and histamine levels were measured in cell pellets, as well as in supernatants. The percentage of β-hexosaminidase (FIG. 23A) and amount of histamine (FIG. 23B) release are shown. One-way ANOVA with Bonferroni's Multiple Comparison Test was performed for statistical analysis of the data. Symbols: ns, not significant; *, P<0.05; **, P<0.01; ***, P<0.001; ****, P<0.0001.

FIGS. 24A-24E. Immunization with SpA_{KKAA} or SpA_{Q9,10K/S33E} or SpA_{Q9,10/S33T} promotes progressive decolonization. Cohorts of C57BL/6 mice (n=10) were inoculated intranasally with 1×10⁸ CFU of S. aureus WU1. (FIG. 24A, 24B, 24D) Mice were swabbed in the throat weekly to enumerate the bacterial load. (FIG. 24C, 24E) Stool samples were collected weekly following inoculation to enumerate the bacterial load. In panel (FIG. 24A), animals were immunized with adjuvant-PBS or -SpA_{KKAA}. In panels (FIG. 24B-24C), animals were immunized with adjuvant-SpA_{KKAA} or -SpA_{Q9,10K/S33E}; the same cohorts of animals were monitored for bacterial loads in the throat (FIG. 24B) and stool samples (FIG. 24C). In panels (FIG. 24D-24E), animals were immunized with adjuvant-PBS or -SpA_{KKAA}. or -SpA_{Q9,10K/S33E} or -SpA_{Q9,10K/S33T}; the same cohorts of animals were monitored for bacterial loads in the throat (FIG. 24D) and stool samples (FIG. 24E). Each square indicates the number of CFU per milliliter per throat swab or per gram of stool. The median and standard deviation for each group of animals on a given day are indicated by the horizontal lines and error bars. The data was examined with the two-way analysis of variance with Sidak multiple-comparison tests (*, P<0.05). In panels (FIG. 24D-24E), each group of data (each of 1-8) represents data from mock, SpA_{KKAA}, SpA_{Q9,10K/S33E}, or SpA_{Q9,10K/S33T}, respectively. No statistical differences were noted between the two groups in panels 24B and 24C.

FIG. 25A-25C. Protective activity of SpA vaccine candidates in the mouse model of bloodstream infection. Three-week-old BALB/c mice (n=15) were immunized with SpA_{KKAA} or SpA_{Q9,10K/S33E} or SpA_{Q9,10K/S33T} or PBS control. Mock or booster immunizations occurred on day 11. On day 20, mice were bled to evaluate serum half-maximal antibody titers to vaccine candidates, denoted as SpA* on they axis. Each group of three bars represents, from left to right, SpA_{KKAA}, SpA_{Q9,10K/S33E}, and Spa_{AQ9,10K/S33T}. (FIG. 25A). On day 21, mice were challenged with 5×10⁶ CFU of S. aureus USA300 (LAC) into the periorbital venous sinus of the right eye. Fifteen days post-challenge, animals were euthanized to enumerate staphylococcal loads in kidneys (FIG. 24B) and to enumerate abscess lesions (FIG. 24C). One-way ANOVA with Bonferroni's Multiple Comparison Test was performed for statistical analysis of the data. Symbols: ns, not significant; *, P<0.05; **, P<0.01; ***, P<0.001; ****, P<0.0001.

FIG. 26A-26C. Interaction between SpA vaccine candidates and SpA-neutralizing monoclonal antibody 3F6. 3F6 antibodies, recombinant rMAb 3F6 from HEK293 F cells (FIG. 26A, rMAb 3F6) or mouse hybridoma monoclonal antibody (FIG. 26B, hMAb 3F6) were serially diluted across enzyme-linked immunosorbent assay plates coated with either SpA_{KKAA} or SpA_{Q9,10K/S33E} or SpA_{Q9,10K/S33T} or PBS control. (FIG. 26C) Association constants calculated using GraphPad Prism software.

FIG. 27. Immunization, challenge and sampling schedule of minipigs. Male Gottingen Minipigs (3 pigs per group) were immunized intramuscularly on 3 separate occasions at 3-week intervals. Following vaccination, the pigs were challenged with a clinically-relevant S. aureus strain (CC398 or CC8 USA300). Blood samples were taken prior to each vaccination and at regular intervals during the infection period. Blood and serum analysis were performed to evaluate serum immunoglobulin quantity and function. At day 8 post-infection, pigs were euthanized and the bacterial burden at the surgical site and internal organs was determined. FIG. 27 shows an overview of the in vivo experimental design. Pigs are bled and immunized at days −63, −42, and −21; bled and infected at day 0, bled at days +1, +2, and +3, and euthanized and necropsied at day +8.

FIGS. 28A-28D. Immunization with LukAB and SpA* resulted in generation of specific LukAB and SpA antibodies in minipigs. Minipigs were immunized on 3 separate occasions with 100 μg LukAB toxoid, 50 μg SpA*, or a mixture of 100 μg LukAB toxoid+50 μg SpA*. Antigens in each group were administered with 25 μg of MPL, and 25 μg of QS-21 adjuvant per animal and vaccination. A control group was vaccinated with 25 μg of MPL, and 25 μg of QS-21 adjuvant only. Serum samples were evaluated for IgG against wildtype LukAB (FIGS. 28A and 28C) and against SpA* (FIGS. 28B and 28D). Each dot represents the EC₅₀ titer of an individual animal on days −63 (pre-immun sera), day −42 (three weeks after first immunization), day −21 (three weeks after second immunization), day 0 (three weeks after third immunization, prior to challenge) and +8 (at necropsy). The bars show geometric mean EC₅₀ titers for each group. FIG. 28A: Anti-LukAB antibody responses measured in study 1 (challenge strain CC398); FIG. 28B: Anti-SpA* antibody responses measured in study 1 (challenge strain CC398); FIG. 28C: Anti-LukAB antibody responses measured in study 2 (challenge strain USA300); FIG. 28D: Anti-SpA* antibody responses measured in study 2 (challenge strain USA300).

FIGS. 29A-29B. Immunization with LukAB and SpA* resulted in generation of antibodies that neutralize the activity of the LukAB toxin. Minipigs were immunized on 3 separate occasions with 100 μg LukAB toxoid, 50 μg SpA*, or a mixture of 100 μg LukAB toxoid+50 μg SpA*. Antigens in each group were administered with 25 μg of MPL, and 25 μg of QS-21 adjuvant per animal and vaccination. A control group was vaccinated with 25 μg of MPL, and 25 μg of QS-21 adjuvant only. Serum samples were evaluated for neutralization of the LukAB toxin. Each dot represents the IC₅₀ titer of an individual animal on days −63 (pre-immune sera), −21 (three weeks after second immunization), day 0 (three weeks after third immunization, prior to challenge) and +8 (at necropsy). Bars indicate the geometric mean titers for each group. FIG. 29A: LukAB toxin neutralization in study 1 (challenge strain CC398); FIG. 29B: LukAB toxin neutralization in study 2 (challenge strain USA300).

FIGS. 30A-30D. Immunization with LukAB and SpA* resulted in decrease of the colony forming units (cfu) at the surgical site and spleen of minipigs. Minipigs were immunized on 3 separate occasions with 100 μg LukAB toxoid, 50 μg SpA*, or a mixture of 100 μg LukAB toxoid+50 μg SpA*. Antigens in each group were administered with 25 μg of MPL, and 25 μg of QS-21 adjuvant per animal and vaccination. A control group was vaccinated with 25 μg of MPL, and 25 μg of QS-21 adjuvant only. Three weeks after the third immunization animals were challenged with S. aureus CC398 strain (study 1) or a CC8 USA300 strain (study 2) in a surgical site infection model. At day 8 post-infection, animals were euthanized and the surgical site and organs were necropsied. The bacterial burden was determined by spiral plating followed by cfu counting. Each dot represents the log₁₀ value of cfu in the muscle and spleen of an individual animal. The line indicates the geometric mean of each group. The dotted line indicates the limit of detection. FIG. 30A: cfu in total muscle, study 1 (challenge strain CC398); FIG. 30B: cfu in spleen, study 1 (challenge strain CC398); FIG. 30C: cfu in total muscle, study 2 (challenge strain USA300); FIG. 30D: cfu in spleen, study 2 (challenge strain USA300).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., Staphylococcus LukA, LukB, SpA polypeptides and the polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector,” refers to e.g. any number of nucleic acids into which a desired sequence can be inserted, e.g., be restriction and ligation, for transport between genetic environments or for expression in a host cell. Nucleic acid vectors can be DNA or RNA. Vectors include, but are not limited to, plasmids, phage, phagemids, bacterial genomes, viruse genomes, self-amplifying RNA, replicons.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel. Moreover, an “isolated nucleic acid fragment” or “isolated peptide” is a nucleic acid or protein fragment that is not naturally occurring as a fragment and/or is not typically in the functional state.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the disclosure in a recipient subject. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response can also involve activation of monocytes, macrophages, NK cells, basophils, dendritic cells, astrocytes, microglia cells, eosinophils, or other components of innate immunity. As used herein “active immunity” refers to any immunity conferred upon a subject by administration of an antigen.

LukA, LukB, and/or SpA Polypeptides and Polynucleotides Encoding the Same

It was found herein that following vaccination with the vaccine combinations of the present invention, vaccine antibodies (i.e., that were elicited following vaccination) generated against both SpA variant polypeptides and mutant LukAB polypeptides provided synergistic protection and efficient S. aureus killing due to a dual-mechanism. On the one hand, the neutralization of the SpA molecule prevented the upside-down binding of antibodies (IgG Fc binding) and prevented B-cell dysregulation by disrupting SpA binding to V_{H}3. On the other hand, the neutralization of the LukAB toxins prevented the lysing of phagocytic cells by LukAB, and, therefore, allowed for human neutrophils to remain functional and capable of eliminating S. aureus by opsonophagocytosis. The antibody response was productive, as the antibodies bound the respective target, and the phagocytic cells were capable of killing, i.e., there was a clear and additive synergistic effect of neutralizing both SpA and LukAB.

In a general aspect, the invention relates to immunogenic compositions comprising a S. aureus protein A (SpA) variant and a mutant staphylococcal leukocidin subunit polypeptide comprising (i) a LukA polypeptide, (ii) a LukB polypeptide, and/or (iii) a LukAB dimer polypeptide, wherein the LukA polypeptide, LukB polypeptide, and/or LukAB dimer polypeptide have one or more amino acid substitutions, deletions, or a combination thereof in the LukA polypeptide, the LukB polypeptide, or the LukAB dimer polypeptide. The one or more amino acid substitutions, deletions, or a combination thereof disrupts the ability of the LukA, LukB, and/or LukAB polypeptides to form pores in the surface of eukaryotic cells, thereby reducing the toxicity of the LukA and/or LukB polypeptide or the mutant LukAB dimer polypeptide relative to the corresponding wild-type LukA and/or LukB polypeptide or LukAB dimer polypeptide. The Staphylococcus protein A (SpA) variant polypeptide comprises one or more amino acid substitutions, deletions, insertions, or combinations thereof, such that the SpA variant polypeptide has disrupted the ability to bind IgG Fc and/or V_{H}3 resulting in a SpA variant polypeptide with reduced toxicity as compared to a wild-type SpA polypeptide or other SpA variant polypeptide, such as the SpA_{KKAA} polypeptide.

Staphylococcal Leukocidin Subunit Polypeptides: LukA Polypeptides, LukB Polypeptides, and/or LukAB Dimer Polypeptides

In a general aspect, the invention relates to immunogenic compositions comprising a mutant staphylococcal leukocidin subunit polypeptide or polynucleotides (DNA or RNA) encoding the same. The mutant staphylococcal leukocidin subunit polypeptide can comprise (i) a LukA polypeptide, (ii) a LukB polypeptide; and/or (iii) a LukAB dimer polypeptide. The LukA polypeptide, the LukB polypeptide, and/or the LukAB dimer polypeptide can comprise one or more amino acid substitutions, deletions, insertions, or a combination thereof in the LukA polypeptide, LukB polypeptide, and/or LukAB dimer polypeptide. In certain embodiments, the one or more amino acid substitutions, deletions, insertions, or a combination thereof are in the LukAB protomer/protomer interface region, the LukAB dimer/dimer interface region, the LukB membrane-binding cleft region, the LukB pore forming region, or any combination thereof such that the ability of the leukocidin subunits to form dimers, to oligomerize, to form pores on the surface of eukaryotic cells, or any combination thereof is disrupted. Disruption can cause a reduction in toxicity of the mutant staphylococcal leukocidin subunit polypeptide.

In certain embodiments, the one or more amino acid substitutions, deletions, insertions, or combinations thereof do not significantly reduce the immunogenicity of the mutant leukocidin subunit polypeptide relative to the corresponding wild-type leukocidin subunit polypeptide. In certain embodiments, the mutant staphylococcal subunit polypeptide is immunogenic and elicits an immune response that can comprise antibodies that can neutralize the action of the wild-type staphylococcal leukocidin subunit polypeptide. In certain embodiments, the mutant staphylococcal leukocidin subunit polypeptide or polynucleotides (DNA or RNA) encoding the same can be immunogenic and elicits antibodies that can more effectively neutralize the action of the wild-type staphylococcal subunit polypeptide relative to the corresponding wild-type leukocidin subunit polypeptide.

The terms “staphylococcal leukocidin subunit polypeptide,” “staphylococcal leukocidin subunit,” “LukA subunit,” “LukA polypeptide,” “LukB subunit,” “LukB polypeptide,” “LukAB dimer polypeptide,” and the like, as used herein, encompass mature or full length staphylococcal leukocidin subunits (e.g., LukA and/or LukB), and fragments, variants or derivatives of mature or full length staphylococcal leukocidin subunits (e.g., LukA and/or LukB), and chimeric and fusion polypeptides comprising mature or full length staphylococcal leukocidin subunits (e.g., LukA and/or LukB) or one or more fragments of mature or full length staphylococcal leukocidin subunits (e.g., LukA and/or LukB). In certain embodiments, mutant staphylococcal leukocidin subunit polypeptides, as disclosed herein, are reduced in toxicity relative to a corresponding wild-type staphylococcal leukocidin subunit polypeptide and/or are not significantly reduced in immunogenicity relative to a corresponding wild-type staphylococcal leukocidin subunit polypeptide.

Pore forming toxins, e.g., single-component alpha-hemolysin and the bi-component hemolysins and leukotoxins, play an important role in staphylococcal immune evasion. These toxins can kill immune cells and cause tissue destruction, thereby weakening the host during the first stage of infection and promoting bacterial dissemination and metastatic growth. The bi-component toxin LukAB, comprising LukA and LukB subunits, is unique in that it is secreted as a dimer, which then octamerizes on the surface of cells to form pores. In contrast, e.g., the two PVL components, LukS-PV and LukF-PV, are secreted separately and form the pore-forming octameric complex upon binding of LukS-PV to its receptor and subsequent binding of LukF-PV to LukS-PV (Miles et al., Protein Sci. 11(4):894-902 (2002); Pedelacq et al., Int. J. Med. Microbiol. 290(4-5):395-401 (2000)). Targets of PVL can include, e.g., polymorphonuclear neutrophils (PMNs), monocytes, and macrophages.

Other bi-component toxins have been characterized: S components HlgA and HlgC and the F component HlgB for γ-hemolysin; LukS-PV, LukF-PV, LukE (S) and LukD (F); and LukM (S) and LukF-PV-like (F) (WO2011/112570). Due to their close similarity, these S components can combine with an F component and form an active toxin with different target specificity (Ferreras et al., Biochim Biophys Acta 1414(1-2):108-26 (1998); Prevost et al., Infect. Immun. 63(10):4121-9 (1995)). γ-hemolysin is strongly hemolytic and 90% less leukotoxic than PVL, while PVL is non-hemolytic. However, HlgA or HlgC paired with LukF-PV promotes leukotoxic activity (Prevost et al., Infect. Immun. 63(10):4121-9 (1995)). PVL and other leukotoxins lyse neutrophils, and Hlg is hemolytic (Kaneko et al., Biosci. Biotechnol. Biochem. 68(5):981-1003 (2004)) and was also reported to lyse neutrophils (Malachowa et al., PLoS One 6(4):e18617 (2011)). While PVL subunits are phage derived, Hlg proteins are derived from Hlg locus and found in 99% of clinical isolates (Kaneko et al., supra). Hlg subunits are upregulated during S. aureus growth in blood (Malachowa et al., supra), and Hlg was shown to be involved in the survival of S. aureus in blood (Malachowa et al., Virulence 2(6) (2011)). The mutant USA 300 Δ-hlgABC has reduced capacity to cause mortality in a mouse bacteremia model (Malachowa et al., PLoS One 6(4):e18617 (2011)). LukED toxin is critical for bloodstream infections in mice (Alonzo et al., Mol. Microbiol. 83(2):423-35 (2012)). LukAB has been described to synergize with PVL to enhance PMN lysis (Ventura et al., PLoS One 5(7):e11634 (2010); LukAB referred to as LukGH therein).

The sequence similarity among the five different leukotoxins leukocidines γ-hemolysin (HlgAB and HlgCB), leucocidin E/D (LukED), Panton-Valine leucocidin (PVL), and leukocidin A/B (LukAB, also known as LukGH) ranges from 60% to 80%, with the exception of LukAB, which is only 30-40% similar to the others. Although all leukocidins can target and kill polymorphonuclear cells, they differ in their potency to do so. LukAB is extremely effective at killing human PMNs, including neutrophils. LukAB differs from the other leukotoxins as it is secreted as a heterodimer that specifically binds to the I domain of human CD11b subunit of integrin αM/β2 receptor, which is responsible for the specific binding and killing of human PMNs by LukAB (http://www.pnas.org/content/110/26/10794.full.pdf). Neutralization of these toxins, and in particular that of LukAB, by vaccine-induced antibodies is believed to strongly reduce the killing of neutrophils during an S. aureus infection, thereby preserving the ability of the host immune system to clear the pathogen.

LukED

Although LukED is not a component of the core S. aureus genome, it is conserved within predominant lineages associated with invasive infections. Unlike LukAB and PVL, which display activity in a species-specific manner, LukED displays broad activity across species, including comparable toxicity against murine, rabbit, and human leukocytes. LukED displays lytic activity against cells expressing the receptors CCR5, including macrophages, T cells, and dendritic cells, and CXCR1, CXCR2, including primary neutrophils, monocytes, natural killer cells, and a subset of CD8+ T cells (Spaan et al., 2017 Nat Rev Microbiol 15: 435-47). These activities contribute to the evasion of both the innate and adaptive arms of the immune system to facilitate disease progression. In animal models of infection, LukED elicits a proinflammatory response and contributes to replication in the liver and kidney through the killing of infiltrating neutrophils. LukED also binds erythrocytes in a DARC (Duffy antigen receptor for chemokines)-dependent manner, resulting in hemolysis, release of hemoglobin and the promotion of S. aureus growth through the acquisition of iron (Spaan et al., 2015 Cell Host Microbe 18: 363-70).

Hla

Alpha hemolysin (alpha toxin, Hla) contributes to pathogenesis and lethal infection through multiple activities, including direct toxicity and lysis of erythrocytes and other cells and immunomodulation. Hla is secreted as a soluble monomeric protein that binds to the ADAM10 receptor and assembles into a heptameric-barrel pore complex that is structurally very similar to those of the bi-component β-PFT, such as LukAB and LukED. In addition to erythrocytes, at high concentrations Hla can lyse numerous other cell types expressing ADAM10, including macrophages and monocytes. Cell lysis mediated by Hla is dependent upon both toxin concentration and the level of ADAM10 expression. The role of Hla in S. aureus virulence is well established in numerous animal models, including sepsis, pneumonia, skin infections and others (Berube and Bubeck Wardenburg, 2013 Toxins 5: 1140-66). Hla is expressed during human infection and is immunogenic, with higher titers of anti-Hla antibodies associated with a reduced risk of S. aureus sepsis (Adhikari et al., 2012 J Infect Dis 206: 915-23). Additionally, S. aureus isolates displaying elevated levels of Hla expression are associated with invasive disease. Due to it's role in S. aureus virulence, Hla has been extensively explored as a vaccine antigen. An attenuated mutant, Hla_{H35L}, which cannot form active pore complexes, demonstrated protective efficacy in several mouse infection models (Bubeck Wardenburg and Schneewind, 2008 J Exp Med 205: 287-94). Hla antigens derived from the N-terminal 62 residues (Adhikari et al., 2016 Vaccine 34: 6402-7) or from a deletion of the stem domain (Fiaschi et al., 2016 Vaccine 23: 442-450) were also immunogenic and elicited protective immune responses.

“LukA polypeptides,” as described herein, are polypeptides native to staphylococcal organisms (e.g., Staphylococcus aureus), which specifically target and bind human phagocytes (but not endothelial cells, or murine cells). Once the LukA polypeptide is bound to a phagocyte membrane, LukA oligomerizes with a staphylococcal F-subunit leukocidin (e.g., LukF-PVL, LukD and HlgB, and LukB, as disclosed herein). Upon oligomerization, the polypeptides form a transmembrane pore (collectively referred to as LukA activity).

LukA polypeptides typically comprise 351 amino acid residues. The amino-terminal 27 amino acid residues represent the native secretion/signal sequence, and, thus, the mature, secreted form of LukA, is represented by amino acid residues 28-351, and can be referred to as “LukA₍₂₈₋₃₅₁₎” or “mature LukA.” Correspondingly, the immature form of LukA can be referred to herein as “LukA₍₁₋₃₅₁₎.” Examples of immature LukA polypeptides isolated from different strains of Staphylococcus aureus include the LukA polypeptides of SEQ ID NOs:2-14. SEQ ID NO:1 provides a consensus LukA polypeptide sequence based on the alignment of SEQ ID NOs:2-14, as disclosed in WO2011/140337, which is incorporated by reference herein in its entirety. Examples of mature LukA polypeptides corresponding to the immature LukA polypeptides of SEQ ID NOs:1-14 with the secretion/signal sequence deleted include SEQ ID NOs:15-28, respectively.

“LukB polypeptides,” as described herein, are polypeptides native to staphylococcal organisms (e.g., Staphylococcus aureus), which exhibit the activity profile of a F-subunit leukocidin (e.g., LukF-PVL, LukD and HlgB). LukB polypeptides specifically oligomerize with a staphylococcal S-subunit leukocidin (e.g., LukS-PVL, LukE and HlgC, and LukA, as disclosed herein), which is bound to a human phagocyte. Upon oligomerization it will form a transmembrane pore in the phagocyte (collectively referred to as LukB activity).

LukB polypeptides typically comprise 339 amino acid residues. The amino terminal (N-terminal) 29 amino acid residues represent the secretion/signal sequence, and, thus, the mature, secreted form of LukB is represented by amino acid residues 30-339, and can be referred to as “LukB₍₃₀₋₃₃₉₎” or “mature LukB.” Correspondingly, the immature form of LukB can be referred to herein as “LukB₍₁₋₃₃₉₎.” Examples of immature LukB polypeptides isolated from different strains of Staphylococcus aureus include the LukB polypeptides of SEQ ID NOs:30-41. SEQ ID NO:29 provides a consensus LukB polypeptide sequence based on the alignment of SEQ ID NOs:30-41, as disclosed in WO2011/140337, which is incorporated by reference herein in its entirety. Examples of mature LukB polypeptides corresponding to the immature LukB polypeptides of SEQ ID NOs:29, 30, and 32-41 with the secretion/signal sequence deleted include SEQ ID NOs:42-53, respectively.

Reference is made to LukA polypeptides, LukB polypeptides, and LukAB dimer polypeptides herein. One of ordinary skill in the art would understand that LukA can also be referred to as LukH and that LukB can also be referred to as LukG, see, e.g., U.S. Pat. No. 8,431,687 (LukAB); Badarau et al., JBC 290(1):142-56 (2015) (LukGH); and Badarau et al., MABS 9(7):1347-60 (2016) (LukGH).

LukA and LukB polypeptides can comprise one or more additional amino acid insertions, substitutions, and/or deletions, e.g., one or more amino acid residues within SEQ ID NOs:1-28 and/or SEQ ID NOs:29-53 can be substituted with another amino acid of similar polarity, which can act as a functional equivalent, resulting in a silent alteration. A change of an amino acid with one of similar polarity could result in a LukA and/or LukB polypeptide with the same basic properties as a wild type LukA and/or LukB polypeptides.

In certain embodiments, non-conservative alterations can be made to a LukA and/or LukB polypeptide for the purpose of inactivating or detoxifying LukA and/or LukB. In certain embodiments, LukA polypeptides can comprise a deletion at amino acid residue positions 342-351 of SEQ ID NOs:1-14 (except for SEQ ID NOs:4-6, which contain 9 amino acids in these positions, and, thus, can comprise a deletion at amino acid residue positions 342-350). The deletion at amino acid residue positions 342-351 would occur at amino acid residue positions 315-324 of SEQ ID NOs:15-28 (except for SEQ ID NOs:18-20, which contain 9 amino acids in these positions, and, thus, can comprise a deletion at amino acid residue positions 315-323). The detoxified LukA and/or LukB can be used in the immunogenic compositions disclosed herein.

In certain embodiments, provided herein are LukA polypeptides comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1-28.

In certain embodiments, the one or more mutations comprise 1, 2, 3, 4, 5, 6, 7, 8, 9, 10, 11, 12, 13, 14, or 15 amino acid substitutions, deletions, insertions, or a combination thereof in the LukA polypeptide. In certain embodiments, the one or more substitutions, deletions, insertions, or a combination thereof are at conserved residues in the LukAB protomer/protomer interface region, the dimer/dimer interface region, or any combination thereof. The ability of the mutant LukA polypeptide to form dimers, to oligomerize, to form pores on the surface of the eukaryotic cells, or any combination thereof can, for example, be disrupted. The toxicity of the mutant LukA polypeptide relative to the corresponding wild-type LukA polypeptide can, for example, be reduced. The immunogenicity of the mutant LukA polypeptide and/or LukAB dimer polypeptide relative the corresponding wild-type LukA polypeptide and/or LukAB dimer polypeptide can, for example, not be significantly reduced. LukA polypeptides comprising one or more mutations are described in WO2018/232014, which is incorporated by reference herein in its entirety.

In certain embodiments, a substitution of the glutamic acid residue at position 323 of the mature LukA polypeptide can be made for the purpose of inactivating or detoxifying the LukAB dimer. In certain embodiments, the substitution of the glutamic acid residue at position 323 of the mature LukA polypeptide with an alanine residue, i.e., an E323A substitution, can be made for the purpose of inactivating or detoxifying the LukAB dimer polypeptide (DuMont et al., Infect. Immun. (2014)).

In certain embodiments, provided herein are LukB polypeptides comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:29-53.

In certain embodiments, the one or more mutations comprise 1, 2, 3, 4, 5, 6, 7, 8, 9, 10, 11, 12, 13, 14, or 15 amino acid substitutions, deletions, insertions, or a combination thereof in the LukB polypeptide. In certain embodiments, the one or more substitutions, deletions, insertions, or a combination thereof are at conserved residues in the LukAB protomer/protomer interface region, the dimer/dimer interface region, the LukB membrane-binding cleft region, the LukB pore forming region, or any combination thereof. The ability of the mutant LukB polypeptide to form dimers, to oligomerize, to form pores on the surface of the eukaryotic cells, or any combination thereof can, for example, be disrupted. The toxicity of the mutant LukB polypeptide relative to the corresponding wild-type LukB polypeptide can, for example, be reduced. The immunogenicity of the mutant LukB polypeptide and/or LukAB dimer polypeptide relative the corresponding wild-type LukB polypeptide and/or LukAB dimer polypeptide can, for example, not be significantly reduced. LukB polypeptides comprising one or more mutations are described in WO2018/232014, which is incorporated by reference herein in its entirety.

In certain embodiments, provided herein are mutant LukAB dimer polypeptides comprising an amino acid sequence with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1-28 and an amino acid sequence with at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:29-53.

In certain embodiments, a mutant staphylococcal leukocidin subunit polypeptide comprises a mutation in the LukAB protomer/protomer interface region. The mutation can, for example, result in the formation of an incomplete, larger leukocidin octamer ring; reduce or abolish hemolytic/leukotoxic activity of the toxin; or any combination thereof. In certain embodiments, the mutation can comprise a substitution in a LukA polypeptide corresponding to amino acid R49 of SEQ ID NO:15; a substitution in a LukA polypeptide corresponding to amino acid L61 of SEQ ID NO:15; a substitution in a LukB polypeptide corresponding to amino acid D49 of SEQ ID NO:42; or a combination thereof. In certain embodiments, the substitution in the LukA polypeptide corresponding to amino acid R49 of SEQ ID NO:15 is glutamate (E). The substitution in the LukA polypeptide can disrupt the salt bridge between LukA R49 of SEQ ID NO:15 and LukB D49 of SEQ ID NO:42. In certain embodiments, the substitution in the LukA polypeptide corresponding to amino acid L61 of SEQ ID NO:15 is an asparagine (N), glutamine (Q), or arginine (R) substitution. The substitution in the LukA polypeptide can disrupt the hydrophobic pocket found within the LukAB protomer/protomer interface. In certain embodiments, the substitution in the LukB polypeptide corresponding to amino acid D49 of SEQ ID NO:42 is an alanine (A) or a lysine (K) substitution. The substitution in the LukB polypeptide can disrupt the salt bridge between LukB D49 of SEQ ID NO:42 and LukA R49 of SEQ ID NO:15.

In certain embodiments, a mutant staphylococcal leukocidin subunit polypeptide comprises a mutation in the LukAB dimer/dimer interface region. The mutation can, for example, disrupt the LukAB dimer formation, can disrupt LukAB oligomerization on the surface of the eukaryotic cell, or a combination thereof. In certain embodiments, the mutation can comprise a substitution in a LukA polypeptide corresponding to amino acid D39 of SEQ ID NO:15; a substitution in a LukA polypeptide corresponding to amino acid D75 of SEQ ID NO:15; a substitution in a LukA polypeptide corresponding to amino acid K138 of SEQ ID NO:15; a substitution in a LukA polypeptide corresponding to amino acid D197 of SEQ ID NO:15; a substitution in a LukB polypeptide corresponding to amino acid K12 of SEQ ID NO:42; a substitution in a LukB polypeptide corresponding to amino acid K19 of SEQ ID NO:42; a substitution in a LukB polypeptide corresponding to amino acid R23 of SEQ ID NO:42; a substitution in a LukB polypeptide corresponding to amino acid K58 of SEQ ID NO:42; a substitution in a LukB polypeptide corresponding to amino acid E112 of SEQ ID NO:42; a substitution in a LukB polypeptide corresponding to amino acid K218 of SEQ ID NO:42; or any combination thereof.

In certain embodiments, the substitution in the LukA polypeptide corresponding to amino acid D39 of SEQ ID NO: 15 is an alanine (A) or arginine (R) substitution. The substitution at D39 of SEQ ID NO: 15 can disrupt the salt bridge between LukA D39 of SEQ ID NO:15 and LukB K58 of SEQ ID NO:42.

In certain embodiments, the substitution in the LukA polypeptide corresponding to amino acid D75 of SEQ ID NO: 15 is an alanine (A) substitution. The substitution at D75 of SEQ ID NO:15 can disrupt the salt bridge between LukA D75 of SEQ ID NO:15 and LukB R23 of SEQ ID NO:42.

In certain embodiments, the substitution in the LukA polypeptide corresponding to amino acid K138 of SEQ ID NO:15 is an alanine (A) substitution. The substitution at K138 of SEQ ID NO:15 can disrupt the salt bridge between LukA K138 of SEQ ID NO:15 and LukB E112 of SEQ ID NO:42.

In certain embodiments, the substitution in the LukA polypeptide corresponding to amino acid D197 of SEQ ID NO:15 is an alanine (A) or lysine (K) substitution. The substitution at D197 of SEQ ID NO:15 can disrupt the salt bridge between LukA D197 of SEQ ID NO:15 and LukB K218 of SEQ ID NO:42.

In certain embodiments, the substitution in the LukB polypeptide corresponding to K12 of SEQ ID NO:42 is an alanine (A) substitution. In certain embodiments, the substitution in the LukB polypeptide corresponding to K19 of SEQ ID NO:42 is an alanine (A) substitution. In certain embodiments, the substitution in the LukB polypeptide corresponding to R23 of SEQ ID NO:42 is an alanine (A) or glutamate (E) substitution. In certain embodiments, the LukB polypeptide can comprise a triple mutation corresponding to K12, K19, and R23 of SEQ ID NO:42. The substitution at K12, K19, and/or R23 of SEQ ID NO:42 can disrupt at least the salt bridge between LukB R23 of SEQ ID NO:42 and LukA D75 of SEQ ID NO:15.

In certain embodiments, the substitution in the LukB polypeptide corresponding to K58 of SEQ ID NO:42 is an alanine (A) or glutamate (E) substitution. The substitution at K58 of SEQ ID NO:42 can disrupt the salt bridge between LukB K58 of SEQ ID NO:42 and LukA D39 of SEQ ID NO: 15.

In certain embodiments, the substitution in the LukB polypeptide corresponding to E112 of SEQ ID NO:42 is an alanine (A) substitution. The substitution at E112 of SEQ ID NO:42 can disrupt the salt bridge between LukB E112 of SEQ ID NO:42 and LukA K138 of SEQ ID NO:15.

In certain embodiments, the substitution in the LukB polypeptide corresponding to K218 of SEQ ID NO:42 is an alanine (A) substitution. The substitution at K218 of SEQ ID NO:42 can disrupt the salt bridge between LukB K218 of SEQ ID NO:42 and LukA D197 of SEQ ID NO:15.

In certain embodiments, a mutant staphylococcal leukocidin subunit polypeptide comprises a mutation in the LukB membrane-binding cleft region. The mutation can, for example, disrupt the interaction of the LukB subunit with the polar head groups of the lipid bilayer of a eukaryotic cell. In certain embodiments, the mutation can comprise a substitution in a LukB polypeptide corresponding to amino acid H180 of SEQ ID NO:42; a substitution in a LukB polypeptide corresponding to amino acid E197 of SEQ ID NO: 42; a substitution in a LukB polypeptide corresponding to R203 of SEQ ID NO:42; or any combination thereof. In certain embodiments, the substitution in the LukB polypeptide corresponding to H180 of SEQ ID NO:42 is an alanine (A) substitution; the substitution in the LukB polypeptide corresponding to E197 of SEQ ID NO:42 is an alanine (A) substitution; and the substitution in the LukB polypeptide corresponding to R203 of SEQ ID NO:42 is an alanine (A) substitution.

In certain embodiments, a mutant staphylococcal leukocidin subunit polypeptide comprises a mutation in the LukB pore forming region. The mutation can, for example, obstruct the cytoplasmic edge of the LukAB pore formed on the surface of a eukaryotic cell, thereby obstructing pore formation. In certain embodiments, the mutation in the pore forming region comprises a deletion of amino acids F125 to T133 of SEQ ID NO:42; and in certain aspects further comprises the insertion of one, two, three, four, or five glycine (G) residues after the amino acid corresponding to D124 of SEQ ID NO:42.

In certain embodiments, the LukAB dimer polypeptide comprises (a) a LukA polypeptide with an L61R amino acid substitution corresponding to SEQ ID NO:15 and a LukB polypeptide with a D49K amino acid substitution corresponding to SEQ ID NO:42; (b) a LukA polypeptide with an L61R amino acid substitution corresponding to SEQ ID NO: 15 and a LukB polypeptide with an R23A amino acid substitution corresponding to SEQ ID NO:42; (c) a LukA polypeptide with an L61R amino acid substitution corresponding to SEQ ID NO: 15 and a LukB polypeptide with an R23E amino acid substitution corresponding to SEQ ID NO:42; (d) a LukA polypeptide with an L61R amino acid substitution corresponding to SEQ ID NO: 15 and a LukB polypeptide with an E112A amino acid substitution corresponding to SEQ ID NO:42; (e) a LukA polypeptide with an L61R amino acid substitution corresponding to SEQ ID NO: 15 and a LukB polypeptide with an R203A amino acid substitution corresponding to SEQ ID NO:42; (f) a LukA polypeptide with an L61R amino acid substitution corresponding to SEQ ID NO: 15 and a LukB polypeptide with a K218A amino acid substitution corresponding to SEQ ID NO:42; (g) a LukA polypeptide with an L61R amino acid substitution corresponding to SEQ ID NO:15 and a LukB polypeptide with a K12A/K19A/R23A triple amino acid substitution corresponding to SEQ ID NO:42; (h) a LukA polypeptide with an L61R amino acid substitution corresponding to SEQ ID NO: 15 and a LukB-HlgB polypeptide of SEQ ID NO: 42; (i) a LukA polypeptide with a D39A amino acid substitution corresponding to SEQ ID NO: 15 and a LukB polypeptide with an E112A amino acid substitution corresponding to SEQ ID NO: 42; (j) a LukA polypeptide with a D39A amino acid substitution corresponding to SEQ ID NO: 15 and a LukB polypeptide with a K12A/K19A/R23A triple amino acid substitution corresponding to SEQ ID NO:42; (k) a LukA polypeptide with a D39A amino acid substitution corresponding to SEQ ID NO:15 and a LukB polypeptide with a K12A/K19A/R23A triple amino acid substitution corresponding to SEQ ID NO:42; (1) a LukA polypeptide with a D39A amino acid substitution corresponding to SEQ ID NO: 15 and a LukB polypeptide with an R23E amino acid substitution corresponding to SEQ ID NO:42; (m) a LukA polypeptide with a D39A amino acid substitution corresponding to SEQ ID NO: 15 and a LukB polypeptide with a K218A amino acid substitution corresponding to SEQ ID NO:42; (n) a LukA polypeptide with a D39R amino acid substitution corresponding to SEQ ID NO: 15 and a LukB polypeptide with an E112A amino acid substitution corresponding to SEQ ID NO:42; (o) a LukA polypeptide with a D39R amino acid substitution corresponding to SEQ ID NO: 15 and a LukB polypeptide with an R23E amino acid substitution corresponding to SEQ ID NO:42; (p) a LukA polypeptide with a D39R amino acid substitution corresponding to SEQ ID NO: 15 and a LukB polypeptide with a K218A amino acid substitution corresponding to SEQ ID NO:42; (q) a LukA polypeptide with a D39R amino acid substitution corresponding to SEQ ID NO: 15 and a LukB polypeptide with a K12A/K19A/R23A triple amino acid substitution corresponding to SEQ ID NO:42; (r) a LukA polypeptide with a D39R amino acid substitution corresponding to SEQ ID NO: 15 and a LukB polypeptide with a K12A/K19A/R23A triple amino acid substitution corresponding to SEQ ID NO:42; (s) a LukA polypeptide with a D197K amino acid substitution corresponding to SEQ ID NO:15 and a LukB polypeptide with an R23A amino acid substitution corresponding to SEQ ID NO:42; (t) a LukA polypeptide with a D197K amino acid substitution corresponding to SEQ ID NO:15 and a LukB polypeptide with an R23E amino acid substitution corresponding to SEQ ID NO:42; or (u) a LukA polypeptide with a K138A amino acid substitution corresponding to SEQ ID NO: 15 and a LukB polypeptide with a K218A amino acid substitution corresponding to SEQ ID NO: 42.

In another general aspect, the invention relates to one or more isolated nucleic acids encoding a Staphylococcus aureus protein A (SpA) variant polypeptide and a mutant staphylococcal leukocidin subunit polypeptide (i.e., a mutant LukA polypeptide, a mutant LukB polypeptide, and/or a mutant LukAB dimer polypeptide) of the invention. The isolated nucleic acid can encode a SpA variant polypeptide and an isolated mutant staphylococcal leukocidin subunit polypeptide comprising, consisting of, or consisting essentially of a wild-type staphylococcal LukA subunit, a wild-type staphylococcal LukB subunit, or a wild-type staphylococcal LukAB dimer, except for having one or more mutations as described herein, which reduce toxicity of the mutant leukocidin subunit relative to the corresponding wild-type leukocidin subunit. In certain aspects, the substitutions, deletions, or a combination thereof do not significantly reduce the immunogenicity of the mutant LukA subunit, mutant LukB subunit, or the mutant LukAB dimer relative to the corresponding wild-type leukocidin subunit or dimer. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding polypeptides or fragments thereof of the invention can be altered without changing the amino acid sequences of the proteins.

In another general aspect, the invention relates to a vector comprising one or more isolated nucleic acids encoding a SpA variant polypeptide and a mutant staphylococcal leukocidin subunit polypeptide (i.e., a mutant LukA polypeptide, a mutant LukB polypeptide, and/or a mutant LukAB dimer polypeptide) of the invention. Any vector known to those skilled in the art in view of the present disclosure can be used, such as a plasmid, a cosmid, a phage vector, a viral vector, a self-replicating RNA, or a replicon. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention. Such techniques are well known to those skilled in the art in view of the present disclosure.

Once an expression vector is selected, the polynucleotide as described herein can be cloned downstram of the promoter, for example, in a polylinker region. The vector is transformed into an appropriate bacterial strain, and DNA is prepared using standard techniques. The orientation and DNA sequence of the polypeptide as well as other elements included in the vector are confirmed using restriction mapping, DNA sequence analysis, and/or PCR analysis. Bacterial cells harboring the correct vector can be stored as cell banks.

In another general aspect, the invention relates to a host cell comprising one or more isolated nucleic acids encoding a SpA variant polypeptide and a mutant staphylococcal leukocidin subunit polypeptide (i.e., a mutant LukA polypeptide, a mutant LukB polypeptide, and/or a mutant LukAB dimer polypeptide) of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-KI cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a SpA variant polypeptide and a mutant staphylococcal leukocidin subunit polypeptide (i.e., a mutant LukA polypeptide, a mutant LukB polypeptide, and/or a mutant LukAB dimer polypeptide) of the invention, comprising culturing a cell comprising one or more nucleic acids encoding the SpA variant polypeptide and the mutant staphylococcal leukocidin subunit polypeptide under conditions to produce a SpA variant polypeptide and a mutant staphylococcal leukocidin subunit polypeptide of the invention, and recovering the SpA variant polypeptide and the mutant staphylococcal leukocidin subunit polypeptide from the cell or cell culture (e.g., from the supernatant). Expressed SpA variant polypeptides and mutant staphylococcal leukocidin subunit polypeptides (i.e., mutant LukA polypeptides, mutant LukB polypeptides, and/or mutant LukAB dimer polypeptides) can be harvested from the cells and purified according to conventional techniques known in the art and as described herein. Methods for the production of mutant LukAB dimer polypeptides are known in the art, see, e.g., DuMont et al., Infection and Immunity 82(3):1268-76 (2014); Kailasan et al., Toxins 11(6):339 (2019).

Staphylococcal Protein A (SpA)

“Protein A” and “SpA,” as used herein, can be used interchangeably, and refer to a cell wall anchored surface protein of S. aureus, which functions to provide for bacterial evasion from the innate and adaptive immune responses of the host to be infected. Protein A can bind immunoglobulins at their Fc portion, can interact with the VH3 domain of B cell receptors in appropriately stimulating B cell proliferation and apoptosis, can bind von Willebrand factor A1 domains to activate intracellular clotting, and can also bind to the TNF Receptor-1 to contribute to the pathogenesis of staphylococcal pneumonia.

All S. aureus strains express the structural gene for Protein A (spa) (Jensen (1958); Said-Salim et al., (2003)), a well characterized virulence factor whose cell wall anchored surface protein product (SpA) encompasses five highly homologous immunoglobulin binding domains designated E, D, A, B, and C (Sjodahl, (1977)). The immunoglobulin domains display ˜80% identity at the amino acid level, are 56 to 61 residues in length, and are organized as tandem repeats (Uhlen et al., (1984)). Each of the immunoglobulin binding domains is composed of anti-parallel α-helices that assemble into a three helix bundle and bind the Fc domain of immunoglobulin G (IgG) (Deisenhofer, (1981); Deisenhofer et al., (1978)), the VH3 heavy chain (Fab) of IgM (Graille et al., (2000)), the von Willebrand factor at its A1 domain (O'Seaghdha et al., (2006)), and the tumor necrosis factor α (TNF-α) receptor 1 (TNFR1) (Gomez et al., (2006)).

SpA impedes neutrophil phagocytosis of staphylococci through binding the Fc component of IgG (Jensen, (1958); Uhlen et al., (1984)). Additionally, SpA is able to activate intravascular clotting via binding to von Willebrand factor A1 domains (Hartleib et al., (2000)). Plasma proteins, such as fibrinogen and fibronectin act as bridges between staphylococci (ClfA and ClfB) and the platelet integrin GPIIb/IIIa (O'Brien et al., (2002)), an activity that is supplemented through SpA association with vWF A1, which allows staphylococci to capture platelets via the GPIb-α platelet receptor (Foster, (2005); O'Seaghdha et al., (2006)). SpA also binds TNFR1, and this interaction contributes to the pathogenesis of staphylococcal pneumonia (Gomez et al., (2004)). SpA activates proinflammatory signaling through TNFR1 mediated activation of TRAF2, the p38/c-Jun kinase, mitogen activated protein kinase (MAPK), and the Rel-transcription factor NF-κB. SpA binding further induces TNFR1 shedding, an activity that appears to require the TNF-converting enzyme (TACE) (Gomez et al., (2007)). Each of the disclosed activities are mediated through the five IgG binding domains and can be perturbed by the same amino acid substitutions, initially defined by their requirement for the interaction between Protein A and human IgG1 (Cedergren et al., (1993)).

SpA also functions as a B cell superantigen by capturing the Fab region of VH3 bearing IgM, the B cell receptor (Gomez et al., (2007); Goodyear et al., (2003); Goodyear and Silverman (2004); Roben et al., (1995)). Following intravenous challenge, staphylococcal SpA mutations show a reduction in staphylococcal load in organ tissues and dramatically diminished ability to form abscesses. During infection with wild-type S. aureus, abscesses are formed within forty-eight hours and are detectable by light microscopy of hematoxylin-cosin stained, thin-sectioned kidney tissue, initially marked by an influx of polymorphonuclear leukocytes (PMNs). On day 5 of the infection, abscesses increase in size and enclosed a central population of staphylococci, surrounded by a layer of eosinophilic, amorphous material and a large cuff of PMNs. Histopathology revealed a massive necrosis of PMNs in proximity to the staphylococcal nidus at the center of abscess lesions as well as a mantle of healthy phagocytes. A rim of necrotic PMNs at the periphery of abscess lesions, bordering eosinophilic pseudocapsule that separated healthy renal tissue from the infections lesion, was also observed. Staphylococcal variants lacking SpA are unable to establish histopathology features of abscesses and are cleared during infection.

As disclosed herein, the terms “Protein A variant,” “SpA variant,” “Protein A variant polypeptide,” and “SpA variant polypeptide” refer to a polypeptide including a SpA IgG domain having at least one amino acid substitution that disrupts the binding to Fc and VH3. In certain embodiments, the SpA variant polypeptide includes a variant D domain, as well as variants and fragments thereof that are non-toxic and stimulate an immune response against staphylococcus bacteria Protein A and/or bacteria expressing the same.

Described herein are SpA variant polypeptides that no longer are able to bind to immunoglobulins, which thereby functions to eliminate the toxicity associated with a SpA polypeptide. The SpA variant polypeptides are non-toxic and stimulate humoral immune responses to protect against staphylococcal infection and disease.

In certain embodiments, the SpA variant polypeptide is a full-length SpA variant comprising a variant A, B, C, D, and/or E domain. In certain embodiments, the SpA variant polypeptide comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO:60 or 61. In certain embodiments, the SpA variant polypeptide comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO:54.

In certain embodiments, the SpA variant polypeptide comprises a fragment of the full-length SpA polypeptide. The SpA variant polypeptide fragment can comprise 1, 2, 3, 4, 5, or more IgG binding domains. The IgG binding domains can, for example, be 1, 2, 3, 4, 5, or more variant A, B, C, D, and/or E domains.

In certain embodiments, the SpA variant polypeptide comprises 1, 2, 3, 4, 5, or more variant A domains. In certain embodiments, the SpA variant polypeptide comprises 1, 2, 3, 4, 5, or more variant B domains. In certain embodiments, the SpA variant polypeptide comprises 1, 2, 3, 4, 5, or more variant C domains. In certain embodiments, the SpA variant polypeptide comprises 1, 2, 3, 4, 5, or more variant D domains. In certain embodiments, the SpA variant polypeptide comprises 1, 2, 3, 4, 5, or more variant E domains.

The variant A domain can, for example, comprise an amino acid sequence of SEQ ID NO:55. The variant B domain can, for example, comprise an amino acid sequence of SEQ ID NO:56. The variant C domain can, for example, comprise an amino acid sequence of SEQ ID NO:57. The variant D domain can, for example, comprise an amino acid sequence of SEQ ID NO:58. The variant E domain can, for example, comprise an amino acid sequence of SEQ ID NO:59.

In certain embodiments, the SpA variant polypeptide can comprise a variant A, B, C, D, and E domain, which can comprise an amino acid sequence having at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:55, SEQ ID NO:56, SEQ ID NO:57, SEQ ID NO:58, and SEQ ID NO:59, respectively.

In certain embodiments, the SpA variant polypeptide can comprise a variant A domain comprising a substitution at amino acid position 7, 8, 34, and/or 35 of SEQ ID NO:55. In certain embodiments, the SpA variant polypeptide can comprise a variant B domain comprising a substitution at amino acid position 7, 8, 34, and/or 35 of SEQ ID NO:56. In certain embodiments, the SpA variant polypeptide can comprise a variant C domain comprising a substitution at amino acid position 7, 8, 34, and/or 35 of SEQ ID NO:57. In certain embodiments, the SpA variant polypeptide can comprise a variant D domain comprising a substitution at amino acid position 9, 10, 36, and/or 37 of SEQ ID NO:58. In certain embodiments, the SpA variant polypeptide can comprise a variant E domain comprising a substitution at amino acid position 6, 7, 33, and/or 34 of SEQ ID NO:59. Amino acid substitutions in variant A, B, C, D, and/or E domains are described in WO2011/005341.

In certain embodiments, the SpA variant polypeptide comprises one or more amino acid substitutions in an IgG Fc binding sub-domain of the SpA domain D, or at a corresponding amino acid position in the other IgG domains. The one or more amino acid substitutions can disrupt or decrease the binding of the SpA variant polypeptide to the IgG Fc. In certain embodiments, the SpA variant polypeptide further comprises one or more amino acid substitutions in a V_{H}3 binding sub-domain of the SpA domain D, or at a corresponding amino acid position in the other IgG domains. The one or more amino acid substitutions can disrupt or decrease binding to V_{H}3.

In certain embodiments, the amino acid residues F5, Q9, Q10, S11, F13, Y14, L17, N28, 131, and/or K35 of the IgG Fc binding sub-domain of SpA D domain of SEQ ID NO:58 are modified or substituted such that binding to IgG Fc is reduced or eliminated.

In certain embodiments, the amino acid residues Q26, G29, F30, S33, D36, D37, Q40, N43, and/or E47 of the V_{H}3 binding sub-domain of SpA D domain of SEQ ID NO:58 are modified or substituted such that binding to V_{H}3 is reduced or eliminated.

The corresponding modifications can be incorporated in SpA domain A, B, C, and/or E. Corresponding positions are defined by an alignment of the SpA domain D with SpA domain A, B, C, and/or E to determine the corresponding residues from SpA domain D with SpA domain A, B, C, and/or E.

In certain embodiments, the SpA variant polypeptide comprises (a) one or more amino acid substitutions in an IgG Fc binding sub-domain of the SpA domain D, or at a corresponding amino acid position in the other IgG domains; and (b) one or more amino acid substitutions in a V_{H}3 binding sub-domain of the SpA domain D, or at a corresponding amino acid position in the other IgG domains. The one or more amino acid substitutions reduces the binding of the SpA variant polypeptide to an IgG Fc and V_{H}3 such that the SpA variant polypeptide has reduced or eliminated toxicity in a host organism.

In certain embodiments, the SpA variant polypeptide comprises 1, 2, 3, 4, 5, 6, 7, 8, 9, 10, or more variant D domains. The variant D domains can comprise 1, 2, 3, 4, 5, 6, 7, 8, 9, 10, or more amino acid residue substitutions or modifications. The amino acid residue substitutions or modifications can, for example, occur at amino acid residue F5, Q9, Q10, S11, F13, Y14, L17, N28, 131, and/or K35 of the IgG Fc binding sub-domain of the SpA domain D (SEQ ID NO:58) and/or at amino acid residue Q26, G29, F30, S33, D36, D37, Q40, N43, and/or E47 of the V_{H}3 binding sub-domain of the SpA domain D (SEQ ID NO:58). In certain embodiments, the amino acid residue substitution or modification is at amino acid residues Q9 and Q10 of SEQ ID NO:58. In certain embodiments, the amino acid residue substitution or modification is at amino acid residues D36 and D37 of SEQ ID NO:58. Amino acid substitutions in variant A, B, C, D, and/or E domains are described in WO2011/005341, which is incorporated by reference herein in its entirety.

In certain embodiments, the SpA variant polypeptide comprises an amino acid sequence having at least 75%, at least 80%, at least 85%, at least 90%, at least 91%, at least 92%, at least 93%, at least 94%, at least 95%, at least 96%, at least 97%, at least 98%, or at least 99% identity to SEQ ID NO:72 and/or comprises a fragment of at least n consecutive amino acids of SEQ ID NO:72, wherein n is at least 7, at least 8, at least 10, at least 20, at least 30, at least 40, at least 50, at least 75, at least 100, at least 125, at least 150, at least 175, at least 200, at least 225, at least 250, at least 275, at least 300, at least 325, at least 350, at least 375, at least 400, or at least 425 amino acids. In certain embodiments, the SpA variant polypeptide can comprise a deletion of one or more amino acids from the carboxy (C)-terminus (e.g., at least 1, 2, 3, 4, 5, 10, 15, 20, 25, 30, or 35 amino acids) and/or a deletion of one or more amino acids from the amino (N)-terminus (e.g., at least 1, 2, 3, 4, 5, 10, 15, 20, 25, 30, or 35 amino acids) of SEQ ID NO:72. In certain embodiments, the final 35 C-terminal amino acids are deleted. In certain embodiments, the first 36 N-terminal amino acids are deleted. In certain embodiments, the SpA variant polypeptide comprises amino acids 37 to 325 of SEQ ID NO:72.

In certain embodiments, the SpA variant polypeptide comprising all five SpA Ig-binding domains, which arranged from the N- to C-terminus comprise in order the E domain, D domain, A domain, B domain, and C domain. In certain embodiments, the SpA variant polypeptide comprises 1, 2, 3, or 4 of the natural A, B, C, D, and/or E domains. In embodiments in which 1, 2, 3, or 4 of the natural domains are deleted, the SpA variant polypeptide can prevent the excessive B cell expansion and apoptosis which can occur if SpA functions as a B cell superantigen. In certain embodiments, the SpA variant polypeptide comprises only the SpA A domain. In certain embodiments, the SpA variant polypeptide comprises only the SpA B domain. In certain embodiments, the SpA variant polypeptide comprises only the SpA C domain. In certain embodiments, the SpA variant polypeptide comprises only the SpA D domain. In certain embodiments, the SpA variant polypeptide comprises only the SpA E domain.

In certain embodiments, the SpA variant polypeptide comprises mutations of at least one of eleven (11) dipeptide sequence repeats relative to SEQ ID NO:72 (e.g., a QQ dipeptide repeat and/or a DD dipeptide repeat). By way of an example, the SpA variant polypeptide comprises the amino acid sequence of SEQ ID NO:73, wherein the XX dipeptide repeats at amino acid positions 7 and 8, 34 and 35, 60 and 61, 68 and 69, 95 and 96, 126 and 127, 153 and 154, 184 and 185, 211 and 212, 242 and 243, and 269 and 270 can be mutated to reduce the affinity of the SpA variant polypeptide for immunoglobulins. Useful dipeptide substitutions for a Gln-Gln (QQ) dipeptide can include, but are not limited to, a Lys-Lys (KK), an Arg-Arg (RR), an Arg-Lys (RK), a Lys-Arg (KR), an Ala-Ala (AA), a Ser-Ser (SS), a Ser-Thr (ST), and a Thr-Thr (TT) dipeptide. Preferably, a QQ dipeptide is substituted with a KR dipeptide. Useful dipeptide substitutions for an Asp-Asp (DD) dipeptide can include, but are not limited to, an Ala-Ala (AA), a Lys-Lys (KK), an Arg-Arg (RR), a Lys-Arg (KR), a His-His (HH), and a Val-Val (VV) dipeptide. The dipeptide substitutions can, for example, decrease the affinity of the SpA variant polypeptide for the Fc portion of the human IgG and the Fab portion of V_{H3}-containing human B cell receptors.

Thus, in certain embodiments, the SpA variant polypeptide can comprise SEQ ID NO:78, wherein one or more, preferably all 11 of the XX dipeptides are substituted with amino acids that differ from the corresponding dipeptides of SEQ ID NO:72. In certain embodiments, the SpA variant polypeptide comprises SEQ ID NO:79, wherein the amino acid doublet at positions 60 and 61 are Lys and Arg (K and R), respectively. In certain embodiments, the SpA variant polypeptide comprises SEQ ID NO:80 or SEQ ID NO:81. In certain embodiments, the SpA variant polypeptide comprises SEQ ID NO:75, wherein a preferred example of SEQ ID NO:75 is SEQ ID NO:76 or SEQ ID NO:77 (SEQ ID NO:77 is SEQ ID NO:76 with an N-terminal methionine).

In certain embodiments, the SpA variant polypeptide N-terminus can comprise a deletion of the first 36 amino acids of SEQ ID NO:72, and the C-terminus can comprise a deletion of the last 35 amino acids of SEQ ID NO:72. The SpA variant polypeptide comprising a N-terminal deletion of 36 amino acids of SEQ ID NO:72 and a C-terminal deletion of 35 amino acids of SEQ ID NO:72 can further comprise a deletion of the fifth Ig-binding domain (i.e., downstream of Lys-327 of SEQ ID NO:72). This SpA variant can comprise the amino acid sequence of SEQ ID NO:73, wherein the XX dipeptides can be substituted with amino acids, such that the amino acids differ from the corresponding dipeptide sequences in SEQ ID NO:72. In certain embodiments, the SpA variant polypeptide comprises SEQ ID NO:74.

In certain embodiments, as noted above, a SpA variant polypeptide can comprise 1, 2, 3, or 4 of the natural A, B, C, D, and/or E domains, e.g., comprise only the SpA E domain but not the D, A, B, or C. Thus, the SpA variant polypeptide can comprise a variant SpA E domain, wherein the SpA E domain comprises a substitution in at least one amino acid of SEQ ID NO:83. The substitution can, for example, be at amino acid positions 60 and 61 of SEQ ID NO:83. In certain embodiments, the SpA variant polypeptide can comprise SEQ ID NO:79, SEQ ID NO:80, SEQ ID NO:81, or SEQ ID NO:82. In certain embodiments, the SpA variant polypeptide can comprise SEQ ID NO:79, SEQ ID NO:80, SEQ ID NO:81, or SEQ ID NO:82 with at least one amino acid substitution. SpA variant polypeptides are described in WO2015/144653, which is incorporated by reference herein in its entirety.

In certain embodiments, the SpA variant polypeptide comprises an amino acid substitution at amino acids 43Q, 44Q, 96Q, 97Q, 162Q, 163Q, 220Q, 221Q, 278Q, and 279Q of SEQ ID NO:84. The amino acid substitution at amino acids 43Q, 44Q, 96Q, 97Q, 162Q, 163Q, 220Q, 221Q, 278Q, and 279Q of SEQ ID NO:84 can, for example, be a lysine (K) or an arginine (R) substitution. In certain embodiments, the SpA variant polypeptide comprises an amino acid substitution at amino acids 70D, 71D, 131D, 132D, 189D, 190D, 247D, 248D, 305D, and 306D of SEQ ID NO:84. The amino acid substitution at amino acids 70D, 71D, 131D, 132D, 189D, 190D, 247D, 248D, 305D, and 306D of SEQ ID NO:84 can, for example, be an alanine (A) or a valine (V) substitution. In certain embodiments, the SpA variant polypeptide can be selected from SEQ ID NO:85, SEQ ID NO:86, SEQ ID NO:87, and SEQ ID NO:88. SpA variant polypeptides are described in US2016/0304566, which is incorporated by referenced herein in its entirety.

In certain embodiments, the variant A domain can, for example, comprise an amino acid sequence of SEQ ID NO:62 or 67. The variant B domain can, for example, comprise an amino acid sequence of SEQ ID NO:63 or 68. The variant C domain can, for example, comprise an amino acid sequence of SEQ ID NO:64 or 69. The variant D domain can, for example, comprise an amino acid sequence of SEQ ID NO:66 or 71. The variant E domain can, for example, comprise an amino acid sequence of SEQ ID NO:65 or 70.

In a preferred embodiment, the variant A domain can, for example, comprise an amino acid sequence of SEQ ID NO:62. The variant B domain can, for example, comprise an amino acid sequence of SEQ ID NO:63. The variant C domain can, for example, comprise an amino acid sequence of SEQ ID NO:64. The variant D domain can, for example, comprise an amino acid sequence of SEQ ID NO: 66. The variant E domain can, for example, comprise an amino acid sequence of SEQ ID NO: 65.

In certain embodiments, the SpA variant polypeptide can comprise a variant A, B, C, D, and E domain, which can comprise an amino acid sequence having at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:62 or 67, SEQ ID NO:63 or 68, SEQ ID NO:64 or 69, SEQ ID NO:66 or 71, and SEQ ID NO:65 or 70, respectively.

In certain embodiments, the SpA variant polypeptide can comprise a variant D domain comprising a substitution at amino acid position 9, 10, and/or 33 of SEQ ID NO:58.

In certain embodiments, the SpA variant polypeptide comprises (i) lysine substitutions for glutamine amino acid residues in each of SpA A-E domains corresponding to positions 9 and 10 of SpA D domain (SEQ ID NO:58); and (ii) a glutamate substitution for a serine amino acid residue in each of SpA A-E domains corresponding to position 33 of SpA D domain (SEQ ID NO:58). The SpA variant polypeptide does not, relative to a negative control, detectably crosslink IgG and IgE in blood and/or activate basophils. By not detectably crosslinking IgG and IgE in blood and/or activating basophils, it is believed that the SpA variant polypeptide does not pose a significant safety or toxicity issue to human patients or does not pose a significant risk of anaphylactic shock in a human patient.

In certain embodiments, the K_{A} binding affinity for V_{H}3 from human IgG is reduced as compared to a SpA variant polypeptide (SpA_{KK}AA) consisting of lysine substitutions for glutamine residues in each of SpA A-E domains corresponding to positions 9 and 10 of SpA D domain (SEQ ID NO:58) and alanine substitutions for aspartic acid in SpA A-E domains corresponding to positions 36 and 37 of SpA D domain (SEQ ID NO:58). The SpA variant polypeptide consisting of lysine substitutions for glutamine residues in each of domains A-E corresponding to positions 9 and 10 in domain D and alanine substitutions for aspartic acid in domains A-E corresponding to positions 36 and 37 of domain D, is used as a comparator and is named SpA_{KKAA}. The SpA_{KKAA} variant polypeptide has an amino acid sequence of SEQ ID NO:54. In certain embodiments, the SpA variant polypeptide has a K_{A} binding affinity for V_{H}3 form human IgG that is reduced by at least two-fold (2-fold) as compared to SpA_{KKAA}. In certain embodiments, the SpA variant polypeptide has a K_{A} binding affinity for V_{H}3 from human IgG that is reduced at least 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3-fold or more or any value in between as compared to SpA_{KKAA}. In certain embodiments, the SpA variant polypeptide has a K_{A} binding affinity for V_{H}3 from human IgG that is reduced at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300% or more or any value in between as compared to SpA_{KKAA}. In certain embodiments, the SpA variant polypeptide has a K_{A} binding affinity for V_{H}3 from human IgG that is less than about 1×10⁵ M⁻¹. In certain embodiments, the SpA variant polypeptide has a K_{A} binding affinity for V_{H}3 from human IgG that is less than about 3, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1×10⁵ M⁻¹ or any value in between. In certain embodiments, the SpA variant polypeptide does not have substitutions in any of the SpA A-E domains corresponding to positions 36 and 37 of SpA D domain (SEQ ID NO:58). In certain embodiments, the SpA variant polypeptide of the present invention comprises SEQ ID NO:66 or 71. In certain embodiments, the SpA variant polypeptide of the invention comprises SEQ ID NO:60 or 61. In a preferred embodiment, the SpA variant polypeptide of the invention comprises SEQ ID NO: 60.

In certain embodiments, the SpA variant polypeptide comprises (i) lysine substitutions for glutamine amino acid residues in each of SpA A-E domains corresponding to positions 9 and 10 of SpA D domain (SEQ ID NO:58); and (ii) a threonine substitution for a serine amino acid residue in each of SpA A-E domains corresponding to position 33 of SpA D domain (SEQ ID NO:58). The SpA variant polypeptide does not, relative to a negative control, detectably crosslink IgG and IgE in blood and/or activate basophils. By not detectably crosslinking IgG and IgE in blood and/or activating basophils, it is believed that the SpA variant polypeptide does not pose a significant safety or toxicity issue to human patients or does not pose a significant risk of anaphylactic shock in a human patient.

In certain embodiments, the K_{A} binding affinity for V_{H}3 from human IgG is reduced as compared to a SpA variant polypeptide (SpA_{KK}AA) consisting of lysine substitutions for glutamine residues in each of SpA A-E domains corresponding to positions 9 and 10 of SpA D domain (SEQ ID NO:58) and alanine substitutions for aspartic acid in SpA A-E domains corresponding to positions 36 and 37 of SpA D domain (SEQ ID NO:58). In certain embodiments, the SpA variant polypeptide has a K_{A} binding affinity for V_{H}3 form human IgG that is reduced by at least two-fold (2-fold) as compared to SpA_{KKAA}. In certain embodiments, the SpA variant polypeptide has a K_{A} binding affinity for V_{H}3 from human IgG that is reduced at least 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3-fold or more or any value in between as compared to SpA_{KKAA}. In certain embodiments, the SpA variant polypeptide has a K_{A} binding affinity for V_{H}3 from human IgG that is reduced at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300% or more or any value in between as compared to SpA_{KKAA}. In certain embodiments, the SpA variant polypeptide has a K_{A} binding affinity for V_{H}3 from human IgG that is less than about 1×10⁵ M⁻¹. In certain embodiments, the SpA variant polypeptide has a K_{A} binding affinity for V_{H}3 from human IgG that is less than about 3, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1×10⁵ M⁻¹ or any value in between. In certain embodiments, the SpA variant polypeptide does not have substitutions in any of the SpA A-E domains corresponding to positions 36 and 37 of SpA D domain (SEQ ID NO:58). In certain embodiments, the SpA variant polypeptide comprises SEQ ID NO:60.

In certain embodiments, the SpA variant polypeptide comprises one or more amino acid substitutions in an IgG Fc binding sub-domain of the SpA domain D, or at a corresponding amino acid position in the other IgG domains. The one or more amino acid substitutions can disrupt or decrease the binding of the SpA variant polypeptide to the IgG Fc. In certain embodiments, the SpA variant polypeptide further comprises one or more amino acid substitutions in a V_{H}3 binding sub-domain of the SpA domain D, or at a corresponding amino acid position in the other IgG domains. The one or more amino acid substitutions can disrupt or decrease binding to V_{H}3.

The corresponding modifications can be incorporated in SpA domain A, B, C, and/or E. Corresponding positions are defined by an alignment of the SpA domain D with SpA domain A, B, C, and/or E to determine the corresponding residues from SpA domain D with SpA domain A, B, C, and/or E.

In certain embodiments, the SpA variant polypeptide comprises (a) one or more amino acid substitutions in an IgG Fc binding sub-domain of the SpA domain D, or at a corresponding amino acid position in the other IgG domains; and (b) one or more amino acid substitutions in a V_{H3} binding sub-domain of the SpA domain D, or at a corresponding amino acid position in the other IgG domains. The one or more amino acid substitutions reduces the binding of the SpA variant polypeptide to an IgG Fc and V_{H}3 such that the SpA variant polypeptide has reduced or eliminated toxicity in a host organism.

Additional Staphylococcus Peptides

In certain embodiments, the immunogenic composition comprising a Staphylococcus aureus protein A (SpA) variant polypeptide and/or a mutant staphylococcal leukocidin subunit polypeptide (e.g., the staphylococcal LukA polypeptide, the staphylococcal LukB polypeptide, and/or the staphylococcal LukAB dimer polypeptide), as described herein, further comprises at least one or more staphylococcal antigens or immunogenic fragments thereof selected from the group consisting of CP5, CP8, Eap, Ebh, Emp, EsaB, EsaC, EsxA, EsxB, EsxAB(fusion), SdrC, SdrD, SdrE, IsdA, IsdB, IsdC, ClfA, ClfB, Coa, Hla (e.g., H35 mutants, or H35L/H48L), mHla, MntC, rTSST-1, rTSST-1v, SasF, vWbp, and vWh. Additional staphylococcal antigens that can be included in the immunogenic composition can include, but are not limited to, vitronectin binding protein (WO2001/60852), Aaa (GenBank CAC80837), Aap (GenBank AJ249487), Ant (GenBank NP_372518), autolysin glucosaminidase, autolysin amidase, Can, collagen binding protein (U.S. Pat. No. 6,288,214), CsalA, EFB (FIB), Elastin binding protein (EbpS), EPB, FbpA, fibrinogen binding protein (U.S. Pat. No. 6,008,341), Fibronectin binding protein (U.S. Pat. No. 5,840,846), FhuD, FhuD2, FnbA, FnbB, GehD (US 2002/0169288), HarA, HBP, Immunodominant ABC transporter, IsaA/PisA, laminin receptor, Lipase GehD, MAP, Mg2+ transporter, MHC II analog (U.S. Pat. No. 5,648,240), MRPII, NPase, RNA III activating protein (RAP), SasA, SasB, SasC, SasD, SasK, SBI, SdrF (WO 2000/12689), SdrG (WO 2000/12689), SdrH (WO 2000/12689), SEA exotoxins (WO 2000/02523), SEB exotoxins (WO 2000/02523), mSEB, SitC and Ni ABC transporter, SitC/MntC/saliva binding protein (U.S. Pat. No. 5,801,234), SsaA, SSP-1, SSP-2, Spa5 (US2016/0304566), SpA_{KKAA} (WO2011/005341, WO2015/144653, WO 2015/144691), SpAkR (WO2015/144653), Sta006, and/or Sta011.

Additional staphylococcal antigens that can be included in the immunogenic composition can include, but are not limited to, a mutant LukS-PV subunit, a LukF-PV subunit, a mutant Gamma hemolysin A, a mutant Gamma hemolysin B, a mutant Gamma hemolysin (Hlg), Panton-Valentine Leukocidins (PVL), LukE, LukD, LukED dimers or any combination thereof.

Virulent encapsulated strains of S. aureus carry capsule polysaccharide type 5 (CP5) or type 8 (CP8) (O'Riordan and Lee, Clin. Microbiol. Rev. 17(1):218-34 (2004) PMID: 14726462). Staphylococcal CP-based vaccines elicit antibodies that promote opsonophagocytic killing (OPK) of S. aureus (Karakawa et al., Infect. Immun. 56(5):1090-5 (1988) PMID: 3356460), and immunization has been shown to protect experimental animals against staphylococcal bacteremia, lethality, mastitis, osteomyelitis, and endocarditis (Cheng et al., Human Vaccines & Immunother. 13(7):1609-14 (2017); Kuipers et al., Micro. 162(7):1185-94 (2016)). CP5 and CP8 are composed of repeats of highly similar trisaccharides, which only differ in the linkage between their monosaccharides and O-acetylation. The immune response against CP5 and CP8 is considered serotype specific. However, it has been suggested that CP8-induced antibodies can be cross-reactive against CP5 strains, whereas CP5-induced antibodies are serotype specific (Park et al., Infect. Immun. 82(12):5049-55 (2014) PMID: 25245803). Capsular polysaccharides are T-independent immunogens and they are weakly immunogenic. To improve the immunogenicity of capsule polysaccharide it needs to be chemically or enzymaticatically covalently (or linked via high-affinity noncovalent linkages) linked to a carrier protein to make an artificial glycoprotein or glycoconjugate (Fattom et al., Infect. Immun. 61(3):1023-32 (1993) PMID: 8432585). By means of conjugation, a glycoconjugate will be formed that consists of a capsule polysaccharide and a carrier protein, such as, but not limited to, CRM197. CRM197 is a non-toxic mutant of diphtheria toxin having a single amino acid substitution of glutamic acid for glycine. CRM197 is a well-defined protein and functions as a carrier for polysaccharides and haptens making them immunogenic. It is utilized as a carrier protein in a number of approved conjugate vaccines for diseases such as meningitis and pneumococcal bacterial infections. CP5 and CP8 can be produced as a native antigen from S. aureus biomass or can be chemically synthesized. Other carrier proteins besides CRM197 can be used. The example of CRM197 is not considered to be limiting.

The additional staphylococcal antigen can be administered concurrently with the S. aureus protein A (SpA) variant polypeptide and/or the mutant staphylococcal leukocidin subunit polypeptide (e.g., the staphylococcal LukA polypeptide, the staphylococcal LukB polypeptide, and/or the staphylococcal LukAB dimer polypeptide). The staphylococcal antigen can be administered with the S. aureus protein A (SpA) variant polypeptide and/or the mutant staphylococcal leukocidin subunit polypeptide (e.g., the staphylococcal LukA polypeptide, the staphylococcal LukB polypeptide, and/or the staphylococcal LukAB dimer polypeptide) in the same immunogenic composition.

In certain embodiments, the SpA variant polypeptide and/or the mutant staphylococcal leukocidin subunit polypeptide described herein further comprises a heterologous amino acid sequence. The heterologous amino acid sequence can, for example, encode a peptide selected from the group consisting of a His-tag, a ubiquitin tag, a NusA tag, a chitin binding domain, a B-tag, a HSB-tag, green fluorescent protein (GFP), a calmodulin binding protein (CBP), a galactose-binding protein, a maltose binding protein (MBP) cellulose binding domains, an avidin/streptavidin/Strep-tag, trpE, chloramphenicol acetyltransferase (CAT), lacZ(β-galactosidase), a FLAG™ peptide, an S-tag, a T7-tag, a fragment thereof of a heterologous amino acid sequence, and a combination of two or more of said heterologous amino acid sequences. In certain embodiments, the heterologous amino acid sequence encodes an immunogen, a T-cell epitope, a B-cell epitope, a fragment thereof of a heterologous amino acid sequence, and a combination of two or more of said heterologous amino acid sequences.

In another general aspect, the invention relates to one or more isolated nucleic acids encoding the S. aureus protein A (SpA) variant polypeptides and/or the mutant staphylococcal leukocidin subunit polypeptides (e.g., the staphylococcal LukA polypeptide, the staphylococcal LukB polypeptide, and/or the staphylococcal LukAB dimer polypeptide) of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding the polypeptides of the invention can be altered without changing the amino acid sequences of the proteins.

In another general aspect, the invention relates to a vector comprising one or more isolated nucleic acids encoding a S. aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide) of the invention. The term “vector,” as used herein, refers to e.g., any of a number of nucleic acids into which a desired sequence can be inserted, e.g., by restriction and ligation, for transport between different genetic environments or for expression in a host cell. Nucleic acid vectors can be DNA or RNA. Vectors include, but are not limited to, plasmids, phage, phagemids, bacterial genomes, and virus genomes. A cloning vector is one which is able to replicate in a host cell, and which is further characterized by one or more endonuclease restriction sites at which the vector can be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host bacterium or just a single time per host before the host reproduces by mitosis. In the case of phage, replication can occur actively during a lytic phase or passively during a lysogenic phase. Certain vectors are capable of autonomous replication in a host cell into which they are introduced. Other vectors are integrated into the genome of a host cell upon introduction into the host cell, and thereby are replicated along with the host genome.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of a fusion peptide in the cell. Conventional cloning techniques or artificial gene synthesis can be used to generate a recombinant expression vector according to embodiments of the invention.

Once an expression vector is selected, the polynucleotide as described herein can be cloned downstram of the promoter, for example, in a polylinker region. The vector is transformed into an appropriate bacterial strain, and DNA is prepared using standard techniques. The orientation and DNA sequence of the polypeptide as well as other elements included in the vector are confirmed using restriction mapping, DNA sequence analysis, and/or PCR analysis. Bacterial cells harboring the correct vector can be stored as cell banks.

In another general aspect, the invention relates to a host cell comprising one or more isolated nucleic acids encoding a S. aureus protein A (SpA) variant polypeptide and/or a mutant staphylococcal leukocidin subunit polypeptide (e.g., the staphylococcal LukA polypeptide, the staphylococcal LukB polypeptide, and/or the staphylococcal LukAB dimer polypeptide) of the invention or a vector comprising an isolated nucleic acid encoding a S. aureus protein A (SpA) variant polypeptide and/or a mutant staphylococcal leukocidin subunit polypeptide (e.g., the staphylococcal LukA polypeptide, the staphylococcal LukB polypeptide, and/or the staphylococcal LukAB dimer polypeptide) of the invention. Any host cell known to those skilled in the art in view of the present disclosure can be used for recombinant expression of mutant polypeptides of the invention. In some embodiments, the host cells are E. coli TG1 or BL21 cells, CHO-DG44 or CHO-KI cells or HEK293 cells. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Host cells are genetically engineered (infected, transduced, transformed, or transfected) with vectors of the disclosure. Thus, one aspect of the invention is directed to a host cell comprising a vector which contains the polynucleotide as described herein. The engineered host cell can be cultured in conventional nutrient media modified as appropriate for activating promoters, selecting transformants, or amplifying the polynucleotides. The culture conditions, such as temperature, pH and the like, are those previously used with the host cell selected for expression, and will be apparent to the ordinarily skilled artisan. The term “transfect,” as used herein, refers to any procedure whereby eukaryotic cells are induced to accept and incorporate into their genome isolated DNA, including but not limited to DNA in the form of a plasmid. The term “transform,” as used herein, refers to any procedure whereby bacterial cells are induced to accept and incorporate their genome isolated DNA, including, but not limited to DNA in the form of a plasmid.

In another general aspect, the invention relates to a method of producing a S. aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide) of the invention, comprising culturing a cell comprising one or more nucleic acids encoding a S. aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide) under conditions to produce the S. aureus protein A (SpA) variant polypeptide and the mutant staphylococcal leukocidin polypeptide of the invention, and recovering the polypeptide from the cell or cell culture (e.g., from the supernatant). Expressed polypeptides can be harvested from the cells and purified according to conventional techniques known in the art and as described herein.

Immunogenic Compositions

In another general aspect, the invention relates to an immunogenic composition, comprising a S. aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide) of the invention and a pharmaceutically acceptable carrier. The term “immunogenic composition” relates to any pharmaceutical composition comprising an antigen, e.g., a microorganism or a component thereof, which can be used to elicit an immune response in a subject. Isolated S. aureus protein A (SpA) variant polypeptides and isolated mutant staphylococcal leukocidin subunit polypeptides (e.g., the staphylococcal LukA polypeptide, the staphylococcal LukB polypeptide, and/or the staphylococcal LukAB dimer polypeptide) of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polypeptide pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms may be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, sublingually, or intradernally.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

Adjuvant

As used herein, the term “adjuvant” refers to a compound that when administered in conjunction with or as part of a composition of the invention augments, enhances and/or boosts the immune response to the LukA polypeptides, the LukB polypeptides, the LukAB dimer polypeptides, and/or the SpA variant polypeptides, but when the adjuvant compound is administered alone does not generate an immune response to the LukA polypeptides, the LukB polypeptides, the LukAB dimer polypeptides, and/or the SpA variant polypeptides. Adjuvants can enhance an immune response by several mechanisms including, e.g., lymphocyte recruitment, stimulation of B and/or T cells, and stimulation of antigen presenting cells.

The vaccine combinations of the invention (e.g., the immunogenic compositions comprising the LukA polypeptides, the LukB polypeptides, the LukAB dimer polypeptides, the SpA variant polypeptides, and/or polynucleotides, DNA or RNA, or viral vectors encoding the same) comprise, or are administered in combination with, an adjuvant. The adjuvant for administration in combination with an immunogenic composition of the invention can be administered before, concomitantly with, or after administration of the immunogenic compositions.

Specific examples of adjuvants include, but are not limited to, aluminum salts (alum) (such as aluminum hydroxide, aluminum phosphate, aluminum sulfate, and aluminum oxide, including nanoparticles comprising alum or nanoalum formulations), calcium phosphate (e.g. Masson J D et al, 2017, Expert Rev Vaccines 16: 289-299), monophosphoryl lipid A (MPL) or 3-de-O-acylated monophosphoryl lipid A (3D-MPL) (see e.g., United Kingdom Patent GB2220211, EP0971739, EP1194166, U.S. Pat. No. 6,491,919), AS01, AS02, AS03 and AS04 (all GlaxoSmithKline; see e.g. EP1126876, U.S. Pat. No. 7,357,936 for AS04, EP0671948, EP0761231, U.S. Pat. No. 5,750,110 for AS02), imidazopyridine compounds (see WO2007/109812), imidazoquinoxaline compounds (see WO2007/109813), delta-inulin (e.g. Petrovsky N and PD Cooper, 2015, Vaccine 33: 5920-5926), STING-activating synthetic cyclic-di-nucleotides (e.g. US20150056224), combinations of lecithin and carbomer homopolymers (e.g. U.S. Pat. No. 6,676,958), and saponins, such as Quil A and QS21 (see e.g. Zhu D and W Tuo, 2016, Nat Prod Chem Res 3: e113 (doi:10.4172/2329-6836.1000e113), optionally in combination with QS7 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). In certain embodiments, the adjuvant comprises Quil-A, such as for instance commercially obtainable from Brenntag (now Croda) or Invivogen. QuilA contains the water-extractable fraction of saponins from the Quillaja saponaria Molina tree. These saponins belong to the group of triterpenoid saponins, that have a common triterpenoid backbone structure. Saponins are known to induce a strong adjuvant response to T-dependent as well as T-independent antigens, as well as strong cytotoxic CD8+ lymphocyte responses and potentiating the response to mucosal antigens. They can also be combined with cholesterol and phospholipids, to form immunostimulatory complexes (ISCOMs), wherein QuilA adjuvant can activate both antibody-mediated and cell-mediated immune responses to a broad range of antigens from different origins. In certain embodiments, the adjuvant is AS01, for example AS01_{B}. AS01 is an adjuvant system containing MPL (3-O-desacyl-4′-monophosphoryl lipid A), QS21 (Quillaja saponaria Molina, fraction 21), and liposomes. In certain embodiments, the AS01 is commercially available (GSK) or can be made as described in WO 96/33739, incorporated herein by reference. Certain adjuvants comprise emulsions, which are mixtures of two immiscible fluids, e.g. oil and water, one of which is suspended as small drops inside the other and are stabilized by surface-active agents. Oil-in-water emulsions have water forming the continuous phase, surrounding small droplets of oil, while water-in-oil emulsions have oil forming the continuous phase. Certain oil-in-water emulsions comprise squalene (a metabolizable oil). Certain adjuvants comprise block copolymers, which are copolymers formed when two monomers cluster together and form blocks of repeating units. An example of a water in oil emulsion comprising a block copolymer, squalene and a microparticulate stabilizer is TiterMax®, which can be commercially obtained from Sigma-Aldrich. Optionally emulsions can be combined with or comprise further immunostimulating components, such as a TLR4 agonist. Certain adjuvants are oil in water emulsions (such as squalene or peanut oil) also used in MF59 (see e.g. EP0399843, U.S. Pat. Nos. 6,299,884, 6,451,325) and AS03, optionally in combination with immune stimulants, such as monophosphoryl lipid A and/or QS21 such as in AS02 (see Stoute et al., 1997, N. Engl. J. Med. 336, 86-91). Further examples of adjuvants are liposomes containing immune stimulants such as MPL and QS21, such as in AS01E and AS01B (e.g. US 2011/0206758). Other examples of adjuvants are CpG (Bioworld Today, Nov. 15, 1998) and imidazoquinolines (such as imiquimod and R848). See, e.g., Reed G, et al., 2013, Nature Med, 19: 1597-1608. In certain preferred embodiments according to the invention, the adjuvant is a Th1 adjuvant.

In certain preferred embodiments, the adjuvant comprises saponins, preferably the water-extractable fraction of saponins obtained from Quillaja saponaria. In certain embodiments, the adjuvant comprises QS-21.

In certain preferred embodiments, the adjuvant contains atoll-like receptor 4 (TLR4) agonist. TLR4 agonists are well known in the art, see e.g. Ireton G C and SG Reed, 2013, Expert Rev Vaccines 12: 793-807. In certain preferred embodiments, the adjuvant is a TLR4 agonist comprising lipid A, or an analog or derivative thereof.

The adjuvant, preferably including a TLR4 agonist, may be formulated in various ways, e.g. in emulsions such as water-in-oil (w/o) emulsions or oil-in-water (o/w) emulsions (examples are MF59, AS03), stable (nano-)emulsions (SE), lipid suspensions, liposomes, (polymeric) nanoparticles, virosomes, alum adsorbed, aqueous formulations (AF), and the like, representing various delivery systems for immunomodulatory molecules in the adjuvant and/or for the immunogens (see e.g. Reed et al, 2013, supra; Alving C R et al, 2012, Curr Opin Immunol 24: 310-315).

The immunostimulatory TLR4 agonist may optionally be combined with other immunomodulatory components, such as saponins (e.g. QuilA, QS7, QS21, Matrix M, Iscoms, Iscomatrix, etc), aluminum salts, activators for other TLRs (e.g. imidazoquinolines, flagellin, dsRNA analogs, TLR9 agonists, such as CpG, etc), and the like (see e.g. Reed et al, 2013, supra).

As used herein, the term “lipid A” refers to the hydrophobic lipid moiety of an LPS molecule that comprises glucosamine and is linked to keto-deoxyoctulosonate in the inner core of the LPS molecule through a ketosidic bond, which anchors the LPS molecule in the outer leaflet of the outer membrane of Gram-negative bacteria. For an overview of the synthesis of LPS and lipid A structures, see, e.g., Raetz, 1993, J. Bacteriology 175:5745-5753, Raetz C R and C Whitfield, 2002, Annu Rev Biochem 71: 635-700; U.S. Pat. Nos. 5,593,969 and 5,191,072. Lipid A, as used herein includes naturally occurring lipid A, mixtures, analogs, derivatives and precursors thereof. The term includes monosaccharides, e.g., the precursor of lipid A referred to as lipid X; disaccharide lipid A; hepta-acyl lipid A; hexa-acyl lipid A; penta-acyl lipid A; tetra-acyl lipid A, e.g., tetra-acyl precursor of lipid A, referred to as lipid IVA; dephosphorylated lipid A; monophosphoryl lipid A; diphosphoryl lipid A, such as lipid A from Escherichia coli and Rhodobacter sphaeroides. Several immune activating lipid A structures contain 6 acyl chains. Four primary acyl chains attached directly to the glucosamine sugars are 3-hydroxy acyl chains usually between 10 and 16 carbons in length. Two additional acyl chains are often attached to the 3-hydroxy groups of the primary acyl chains. E. coli lipid A, as an example, typically has four C14 3-hydroxy acyl chains attached to the sugars and one C12 and one C14 attached to the 3-hydroxy groups of the primary acyl chains at the 2′ and 3′ position, respectively.

As used herein, the term “lipid A analog or derivative” refers to a molecule that resembles the structure and immunological activity of lipid A, but that does not necessarily naturally occur in nature. Lipid A analogs or derivatives can be modified to e.g. be shortened or condensed, and/or to have their glucosamine residues substituted with another amine sugar residue, e.g. galactosamine residues, to contain a 2-deoxy-2-aminogluconate in place of the glucosamine-1-phosphate at the reducing end, to bear a galacturonic acid moiety instead of a phosphate at position 4′. Lipid A analogs or derivatives can be prepared from lipid A isolated from a bacterium, e.g., by chemical derivation, or chemically synthesized, e.g. by first determining the structure of the preferred lipid A and synthesizing analogs or derivatives thereof. Lipid A analogs or derivatives are also useful as TLR4 agonist adjuvants (see, e.g. Gregg K A et al, 2017, MBio 8, eDD492-17, doi: 10.1128/mBio.00492-17).

For example, a lipid A analog or derivative can be obtained by deacylation of a wild-type lipid A molecule, e.g., by alkali treatment. Lipid A analogs or derivatives can for instance be prepared from lipid A isolated from bacteria. Such molecules could also be chemically synthesized. Another example of lipid A analogs or derivatives are lipid A molecules isolated from bacterial cells harboring mutations in, or deletions or insertions of enzymes involved in lipid A biosynthesis and/or lipid A modification.

MPL and 3D-MPL are lipid A analogs or derivatives that have been modified to attenuate lipid A toxicity. Lipid A, MPL and 3D-MPL have a sugar backbone onto which long fatty acid chains are attached, wherein the backbone contains two 6-carbon sugars in glycosidic linkage, and a phosphoryl moiety at the 4 position. Typically, five to eight long chain fatty acids (usually 12-14 carbon atoms) are attached to the sugar backbone. Due to derivation of natural sources, MPL or 3D-MPL can be present as a composite or mixture of a number of fatty acid substitution patterns, e.g. hepta-acyl, hexa-acyl, penta-acyl, etc., with varying fatty acid lengths. This is also true for some of the other lipid A analogs or derivatives described herein, however synthetic lipid A variants can also be defined and homogeneous. MPL and its manufacture are for instance described in U.S. Pat. No. 4,436,727. 3D-MPL is for instance described in U.S. Pat. No. 4,912,094B1, and differs from MPL by selective removal of the 3-hydroxymyristic acyl residue that is ester linked to the reducing-end glucosamine at position 3 (compare for instance the structure of MPL in column 1 vs 3D-MPL in column 6 of U.S. Pat. No. 4,912,094B1). In the art often 3D-MPL is used, while sometimes referred to as MPL (e.g. the first structure in Table 1 of Ireton G C and SG Reed, 2013, supra, refers to this structure as MPL®, but actually depicts the structure of 3D-MPL).

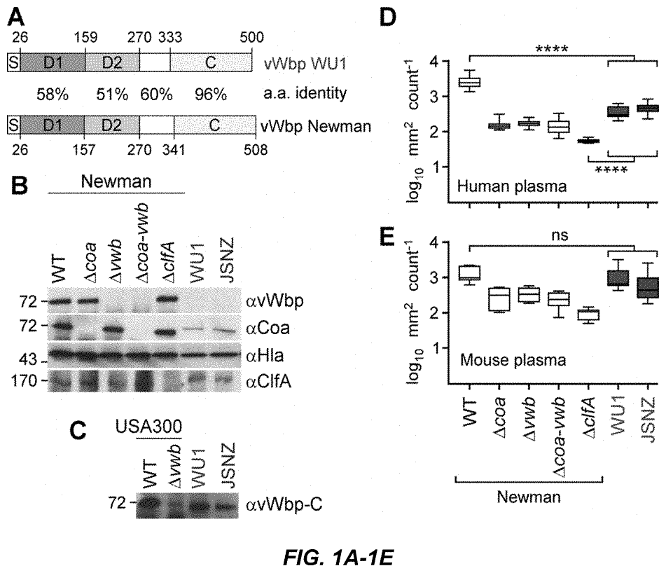

Examples of lipid A (analogs, derivatives) according to the invention include MPL, 3D-MPL, RC529 (e.g. EP1385541), PET-lipid A, GLA (glycopyranosyl lipid adjuvant, a synthetic disaccharide glycolipid; e.g. US20100310602, U.S. Pat. No. 8,722,064), SLA (e.g. Carter D et al, 2016, Clin. Transl. Immunology. 5: e108 (doi:10.1038/cti.2016.63), which describes a structure-function approach to optimize TLR4 ligands for human vaccines), PHAD (phosphorylated hexaacyl disaccharide; the structure of which is the same as that of GLA), 3D-PHAD, 3D-(6-acyl)-PHAD (3D(6A)-PHAD) (PHAD, 3D-PHAD, and 3D(6A)PHAD are synthetic lipid A variants, see e.g. avantilipids.com/divisions/adjuvants, which also provide structures of these molecules), E6020 (CAS Number 287180-63-6), ONO4007, OM-174, and the like. For exemplary chemical structures of 3D-MPL, RC529, PET-lipid A, GLA/PHAD, E6020, ONO4007, and OM-174, see e.g. Table 1 in Ireton G C and SG Reed, 2013, supra. For a structure of SLA, see e.g. FIG. 1 in Reed S G et al, 2016, Curr. Opin. Immunol. 41:85-90. In certain preferred embodiments, the TLR4 agonist adjuvant comprises a lipid A analog or derivative chosen from 3D-MPL, GLA, or SLA. In certain embodiments the lipid A analog or derivative is formulated in liposomes.

Exemplary adjuvants comprising a lipid A analog or derivative include GLA-LSQ (synthetic MPL [GLA], QS21, lipids formulated as liposomes), SLA-LSQ (synthetic MPL [SLA], QS21, lipids, formulated as liposomes), GLA-SE (synthetic MPL [GLA], squalene oil/water emulsion), SLA-SE (synthetic MPL [SLA], squalene oil/water emulsion), SLA-Nanoalum (synthetic MPL [SLA], aluminum salt), GLA-Nanoalum (synthetic MPL [GLA], aluminum salt), SLA-AF (synthetic MPL [SLA], aqueous suspension), GLA-AF (synthetic MPL [GLA], aqueous suspension), SLA-alum (synthetic MPL [SLA], aluminum salt), GLA-alum (synthetic MPL [GLA], aluminum salt), and several of the GSK ASxx series of adjuvants, including AS01 (MPL, QS21, liposomes), AS02 (MPL, QS21, oil/water emulsion), AS25 (MPL, oil/water emulsion), AS04 (MPL, aluminum salt), and AS15 (MPL, QS21, CpG, liposomes). See, e.g., WO2008/153541; WO2010/141861; WO2013/119856; WO2019/051149; WO 2013/119856; WO 2006/116423; U.S. Pat. Nos. 4,987,237; 4,436,727; 4,877,611; 4,866,034; 4,912,094; 4,987,237; 5,191,072; 5,593,969; 6,759,241; 9,017,698; 9,149,521; 9,149,522; 9,415,097; 9,415,101; 9,504,743; Reed G, et al., 2013, supra, Johnson et al., 1999, J Med Chem, 42:4640-4649, and Ulrich and Myers, 1995, Vaccine Design: The Subunit and Adjuvant Approach; Powell and Newman, Eds.; Plenum: New York, 495-524.

Non-glycolipid molecules may also be used as TLR4 agonist adjuvants, e.g. synthetic molecules such as Neoseptin-3 or natural molecules such as LeIF, see e.g. Reed S G et al, 2016, supra.

In another general aspect, the invention relates to a method of producing an immunogenic composition comprising a Staphylococcus aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide) of the invention, comprising combining a Staphylococcus aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide) with a pharmaceutically acceptable carrier to obtain the pharmaceutical composition.

Evaluation of Immunogenic Compositions

Provided herein are immunogenic compositions comprising a Staphylococcus aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide).

Various in vitro tests are used to assess the immunogenicity of the immunogenic compositions disclosed herein. For example, an in vitro opsonic assay is conducted by incubating together a mixture of staphylococcal cells, heat inactivated serum containing specific antibodies to the antigens in question, and an exogenous complement source. Opsonophagocytosis proceeds during incubation of freshly isolated polymorphonuclear cells (PMN's) or differentiated effector cells such as HL60s and the antibody/complement/staphylococcal mixture. Bacterial cells that are coated with antibody and complement are killed upon opsonophagocytosis. Colony forming units (CFU) of surviving bacteria that are recovered from opsonophagocytosis are determined by plating the assay mixture. Titers are reported as the reciprocal of the highest dilution that gives 50% bacterial killing, as determined by comparison to assay controls.

A whole cell ELISA assay is also used to assess in vitro immunogenicity and surface exposure of the antigen, wherein the bacterial strain of interest (S. aureus) is coated onto a plate, such as a 96 well plate, and the test sera from an immunized animal is reacted with the bacterial cells. If any antibody, specific for the test antigen, is reactive with a surface exposed epitope of the antigen, it can be detected by standard methods known to one skilled in the art.

Any antigen demonstrating the desired in vitro activity is then tested in an in vivo animal challenge model. In certain embodiments, immunogenic compositions are used in the immunization of an animal (e.g., a mouse) by methods and routes of immunization known to those of skill in the art (e.g., intranasal, parenteral, oral, rectal, vaginal, transdermal, intraperitoneal, intravenous, subcutaneous, etc.). Following immunization with an immunogenic composition comprising a staphylococcal antigen, the animal is challenged with a Staphylococcus sp. and assayed for resistance to staphylococcal infection.

Animal Models of Staphylococcal Infection

Several Staphylococcal Challenge Models are listed in Table 1.

TABLE 1
Staphylococcal Challenge Models

| Model | Species | Endpoint | Comments |
|---|---|---|---|
| Lethal peritonitis/ | Mouse, | live/dead | systemic model, not sensitive |
| septicemia | rabbit | | |
| Renal abscess | mouse | kidney CFU and kidney lesion | systemic model |
| Surgical wound | mouse | surgical site CFU and | local/systemic model low infecting |
| infection | | dissemination to organs | inoculum, sensitive model |
| Infective endocarditis | rat, rabbit | CFU heart value vegetation | systemic model, biofilm associated |
| Central venous catheter | rat | CFU on catheter | systemic model, biofilm associated |
| Skin and soft tissue | mouse | CFU at infection site (skin and | local infection model, biofilm |
| | | muscle) | associated |
| Air pouch infection | mouse, rat | CFU of pouch fluid | local infection model |
| Subcutaneous abscess | rabbit | CFU wiffle ball fluid | mostly local infection (abscess like |
| (wiffle ball) | | | conditions), will disseminate in |
| | | | higher inoculum environment |
| Pyelonephritis | mouse | kidney CFU | Systemic model, not reflective of |
| | | | clinical condition as a nephrotoxic |
| | | | agent is used to create kidney |
| | | | damage |
| Surgical wound | minipig | surgical site CFU and | local/systemic model. Pigs have |
| infection | | dissemination to organs | similar immune and organ systems |
| | | | as humans. Excellent translational |
| | | | model |

Murine Sepsis Model (Passive or Active)

Passive Immunization Model: Mice are passively immunized intraperitoneally (i.p.) with immune IgG or monoclonal antibody. The mice are subsequently challenged 24 hours later with a lethal dose of S. aureus. The bacterial challenge is administered intravenously (i.v.) or i.p. ensuring that any survival could be attributed to the specific in vivo interaction of the antibody with the bacteria. The bacterial challenge dose is determined to be the dose required to achieve lethal sepsis of approximately 20% of the unimmunized control mice. Statistical evaluation of survival studies can be carried out by Kaplan-Meier analysis.

Active Immunization Model: In this model, mice (e.g., Swiss Webster mice) are actively immunized intraperitoneally (i.p.) or subcutaneously (s.c.) with a target antigen at 0, 3, and 6 weeks (or other similar appropriately spaced vaccination schedule) and subsequently challenged with S. aureus at week 8 by the intravenous route. The bacterial challenge dose is calibrated to achieve approximately 20% survival in the control group over a 10-14 day period. Statistical evaluation of survival studies can be carried out by Kaplan-Meier analysis.

Infectious Endocarditis Model (Passive or Active)

A passive immunization model for infectious endocarditis (IE) caused by S. aureus has previously been used to show that ClfA can induce protective immunity (Vemachio et al., Antimicro. Agents & Chemo 50:511-8 (2006)). In this model, rabbits or rats are used to simulate clinical infections that include a central venous catheter, bacteremia, and hematogenous seeding to distal organs. Catheterized rabbits or rats with sterile aortic valve vegetations are administered a single or multiple intravenous injection of a monoclonal polyclonal antibody specific for the target antigen. Subsequently, the animals are challenged i.v. with a S. aureus or S. epidermidis strain. After challenge, heart, cardiac vegetations, and additional tissues (e.g., kidneys), and blood harvested and cultures. The frequency of staphylococcal infection in cardiac tissue, kidneys, and blood is then measured.

The infectious endocarditis model has also been adapted for active immunization studies in both rabbits and rats. Rabbits or rats are immunized intramuscularly or subcutaneously with target antigen and challenged with S. aureus two weeks later via the intravenous route.

Pyelonephritis Model

In the pyelonephritis model, mice are immunized on weeks 0, 3, and 6 (or other appropriately spaced immunization schedule) with the target antigens. Subsequently, the animals are challenged i.p. or i.v. with S. aureus PFESA0266. After 48 hours, the kidneys are harvested and bacterial CFU are counted.

Renal Abscess Model

Mice are immunized (active, 3-times with 2-weeks between doses; passive, 24 hours prior to infection, i.p.) then infected systemically infected (i.v. or r.o. (retro-orbitally)) with S. aureus. Four to seven days post-infection the mice are euthanized, kidneys scored using a semi qualitative scoring system to evaluate number of lesions in addition to approx. percentage of kidney damage (discoloration). Kidneys are then evaluated for bacterial burden.

Surgical Wound Infection Model

Mice are immunized (active, 3-times with 2-weeks between doses; passive, 24 hours prior to infection, i.p.). Two weeks after the last dose of vaccine, animals are anesthetized, thigh shaved and disinfected. An incision is made in the skin and muscle layers (to the depth of the femur). 5 ul of S. aureus is pipetted into the wound, then the muscle is closed with 4-0 silk suture and the skin is closed with autoclips. Three days later, mice are euthanized, surgical site muscle removed and enumerated for bacterial burden.

Minipig Deep Surgical Wound Infection Model

Pigs are thought to be an excellent translational model for vaccines in human clinical bacterial diseases (Gerdts et al., ILAR Journal 56 (1): 53-62 (2015)). Pigs have been used to study cystic fibrosis (Meyerholz, Theriogenology 86 (1):427-432 (2016)), sexually transmitted diseases (Kaser et al., Infection, Genetics and Evolution (2017)), pertussis (Elahi et al., Trends in Microbiology 15 (10) (2007)), osteomyelitis (Jensen et al., In Vivo 29: 555-560 (2015)), Elvang et al., In Vivo 24: 257-264 (2010)), and skin infections (Klein et al., Biofouling 34 (2): 226-236 (2018)). The pig immune system is >80% similar to humans as compared to <10% for mice (Dawson et al., BMC Genomics 14:332 (2013)). A high percentage of circulating neutrophils, similar toll-like receptors and dendritic cells are some of the immune system attributes that both pigs and humans have in common (Meurens et al., Trends in Microbiology 20 (1): 50-57 (2012)). Additionally, pig share similarities with human organ systems, i.e., skin and skin structure (Summerfield et al., Mol Immunol 66: 1-21 (2015)). These similarities make the pig an excellent model to study and translate staphylococcal diseases to human.

Methods of Use

In another general aspect, the invention relates to a method of inducing an immune response in a subject in need thereof. The methods comprise administering to the subject in need thereof an immunogenic composition of the invention.

In another general aspect, the invention relates to a method of treating or preventing a Staphylococcus infection in a subject in need thereof. The methods comprise administering to the subject in need thereof an immunogenic composition of the invention.

According to embodiments of the invention, the immunogenic composition comprises a therapeutically effective amount of a Staphylococcus aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide). As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein, “a subject in need of therapeutic or preventative immunity” refers to a subject in which it is desirable to treat, i.e., to prevent, cure, slow down, or reduce the severity of Staphylococcus related symptoms over a specified period of time. As used herein, “a subject in need of an immune response” refers to a subject for which an immune response against any LukAB and/or SpA expressing Staphylococcus strain is desired.

As used herein with reference to a Staphylococcus aureus protein A (SpA) variant polypeptide and a mutant staphylococcal leukocidin subunit polypeptide (e.g., the staphylococcal LukA polypeptide, the staphylococcal LukB polypeptide, and/or the staphylococcal LukAB dimer polypeptide), a therapeutically effective amount means an amount of the Staphylococcus aureus protein A (SpA) variant polypeptide and the mutant staphylococcal leukocidin subunit polypeptide (e.g., the staphylococcal LukA polypeptide, the staphylococcal LukB polypeptide, and/or the staphylococcal LukAB dimer polypeptide) that modulates an immune response in a subject in need thereof.

In certain embodiments, the immunogenic composition further comprises an adjuvant.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a Staphylococcus infection, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a fever, chills, blisters, boils, rashes, skin redness, and abscesses. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment and/or prevention of a Staphylococcus infection in a subject in need thereof. For treatment and/or prevention of a Staphylococcus infection, the compositions can be used in combination with another treatment including, but not limited to, at least one antibiotic. The at least one antibiotic can, for example, be selected from the group consisting of streptomycin, ciprofloxacin, doxycycline, gentamycin, chloramphenicol, trimethoprim, sulfamethoxazole, ampicillin, tetracycline, and combinations thereof.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### EMBODIMENTS

The invention provides also the following non-limiting embodiments.

Embodiment 1 is an immunogenic composition comprising:
(a) a Staphylococcus aureus protein A (SpA) variant polypeptide, wherein the SpA variant polypeptide comprises at least one SpA A, B, C, D, or E domain; and
(b) a mutant staphylococcal leukocidin subunit polypeptide comprising:
   (i) a mutant LukA polypeptide,
   (ii) a mutant LukB polypeptide, and/or
   (iii) a mutant LukAB dimer polypeptide,
      wherein (i), (ii), and/or (iii) have one or more amino acid substitutions, deletions, or a combination thereof,
      such that the ability of the mutant LukA, LukB, and/or LukAB polypeptides to form pores in the surface of eukaryotic cells is disrupted, thereby reducing the toxicity of the mutant LukA and/or LukB polypeptide or the mutant LukAB dimer polypeptide relative to the corresponding wild-type LukA and/or LukB polypeptide or LukAB dimer polypeptide.

Embodiment 2 is the immunogenic composition of embodiment 1, wherein the SpA variant polypeptide has at least one amino acid substitution that disrupts Fc binding and at least a second amino acid substitution that disrupts V_{H}3 binding.

Embodiment 3 is the immunogenic composition of embodiment 1 or 2, wherein the SpA variant polypeptide comprises a SpA D domain and has an amino acid sequence that has at least 90%, at least 91%, at least 92%, at least 93%, at least 94%, at least 95%, at least 96%, at least 97%, at least 98%, or at least 99% identity to the amino acid sequence of SEQ ID NO:58.

Embodiment 4 is the immunogenic composition of embodiment 3, wherein the SpA variant polypeptide has one or more amino acid substitutions at amino acid position 9 or 10 of SEQ ID NO:58.

Embodiment 5 is the immunogenic composition of embodiment 3 or 4, wherein the SpA variant polypeptide further comprises a SpA E, A, B, or C domain.

Embodiment 6 is the immunogenic composition of embodiment 5, wherein the SpA variant polypeptide comprises a SpA E, A, B, and C domain and has an amino acid sequence that has at least 80%, at least 90%, at least 91%, at least 92%, at least 93%, at least 94%, at least 95%, at least 96%, at least 97%, at least 98%, or at least 99% identity to the amino acid sequence of SEQ ID NO:54.

Embodiment 7 is the immunogenic composition of embodiment 5 or 6, wherein each SpA E, A, B, and C domain has one or more amino acid substitutions at positions corresponding to amino acid positions 9 and 10 of SEQ ID NO:58.

Embodiment 8 is the immunogenic composition of any one of embodiments 4 to 7, wherein the amino acid substitution is a lysine residue for a glutamine residue.

Embodiment 9 is the immunogenic composition of any one of embodiments 5 to 8, wherein each SpA D, E, A, B, and C domain has one or more amino acid substitutions at positions corresponding to amino acid positions 36 and 37 of SEQ ID NO:58.

Embodiment 10 is the immunogenic composition of embodiment 1, wherein the SpA variant polypeptide comprises an amino acid sequence selected from SEQ ID NO:72, SEQ ID NO:77, SEQ ID NO:82, or SEQ ID NO:88.

Embodiment 11 is the immunogenic composition of any one of embodiments 1 to 4, wherein said SpA variant polypeptide comprises at least one SpA A, B, C, D, or E domain, and wherein the at least one domain has (i) lysine substitutions for glutamine residues corresponding to positions 9 and 10 in the SpA D domain (SEQ ID NO:58) and (ii) a glutamate substitution corresponding to position 33 in the SpA D domain (SEQ ID NO:58).

Embodiment 12 is the immunogenic composition of embodiment 11, wherein the SpA variant polypeptide does not, relative to a negative control, detectably crosslink IgG and IgE in blood or activate basophils.

Embodiment 13 is the immunogenic composition of embodiment 11 or 12, wherein the SpA variant polypeptide has a reduced K_{A} binding affinity for V_{H}3 from human IgG as compared to a SpA variant polypeptide (SpA_{KKAA}) that comprises lysine substitutions for glutamine residues in each SpA A, B, C, D, and E domain corresponding to positions 9 and 10 in the SpA D domain (SEQ ID NO:58) and alanine substitutions for aspartic acid residues in each SpA A, B, C, D, and E domain corresponding to positions 36 and 37 of the SpA D domain (SEQ ID NO:58).

Embodiment 14 is the immunogenic composition of any one of embodiments 1 to 13, wherein the SpA variant polypeptide has a K_{A} binding affinity for V_{H}3 from human IgG that is reduced by at least 2-fold as compared to SpA_{KKAA}.

Embodiment 15 is the immunogenic composition of any one of embodiments 1 to 14, wherein the SpA variant polypeptide has a K_{A} binding affinity for V_{H}3 from human IgG of less than 1×10⁵ M⁻¹.

Embodiment 16 is the immunogenic composition of any one of embodiments 1 to 15, wherein the SpA variant polypeptide does not have substitutions in any of the SpA A, B, C, D, or E domains corresponding to amino acid positions 36 and 37 in the SpA D domain.

Embodiment 17 is the immunogenic composition of any one of embodiments 11 to 16, wherein the only substitutions in the SpA variant polypeptide are (i) and (ii).

Embodiment 18 is an immunogenic composition comprising:
(a) a Staphylococcus aureus protein A (SpA) variant polypeptide, wherein said SpA variant polypeptide comprises at least one SpA A, B, C, D, or E domain, and wherein said domain has (i) lysine substitutions for glutamine residues in the at least one SpA A, B, C, D, or E domain corresponding to positions 9 and 10 in the SpA D domain (SEQ ID NO:58) and (ii) a threonine substitution in the at least one SpA A, B, C, D, or E domain corresponding to position 33 in the SpA D domain (SEQ ID NO:58), wherein the polypeptide does not, relative to a negative control, detectably crosslink IgG and IgE in blood or activate basophils; and
(b) a mutant staphylococcal leukocidin subunit polypeptide comprising:
   (1) a mutant LukA polypeptide,
   (2) a mutant Luk B polypeptide, and/or
   (3) a mutant LukAB dimer polypeptide,
      wherein (1), (2), and/or (3) have one or more amino acid substitutions, deletions, or a combination thereof.
      such that the ability of the mutant LukA, LukB, and/or LukAB polypeptides to form pores in the surface of eukaryotic cells is disrupted, thereby reducing the toxicity of the mutant LukA and/or LukB polypeptide or the mutant LukAB dimer polypeptide relative to the corresponding wild-type LukA and/or LukB polypeptide or LukAB dimer polypeptide.

Embodiment 19 is the immunogenic composition of embodiment 18 wherein the SpA variant polypeptide has a reduced K_{A} binding affinity for V_{H}3 from human IgG as compared to a SpA variant polypeptide (SpA_{KKAA}) comprising lysine substitutions for glutamine residues in each SpA A-E domain corresponding to positions 9 and 10 in the SpA D domain (SEQ ID NO:58) and alanine substitutions for aspartic acid residues in each SpA-E domain corresponding to positions 36 and 37 of the SpA D domain (SEQ ID NO:58).

Embodiment 20 is the immunogenic composition of embodiment 18 or 19 wherein the SpA variant polypeptide has a K_{A} binding affinity for V_{H}3 from human IgG that is reduced by at least 2-fold as compared to SpA_{KKAA}.

Embodiment 21 is the immunogenic composition of any one of embodiments 18 to 20, wherein the SpA variant polypeptide has a K_{A} binding affinity for V_{H}3 from human IgG is less than 1×10⁵ M⁻¹.

Embodiment 22 is the immunogenic composition of any one of embodiments 18 to 21, wherein the SpA variant polypeptide does not have substitutions in any of the SpA A, B, C, D, or E domains corresponding to amino acid positions 36 and 37 in the SpA D domain.

Embodiment 23 is the immunogenic composition of any one of embodiments 18 to 22, wherein the only substitutions in the SpA variant polypeptide are (i) and (ii).

Embodiment 24 is the immunogenic composition of any one of embodiments 1 to 5 or 18 to 22, wherein the SpA variant polypeptide comprises SEQ ID NO:66 or SEQ ID NO:71.

Embodiment 25 is the immunogenic composition of any one of embodiments 1 to 5 or 18 to 22, wherein the SpA variant polypeptide comprises SEQ ID NO:66.

Embodiment 26 is the immunogenic composition of any one of embodiments 1 to 5 or 18 to 22, wherein the SpA variant polypeptide comprises SEQ ID NO:60.

Embodiment 27 is the immunogenic composition of any one of embodiments 18 to 23, wherein the immunogenic composition comprises SEQ ID NO:61.

Embodiment 28 is an immunogenic composition comprising:
(a) a Staphylococcus aureus protein A (SpA) variant polypeptide, wherein the SpA variant polypeptide comprises at least one SpA A, B, C, D, and E domain, and wherein said domain has (i) lysine substitutions for glutamine residues corresponding to positions 9 and 10 of the SpA D domain (SEQ ID NO:58) in each of domains A, B, C, D, and E, and (ii) at least one other amino acid substitution corresponding to position 29 and/or 33 of the SpA D domain (SEQ ID NO:58) in each of domains A, B, C, D, and E, wherein the SpA variant has a K_{D} binding affinity for VH3 from human IgG that is greater than 1.0×10⁻⁴ M and/or a K_{D} binding affinity for VH3 from human IgE that is greater than 1.0×10⁻⁶ M; and
(b) a mutant staphylococcal leukocidin subunit polypeptide comprising:
   (1) a mutant LukA polypeptide,
   (2) a mutant Luk B polypeptide, and/or
   (3) a mutant LukAB dimer polypeptide,
      wherein (1), (2), and/or (3) have one or more amino acid substitutions, deletions, or a combination thereof.
      such that the ability of the mutant LukA, LukB, and/or LukAB polypeptides to form pores in the surface of eukaryotic cells is disrupted, thereby reducing the toxicity of the mutant LukA and/or LukB polypeptide or the mutant LukAB dimer polypeptide relative to the corresponding wild-type LukA and/or LukB polypeptide or LukAB dimer polypeptide.

Embodiment 29 is the immunogenic composition of embodiment 28, wherein the SpA variant polypeptide comprises an amino acid substitution corresponding to position 29 of the SpA D domain (SEQ ID NO:58) in each of domains A, B, C, D, and E.

Embodiment 30 is the immunogenic composition of embodiment 28, wherein the SpA variant polypeptide comprises an amino acid substitution corresponding to position 33 of the SpA D domain (SEQ ID NO:58) in each of domains A, B, C, D, and E.

Embodiment 31 is the immunogenic composition of embodiment 29, wherein the SpA variant polypeptide comprises an amino acid substitution corresponding to positions 29 and 33 of the SpA D domain (SEQ ID NO:58) in each of domains A, B, C, D, and E.

Embodiment 32 is the immunogenic composition of any one of embodiments 28 to 31, wherein the SpA variant polypeptide comprises an amino acid substitution corresponding to one or both positions 36 and 37 of the SpA D domain (SEQ ID NO:58) in each of domains A, B, C, D, and E.

Embodiment 33 is the immunogenic composition of embodiment 32, wherein the SpA variant polypeptide comprises an amino acid substitution corresponding to both positions 36 and 37 of the SpA D domain (SEQ ID NO:58) in each of domains A, B, C, D, and E.

Embodiment 34 is the immunogenic composition of embodiments 32 or 33, wherein the amino acid substitutions corresponding to positions 36 and 37 of the SpA D domain (SEQ ID NO:58) are alanine residues for aspartic acid residues.

Embodiment 35 is the immunogenic composition of any one of embodiments 28 to 34, wherein the SpA variant polypeptide comprises variant A, B, C, D, and E domains that are at least 70% identical to the amino acid sequence of SEQ ID NO:58.

Embodiment 36 is the immunogenic composition of embodiment 35, wherein the SpA variant polypeptide comprises variant A, B, C, D, and E domains that are at least 80% identical to the amino acid sequence of SEQ ID NO:58.

Embodiment 37 is the immunogenic composition of embodiment 36, wherein the SpA variant polypeptide comprises variant A, B, C, D, and E domains that are at least 90% identical to the amino acid sequence of SEQ ID NO:58.

Embodiment 38 is the immunogenic composition of embodiment 37, wherein the SpA variant polypeptide comprises variant A, B, C, D, and E domains that do not comprise any amino acid substitutions in SEQ ID NO:58 except at the corresponding positions 9, 10, 29, 33, 36, and/or 37.

Embodiment 39 is the immunogenic composition of embodiment 38, wherein the SpA variant polypeptide comprises variant A, B, C, D, and E domains consisting only of amino acid substitutions corresponding to positions 9, 10, and 29 of SEQ ID NO:58.

Embodiment 40 is the immunogenic composition of embodiment 38, wherein the SpA variant polypeptide comprises variant A, B, C, D, and E domains consisting only of amino acid substitutions corresponding to positions 9, 10, and 33 of SEQ ID NO:58.

Embodiment 41 is the immunogenic composition of embodiment 38, wherein the SpA variant polypeptide comprises variant A, B, C, D, and E domains consisting only of amino acid substitutions corresponding to positions 9, 10, 29, and 33 of SEQ ID NO:58.

Embodiment 42 is the immunogenic composition of embodiment 38, wherein the SpA variant polypeptide comprises variant A, B, C, D, and E domains consisting only of amino acid substitutions corresponding to positions 9, 10, 29, 36, and 37 of SEQ ID NO:58.

Embodiment 43 is the immunogenic composition of embodiment 38, wherein the SpA variant polypeptide comprises variant A, B, C, D, and E domains consisting only of amino acid substitutions corresponding to positions 9, 10, 33, 36, and 37 of SEQ ID NO:58.

Embodiment 44 is the immunogenic composition of embodiment 38, wherein the SpA variant polypeptide comprises variant A, B, C, D, and E domains consisting only of amino acid substitutions corresponding to positions 9, 10, 29, 33, 36, and 37 of SEQ ID NO:58.

Embodiment 45 is the immunogenic composition of any one of embodiments 29 to 44, wherein the substitution of the amino acid corresponding to position 29 of SEQ ID NO:58 is alanine, leucine, proline, phenylalanine, glutamic acid, arginine, lysine, serine, threonine, or glutamine.

Embodiment 46 is the immunogenic composition of embodiments 45, wherein the substitution of the amino acid corresponding to position 29 of SEQ ID NO:58 is alanine, phenylalanine, or arginine.

Embodiment 47 is the immunogenic composition of any one of embodiments 30 to 44, wherein the substitution of the amino acid corresponding to position 33 of SEQ ID NO:58 is alanine, phenylalanine, glutamic acid, lysine, or glutamine.

Embodiment 48 is the immunogenic composition of any one of embodiments 29 to 44, wherein the substitution of the amino acid corresponding to position 33 of SEQ ID NO:58 is phenylalanine, glutamic acid, or glutamine.

Embodiment 49 is the immunogenic composition of any one of embodiments 28 to 48, wherein the SpA variant polypeptide has a K_{D} binding affinity for VH3 that is greater than 1.0×10-M.

Embodiment 50 is the immunogenic composition of embodiment 49, wherein the SpA variant polypeptide comprises the amino acid sequence of SEQ ID NO:60 or SEQ ID NO:61.

Embodiment 51 is the immunogenic composition of embodiment 1, 18, or 28, wherein the SpA variant polypeptide comprises the amino acid sequence of any one of SEQ ID NOs:72-88.

Embodiment 52 is the immunogenic composition of any one of embodiments 1 to 51, wherein the mutant LukA polypeptide comprises an amino acid sequence having at least 80%, at least 90%, at least 91%, at least 92%, at least 93%, at least 94%, at least 95%, at least 96%, at least 97%, at least 98%, or at least 99% sequence identity to any one of SEQ ID NOs:1-28.

Embodiment 53 is the immunogenic composition of embodiment 52, wherein the mutant LukA polypeptide comprises a deletion of the amino acid residues corresponding to amino acid positions 342-351 of any one of SEQ ID NOs:1-14 and at amino acid positions 315-324 of any one of SEQ ID NOs:15-28.

Embodiment 54 is the immunogenic composition of any one of embodiments 1 to 53, wherein the mutant LukB polypeptide comprises an amino acid sequence having at least 80%, at least 90%, at least 91%, at least 92%, at least 93%, at least 94%, at least 95%, at least 96%, at least 97%, at least 98%, or at least 99% sequence identity to any of SEQ ID NO:29-53.

Embodiment 55 is the immunogenic composition of any one of embodiments 1 to 54, wherein the mutant LukAB dimer polypeptide comprises a mutant LukA polypeptide comprising an amino acid sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs:1-28; and a mutant LukB polypeptide comprising an amino acid sequence having at least 80%, at least 85%, at least 90%, at least 91%, at least 92%, at least 93%, at least 94%, at least 95%, at least 96%, at least 97%, at least 98%, or at least 99% identity to any of SEQ ID NO:29-53.

Embodiment 56 is the immunogenic composition of any one of embodiments 1 to 55, wherein the mutant LukAB dimer polypeptide comprises a mutant LukA polypeptide comprising an amino acid sequence having at least 80%, at least 85%, at least 90%, at least 91%, at least 92%, at least 93%, at least 94%, at least 95%, at least 96%, at least 97%, at least 98%, or at least 99% identity to a LukA polypeptide having a deletion of the amino acid residues corresponding to positions 315-324 of SEQ ID NO: 16; and a mutant LukB polypeptide comprising an amino acid sequence having at least 80%, at least 85%, at least 90%, at least 91%, at least 92%, at least 93%, at least 94%, at least 95%, at least 96%, at least 97%, at least 98%, or at least 99% identity to the amino acid sequence of SEQ ID NO:53.

Embodiment 57 is the immunogenic composition of any one of embodiments 1 to 56, wherein the mutant LukAB dimer polypeptide comprises a mutant LukA polypeptide having a deletion of the amino acid residues corresponding to positions 315-324 of SEQ ID NO:16; and a mutant LukB polypeptide comprising the amino acid sequence of SEQ ID NO: 53.

Embodiment 58 is the immunogenic composition of any one of embodiments 1 to 57, wherein the mutant LukAB dimer polypeptide comprises a mutant LukA polypeptide with a D39A amino acid substitution corresponding to SEQ ID NO:15 and a LukB polypeptide with an R23E amino acid substitution corresponding to SEQ ID NO: 42.

Embodiment 59 is the immunogenic composition of any one of embodiments 1 to 58, further comprising an adjuvant.

Embodiment 60 is the immunogenic composition of embodiment 59, wherein the adjuvant comprises saponins.

Embodiment 61 is the immunogenic composition of embodiment 60, wherein the saponin is QS21.

Embodiment 62 is the immunogenic composition of embodiment 61, wherein the adjuvant comprises a TLR4 agonist.

Embodiment 63 is the immunogenic composition of embodiment 62, wherein the TLR4 agonist is lipid A or an analog or derivative thereof.

Embodiment 64 is the immunogenic composition of embodiment 63, wherein the TLR4 agonist comprises MPL, 3D-MPL, RC529, GLA, SLA, E6020, PET-lipid A, PHAD, 3D-PHAD, 3D-(6-acyl)-PHAD, ONO4007, or OM-174.

Embodiment 65 is the immunogenic composition of embodiment 62, wherein the TLR4 agonist comprises GLA.

Embodiment 66 is the immunogenic composition of embodiment 59, wherein the adjuvant comprises MPL, QS21, and liposomes.

Embodiment 67 is the immunogenic composition of embodiment 59 or 62, wherein the adjuvant is formulated in an oil in water emulsion, such as MF59 or AS03.

Embodiment 68 is the immunogenic composition of embodiment 59, 62, or 65, wherein the adjuvant is formulated in an oil in water emulsion comprising squalene.

Embodiment 69 is the immunogenic composition of embodiment 65, wherein the adjuvant further comprises QS21 and liposomes.

Embodiment 70 is the immunogenic composition of embodiment 59, wherein the adjuvant comprises GLA-SE.

Embodiment 71 is the immunogenic composition of embodiment 59, wherein the adjuvant comprises GLA-SLQ.

Embodiment 72 is the immunogenic composition of any one of embodiments 1 to 71, further comprising at least one staphylococcal antigen or immunogenic fragment thereof selected from the group consisting of CP5, CP8, Eap, Ebh, Emp, EsaB, EsaC, EsxA, EsxB, EsxAB(fusion), SdrC, SdrD, SdrE, IsdA, IsdB, IsdC, ClfA, ClfB, Coa, Hla, mHla, MntC, rTSST-1, rTSST-1v, TSST-1, SasF, vWbp, vWh vitronectin binding protein, Aaa, Aap, Ant, autolysin glucosaminidase, autolysin amidase, Can, collagen binding protein, CsalA, EFB, Elastin binding protein, EPB, FbpA, fibrinogen binding protein, Fibronectin binding protein, FhuD, FhuD2, FnbA, FnbB, GehD, HarA, HBP, Immunodominant ABC transporter, IsaA/PisA, laminin receptor, Lipase GehD, MAP, Mg2+ transporter, MHC II analog, MRPII, NPase, RNA III activating protein (RAP), SasA, SasB, SasC, SasD, SasK, SBI, SdrF, SdrG, SdrH, SEA exotoxins, SEB exotoxins, mSEB, SitC, Ni ABC transporter, SitC/MntC/saliva binding protein, SsaA, SSP-1, SSP-2, Spa5, SpAKKAA, SpAkR, Sta006, Sta011, PVL, LukED and Hlg.

Embodiment 73 is the immunogenic composition of any one of embodiments 1 to 72, further comprising an Hla, staphylococcal antigen.

Embodiment 74 is one or more isolated nucleic acids encoding a Staphylococcus aureus protein A (SpA) variant polypeptide and a mutant Luk A polypeptide, a mutant Luk B polypeptide, or a mutant LukAB dimer polypeptide according to any one of embodiments 1 to 73.

Embodiment 75 is a vector comprising the isolated nucleic acid of embodiment 74.

Embodiment 76 is an isolated host cell comprising the vector of embodiment 75.

Embodiment 77 is a method for treating or preventing a Staphylococcus infection in a subject in need thereof, the method comprising administering to the subject in need thereof an effective amount of the immunogenic composition of any one of embodiments 1 to 73, one or more isolated nucleic acids of embodiment 74, a vector of embodiment 75, or a host cell of embodiment 76.

Embodiment 78 is a method for eliciting an immune response to a Staphylococcus bacterium in a subject in need thereof, the method comprising administering to the subject in need thereof an effective amount of the immunogenic composition of any one of embodiments 1 to 73, one or more isolated nucleic acids of embodiment 74, a vector of embodiment 75, or a host cell of embodiment 76.

Embodiment 79 is a method for decolonization or preventing colonization or recolonization of a Staphylococcus bacterium in a subject in need thereof, the method comprising administering to the subject in need thereof an effective amount of the immunogenic composition of any one of embodiments 1 to 73, one or more isolated nucleic acids of embodiment 74, a vector of embodiment 75, or a host cell of embodiment 76.

### EXAMPLES

### Example 1 Staphylococcal Protein a Contributes to Persistent Colonization of Mice with Staphylococcus aureus

Staphylococcus aureus persistently colonizes the nasopharynx of about a third of the human population, thereby promoting community- and hospital-acquired infections. Antibiotics are currently used for decolonization of individuals at increased risk of infection. However, the efficacy of antibiotics is limited by recolonization and selection for drug-resistant strains. Nasal colonization triggers IgG responses against staphylococcal surface antigens, however these antibodies cannot prevent subsequent colonization or disease. This example describes S. aureus WU1, a multi-locus sequence type ST88 isolate, that persistently colonizes the nasopharynx of mice. It is reported here that staphylococcal protein A (SpA) is required for persistence of S. aureus WU1 in the nasopharynx. Compared to animals colonized by wild-type S. aureus, mice colonized with the Δspa variant mount increased IgG responses against staphylococcal colonization determinants. Immunization of mice with a non-toxigenic SpA variant, which cannot crosslink B cell receptors and divert antibody responses, elicits protein A-neutralizing antibodies that promote IgG responses against colonizing S. aureus and diminish pathogen persistence.

Results

Staphylococcus aureus WU1.

An outbreak of preputial gland infections of male C57BL/6 mice was observed in an animal breeding colony. Samples were collected from preputial gland adenitis (PGA) and from the nasopharynx of male and female C75BL/6J mice and analyzed by growth on mannitol-salt agar (MSA) and Baird-Parker agar (BPA). Multi-locus sequence typing and spa genotyping revealed that animals had been colonized with S. aureus ST88 spa genotype t186, which was also responsible for PGA in male mice. S. aureus CC88 with spa genotype t186 have been reported before as stably colonizing isolates from laboratory mice in the United States (37). Other spa genotypes include t325, t448, t690, t755, t786, t2085, t2815, t5562, t11285 and t12341 (37). The New Zealand JSNZ isolate carries the distinct spa genotype t729 (37). Nonetheless, both S. aureus JSNZ and WU1 share the type 8 capsular polysaccharide genes and lack the mecA gene as well as mobile-genetic element (MGE) encoded T cell superantigens (37). Further, the hlb-converting phage that expresses human-specific immune evasion cluster 1 (IEC1) genes sak (staphylokinase), chp (CHIPS, chemotaxis inhibitory protein of S. aureus) and scn (SCIN-A, staphylococcal complement inhibitor A) is absent in the genome of WU1 resulting in an intact α-hemolysin encoding gene (hlb)(38). Of note, the WU1 encoded IEC2 carries the scn homologue scb/scc (SCIN-B/-C) along with hla (α-hemolysin) and ssl12-14 (staphylococcal superantigen-like 12-14) (39). Unlike other CC88 isolates that stably colonize mice (37), the genome of WU1 harbors the blaZ gene. When analyzed for genes encoding sortase-anchored surface proteins, it was observed that S. aureus WU1 carries genes for determinants previously associated with nasal colonization, including ClfB, IsdA, SdrC, SdrD, and SasG (TABLE 2).

TABLE 2
Conservation of protein products of select open reading frames
in the genomes of S. aureus WU1, JSNZ and Newman

Amino acid identity (%) WU1 gene product

| Protein | JSNZ | Newman |
|---|---|---|
| SpA | 99 | 98 |
| ClfA | 100 | 93 |
| ClfB | 100 | 96 |
| FnBPA | 100 | 82 |
| FnBPB | 87 | 87 |
| IsdA | 100 | 100 |
| IsdB | 99 | 98 |
| SdrC | 100 | 100 |
| SdrD | 95 | 95 |
| SdrE | 100 | 98 |
| EsxA | 100 | 100 |
| EsxB | 100 | 100 |
| SasA | 100 | 99 |
| SasD | 100 | 99 |
| SasF | 100 | 98 |
| SasI | 99 | 100 |
| SasG | 100 | 69 |
| SasK | 100 | 93 ^{a} |
| Coa | 98 | 98 |
| vWbp | 100 | 71 |
| Hla | 78 | 99 |
| SCIN | 100 | 45 ^{b} |
| Eap | 100 | 99 |
| Efb | 100 | 99 |
| Ebh | 99 | 98 |
| TarS | 100 | 98 |

^{a} Compared to the S. aureus 04-02981 strain
^{b} Compared to the S. aureus USA300 strain

S. aureus abscess formation has been linked to determinants of bacterial agglutination with fibrin (40, 41). Agglutination requires two S. aureus secreted products that activate host prothrombin to convert fibrinogen into fibrin:coagulase (Coa) and von Willebrand factor binding protein (vWbp) (40). Clumping factor A (ClfA) binds fibrinogen and coats staphylococci with coagulase-generated fibrin fibrils, thereby interfering with S. aureus uptake and killing by host phagocytes (41, 42). The clfA gene is identical in S. aureus WU1 and JSNZ yet displays allele-specific differences with clfA from S. aureus Newman (TABLE 2), a CC8 human clinical isolate that is used routinely for laboratory challenge experiments with mice (43). The observed differences in clfA are however clade specific, as they can be found in CC88 strains isolated either from human or from murine hosts (data not shown). The coa gene products of S. aureus WU1, JSNZ and Newman are virtually identical (TABLE 2). In contrast, the product of the vwb gene of S. aureus WU1 and JSNZ differs significantly from S. aureus Newman with the greatest sequence variation in the prothrombin-binding D1 and D2 domains (FIG. 1A) and were not recognized by polyclonal antibodies raised against Newman vWbp (FIG. 1B). Secreted vWbp from the two CC88 strains could be recognized by a serum that had been raised against the conserved C-terminal domain of vWbp from strain USA300 (FIG. 1C). In contrast to S. aureus Newman, which secretes large amounts of Coa and rapidly agglutinates human and mouse plasma, S. aureus WU1 and JSNZ secrete less Coa and agglutinate mouse plasma more readily than human plasma as compared to strain Newman (FIG. 1B, 1D, 1E). The coagulase activity of S. aureus Newman is dependent on coa and vwb expression, as the corresponding Δcoa, A vwb and Δcoa Δvwb mutants displayed agglutination defects in mouse and human plasma (FIG. 1D, 1E). Taken together, these data suggest that the ST88 allele of the vwb gene in S. aureus WU1 and JSNZ may promote efficient prothrombin-mediated coagulation and fibrin agglutination in mouse plasma, which may support the pathogenesis of invasive diseases such as PGA.

S. aureus WU1 Persistently Colonizes the Nasopharynx of Mice.

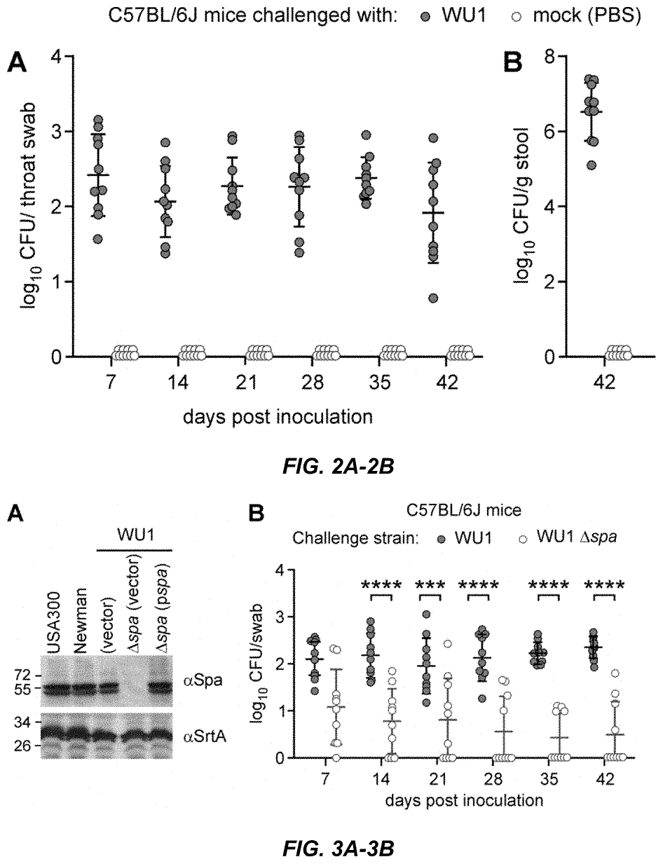

To analyze S. aureus WU1 for its ability to colonize mice, cohorts (n=10) of female C57BL/6 animals were analyzed by spreading pharyngeal swabs and fecal material on BPA. Naïve mice lacking bacterial growth on BPA were anesthetized and inoculated by pipetting 10 μl suspensions of 1×10⁸ CFU S. aureus WU1 in phosphate-buffered saline (PBS) into the right nostril. Animals were analyzed for colonization by swabbing the oropharynx in weekly intervals, i.e. 7, 14, 21, 28, 35, and 42 days following inoculation. Swabs were spread on BPA, incubated for colony formation and enumerated (FIG. 2A). Even without prior antibiotic treatment or antibiotic selection, S. aureus WU1 colonized experimental animals with a load ranging from 1.2-2.9 log₁₀ CFU per swab over 42 days (FIG. 2A). To validate persistent colonization with S. aureus WU1, colonies obtained after 42 days were analyzed by MLST and spa genotyping. The data showed that mice were still colonized with ST88 spa t186, indicating that S. aureus WU1 persistently colonizes the nasopharynx of C57BL/6 mice. As a control, mock PBS inoculation of cohorts of C57BL/6J animals in separate cages that were maintained in the same animal facility room and the same cage racks as S. aureus WU1 colonized animals did not lead to staphylococcal colonization of the nasopharynx (FIG. 2A). Day 42 stool samples from mice were homogenized in PBS and plated on mannitol salt agar (MSA) for CFU enumeration (FIG. 2B). Stool samples of S. aureus WU1 colonized mice harbored 5.1-7.3 log 10 CFU g-1 feces, indicating that the gastrointestinal (GI) tract was also colonized with the S. aureus WU1 strain. As a control, mock (PBS) inoculated mice did not harbor S. aureus in their stool samples (FIG. 2B).

S. aureus WU1 colonization triggers serum IgG response in mice. Earlier work generated the S. aureus antigen matrix, which is comprised of 25 conserved secreted proteins. Each of the 25 recombinant affinity-tagged proteins was purified and immobilized on membrane filter (44). To measure host immune responses during colonization, naïve or S. aureus WU1 colonized animals were bled 15 days after inoculation and serum IgG responses were analyzed by incubation with the S. aureus antigen matrix. IgG binding was detected with IRDye 680-conjugated goat anti-mouse IgG (LI-COR) and quantified by infrared imaging. This experiment demonstrated that S. aureus WUT colonization led to increases in serum IgG directed against the sortase-anchored surface proteins ClfA, ClfB, IsdA, and IsdB and to the giant extracellular matrix bind protein (Ebh), a cell size and peptidoglycan synthesis determinant of S. aureus (45) (TABLE 3).

TABLE 3
Serum IgG responses in C57BL/6J mice colonized with S. aureus WU1 or its Δspa variant

| | | | | WU1Δspa | | WU1Δspa | |
|---|---|---|---|---|---|---|---|
| | | WU1 | | (colonized) | | (cleared) | |
| | | (colonized) | | | p-value^{c} | | p-value^{c} |
| | | Fold | p-value^{c} | Fold | (vs. WU1 | Fold | (vs.WU1Δspa |
| | Antigens | change^{b} | (vs. naïve) | change^{b} | colonized) | change^{b} | colonized) |
| Cell | SpA_{KKAA} | 1.3 ± 0.08 | n.s | 1.1 ± 0.06 | n.s. | 1.1 ± 0.49 | n.s. |
| wall | ClfA | 5.3 ± 2.77 | <0.0001 | 4.3 ± 0.83 | n.s. | 3.5 ± 1.69 | n.s. |
| anchored | ClfB | 4.8 ± 0.72 | 0.001 | 3.9 ± 1.28 | n.s. | 17.4 ± 4.70 | <0.0001 |
| surface | Ebh | 3.7 ± 0.50 | 0.0454 | 2.8 ± 0.62 | n.s. | 3.9 ± 1.56 | n.s. |
| protein | FnbpA | 1.9 ± 0.89 | n.s. | 1.3 ± 0.79 | n.s. | 2.6 ± 0.96 | n.s. |
| | FnbpB | 2.6 ± 1.33 | n.s. | 2.3 ± 0.85 | n.s. | 4.3 ± 0.96 | n.s. |
| | IsdA | 4.5 ± 0.84 | 0.0036 | 2.1 ± 0.22 | n.s. | 13.0 ± 0.44 | <0.0001 |
| | IsdB | 5.2 ± 1.43 | 0.0002 | 2.7 ± 0.83 | n.s. | 2.8 ± 1.18 | n.s. |
| | SdrC | 1.1 ± 0.14 | n.s. | 1.5 ± 0.45 | n.s. | 1.7 ± 0.69 | n.s. |
| | SdrD | 1.5 ± 1.08 | n.s. | 1.0 ± 0.25 | n.s. | 1.2 ± 0.35 | n.s. |
| | SdrE | 1.8 ± 0.52 | n.s. | 2.9 ± 0.65 | n.s. | 1.4 ± 0.60 | n.s. |
| | SasA | 3.0 ± 1.33 | n.s. | 1.1 ± 0.44 | n.s. | 3.3 ± 1.14 | n.s. |
| | SasB | 5.1 ± 2.22 | n.s. | 1.0 ± 0.34 | n.s. | 5.7 ± 4.42 | n.s. |
| | SasD | 2.7 ± 1.47 | n.s. | 0.7 ± 0.23 | n.s. | 1.3 ± 0.59 | n.s. |
| | SasF | 1.2 ± 0.61 | n.s. | 0.9 ± 0.63 | n.s. | 1.2 ± 0.32 | n.s. |
| | SasG | 2.1 ± 0.24 | n.s. | 1.2 ± 0.47 | n.s. | 10.3 ± 1.19 | <0.0001 |
| | SasI | 1.4 ± 0.75 | n.s. | 1.2 ± 0.08 | n.s. | 1.4 ± 0.53 | n.s. |
| | SasK | 2.5 ± 0.26 | n.s. | 1.3 ± 0.30 | n.s. | 1.7 ± 1.09 | n.s. |
| Secreted | Coa | 2.7 ± 0.29 | n.s. | 1.2 ± 0.45 | n.s. | 1.5 ± 0.45 | n.s. |
| protein | vWbp | 2.0 ± 0.97 | n.s. | 1.4 ± 0.59 | n.s. | 1.7 ± 0.89 | n.s. |
| | Hla | 1.8 ± 0.65 | n.s. | 1.2 ± 0.46 | n.s. | 1.2 ± 0.34 | n.s. |
| | SCIN | 4.3 ± 1.23 | 0.0071 | 2.8 ± 1.80 | n.s. | 1.4 ± 0.49 | n.s. |
| | Eap | 1.3 ± 0.20 | n.s. | 0.8 ± 0.97 | n.s. | 1.2 ± 0.31 | n.s. |
| | Efb | 2.9 ± 1.68 | n.s. | 2.6 ± 1.63 | n.s. | 1.6 ± 0.52 | n.s. |
| | EsxA | 2.6 ± 1.73 | n.s. | 1.6 ± 1.00 | n.s. | 2.6 ± 0.35 | n.s. |
| | EsxB | 2.8 ± 0.28 | n.s. | 1.6 ± 0.19 | n.s. | 1.9 ± 0.21 | n.s. |

^{a}Cohorts of C57BL/6J mice were inoculated intra-nasally with 10⁸ CFU of indicated S. aureus strains. 15 days following inoculation, animals were bled and serum samples were analyzed for antibody responses to staphylococcal antigens.
^{b}Fold changes of were calculated by dividing the average signal intensities of inoculated mouse group by the average signal intensities of naïve mouse group. Data are presented in means ± standard deviation.
^{c}p-values were calculated using Two-way ANOVA with Tukey multiple comparison tests, n.s. = not significant

S. aureus WU1 requires staphylococcal protein A for persistent colonization.

Similar to S. aureus Newman SpA, the spa gene product of S. aureus WUT is comprised of five IgBDs and carries a single amino acid substitution within the 278-residue domain. Immunoblotting experiments revealed that S. aureus strains Newman and WU1 produced similar amounts of SpA (FIG. 3A). Using allelic recombination, the inventors generated the Δspa mutant of S. aureus WU1. As measured by immunoblotting, SpA production was abolished in the Δspa mutant and this defect was restored by plasmid-borne expression of wild-type spa (pSpA)(FIG. 3A). Immunoblotting with antibodies against sortase A (SrtA) was used as a loading control (FIG. 3A). When inoculated into the right nostril of mice and analyzed for colonization by oropharyngeal swab on day 7, the Δspa mutant initially colonized C57BL/6J animals in a manner similar to wild-type strain WU1 (FIG. 3B). However, at later time points, particularly on day 35 and 42, the Δspa mutant colonized fewer animals than wild-type strain WU1 (FIG. 3B). During bacterial growth, S. aureus releases SpA-linked to peptidoglycan fragments into the surrounding milieu (46). In a mouse model of intravenous S. aureus challenge, released SpA activates B cell proliferation and enhanced secretion of VH3 idiotype IgM and IgG molecules (33). However, expanded VH3 idiotype IgG do not recognize staphylococcal antigens (33). The molecular basis for this B cell superantigen activity is based on SpA-mediated crosslinking of VH3 idiotype B cell receptors, which triggers B cell proliferation in a CD4 T helper cell and RIPK2 kinase dependent manner (33, 47). Animals infected with Δspa mutant staphylococci lack VH3 idiotypic immunoglobulin expansion and exhibit increased abundance of pathogen-specific IgG, thereby triggering immune responses that are protective against subsequent S. aureus infection (48). It was then wondered whether colonization with the Δspa mutant of WU1 was associated with altered serum IgG responses. Sera from animals that had been colonized for 15 days were analyzed for IgG binding to components of the S. aureus antigen matrix (TABLE 3). This experiment revealed increases in antibodies against ClfB, IsdA and SasG in animals that subsequently decolonized, but not in animals that remained colonized with the Δspa mutant (TABLE 3). Taken together, these data suggest that nasopharyngeal colonization of C57BL/6 mice with Δspa mutant staphylococci is associated with increased IgG responses against key colonization determinants, which appears to promote removal of Δspa mutant S. aureus from the nasopharynx.

Protein A-Neutralizing Antibodies Affect Persistent Colonization with S. aureus.

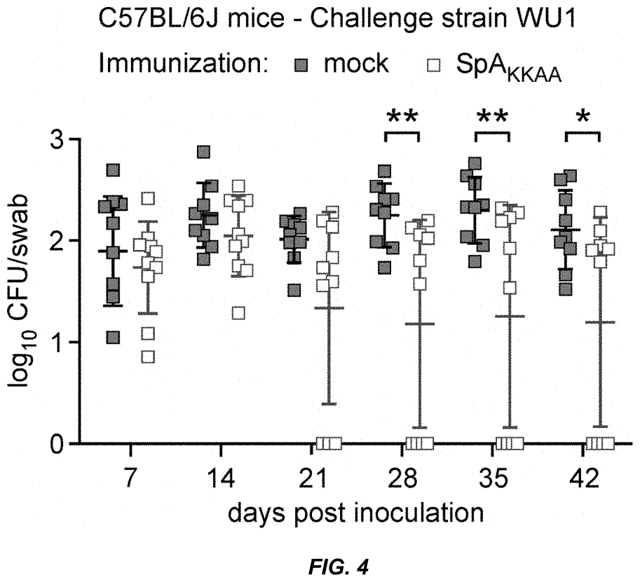

Immunization of mice with wild-type protein A does not elicit IgG serum antibodies that bind and neutralize the capacity of its five IgBDs to bind either the Fcγ domain of IgG molecules or the variant heavy chain of VH3 idiotype immunoglobulin (44). SpA_{KKAA} is a variant with 20 amino acid substitutions throughout the five IgBDs of SpA that abolish Fcγ binding and also diminish association with VH3 idiotype immunoglobulin (44). Nevertheless, SpA_{KKAA} retains the overall α-helical content and antigen structure of protein A. As a result, immunization of mice with adjuvanted SpA_{KKAA} elicits high-titer protein A neutralizing IgG (44). These antibodies block the anti-opsonic and B cell superantigen activities of protein A during S. aureus infection, broadly enhancing IgG responses against staphylococcal antigens and promoting the development of protective immunity (44). To test whether or not protein A-neutralizing antibodies affect S. aureus colonization, C57BL/6 mice were immunized with adjuvanted SpA_{KKAA} or with adjuvant alone. Compared to mock immunized animals, SpA_{KKAA} treated animals elicited high titer protein A neutralizing antibodies (TABLE 4). When inoculated with S. aureus WU1, both mock and SpA_{KKAA} immunized animals were initially colonized in a similar manner, as oropharyngeal swabs revealed average colonizing loads that were not significantly different on days 7 and 14 following inoculation (FIG. 4). However, beginning on day 21, SpA_{KK}ax immunized mice were more frequently decolonized than mock-immunized animals (FIG. 4). When examined for serum IgG responses and compared naïve mice, S. aureus WU1 colonization in mock treated animals led to antibody responses against ClfB, IsdA, IsdB, SasD and SasF (TABLE 4). In animals that maintained S. aureus WU1 colonization, SpA_{KKAA} immunization led to antibody responses against ClfA, Coa, vWBP, and Hla (TABLE 4). As compared to SpA_{KKAA} vaccinated C57BL/6J mice, animals that subsequently decolonized exhibited elevated serum IgG against ClfA, ClfB, fibronectin binding proteins A (FnBPA) and B (FnBPB), IsdB, Coa, and SasG (TABLE 4). Together these data indicate that SpA_{KKAA} vaccination elicits enhanced serum IgG responses in mice that had been colonized with S. aureus. Further, SpA_{KKAA} vaccine induced antibodies against many different staphylococcal antigens, including known colonization factors (ClfB, IsdA and SasG). Together, these SpA_{KKAA} vaccine induced IgG responses against colonizing staphylococci appear to promote decolonization of the nasopharynx.

TABLE 4
Impact of SpA_{KKAA} immunization on serum IgG responses in S. aureus WU1 colonized
C57BL/6 mice

| | | | | SpA_{KKAA} immunized | | | |
|---|---|---|---|---|---|---|---|
| | | | | (cleared) | | | |
| | | SpA_{KKAA} immunized | | | p-value^{d} | | |
| | | (colonized) | | | (vs. | PBS mock immunized | |
| | | | p-value^{d} | | SpA_{KKAA} | (colonized) | |
| | | Fold | (vs. PBS | Fold | immunized | Fold | p-value^{d} |
| | Antigens | change^{b} | mock) | change^{b} | colonized) | change^{c} | (vs. naïve |
| Cell | SpA_{KKAA} | 121.3 ± 64.98 | <0.0001 | 126.3 ± 13.35 | <0.0001 | 0.9 ± 0.16 | n.s. |
| wall | ClfA | 3.8 ± 0.49 | <0.0001 | 5.7 ± 2.28 | 0.0069 | 1.3 ± 0.65 | n.s. |
| anchored | ClfB | 1.1 ± 0.28 | n.s. | 14.8 ± 1.12 | <0.0001 | 4.3 ± 1.49 | <0.0001 |
| surface | Ebh | 1.0 ± 0.15 | n.s. | 1.3 ± 0.57 | n.s. | 1.3 ± 0.43 | n.s. |
| protein | FnbpA | 1.1 ± 0.34 | n.s. | 6.4 ± 1.86 | <0.0001 | 1.1 ± 0.29 | n.s. |
| | FnbpB | 1.5 ± 0.33 | n.s. | 10.6 ± 1.0 | <0.0001 | 1.2 ± 0.72 | n.s. |
| | IsdA | 1.8 ± 0.46 | n.s. | 2.8 ± 0.59 | n.s. | 2.0 ± 0.43 | n.s. |
| | IsdB | 1.7 ± 0.37 | n.s. | 5.8 ± 2.75 | <0.0001 | 2.1 ± 0.96 | n.s. |
| | SdrC | 1.4 ± 0.67 | n.s. | 1.5 ± 0.61 | n.s. | 1.2 ± 0.45 | n.s. |
| | SdrD | 1.1 ± 0.39 | n.s. | 1.5 ± 0.36 | n.s. | 1.2 ± 0.23 | n.s. |
| | SdrE | 1.2 ± 0.36 | n.s. | 1.8 ± 0.94 | n.s. | 1.2 ± 0.22 | n.s. |
| | SasA | 1.8 ± 0.36 | n.s. | 1.6 ± 0.28 | n.s. | 0.8 ± 0.80 | n.s. |
| | SasB | 1.9 ± 0.90 | n.s. | 1.1 ± 0.42 | n.s. | 1.0 ± 0.24 | n.s. |
| | SasD | 1.3 ± 0.46 | n.s. | 1.0 ± 0.44 | n.s. | 2.4 ± 0.53 | 0.0023 |
| | SasF | 2.4 ± 0.34 | n.s. | 1.7 ± 0.55 | n.s. | 2.6 ± 1.59 | 0.004 |
| | SasG | 0.9 ± 0.15 | n.s. | 5.5 ± 1.04 | <0.0001 | 1.1 ± 0.32 | n.s. |
| | SasI | 2.1 ± 0.46 | n.s. | 1.8 ± 0.02 | n.s. | 1.3 ± 0.22 | n.s. |
| | SasK | 2.3 ± 0.62 | n.s. | 2.7 ± 0.38 | n.s. | 1.1 ± 0.02 | n.s. |
| Secreted | Coa | 3.0 ± 1.31 | 0.0049 | 5.8 ± 0.87 | <0.0001 | 1.2 ± 0.43 | n.s. |
| protein | vWbp | 5.7 ± 1.34 | <0.0001 | 6.6 ± 2.82 | n.s. | 1.4 ± 0.65 | n.s. |
| | Hla | 2.9 ± 0.08 | 0.0070 | 3.6 ± 0.36 | n.s. | 1.1 ± 0.58 | n.s. |
| | SCIN | 2.1 ± 0.77 | n.s. | 1.4 ± 0.21 | n.s. | 1.0 ± 0.37 | n.s. |
| | Eap | 1.7 ± 0.38 | n.s. | 1.1 ± 0.22 | n.s. | 0.9 ± 0.23 | n.s. |
| | Efb | 1.5 ± 0.47 | n.s. | 1.49 ± 0.25 | n.s. | 0.98 ± 0.27 | n.s. |
| | EsxA | 2.4 ± 0.65 | n.s. | 3.22 ± 1.81 | n.s. | 0.82 ± 0.26 | n.s. |
| | EsxB | 2.5 ± 0.35 | n.s. | 3.75 ± 1.08 | n.s. | 1.46 ± 0.25 | n.s. |

^{a}Cohorts of C57BL/6J mice were immunized with 50 μg of recombinant SpA_{KKAA} emulsified with CFA or PBS-mock in CFA, and on day 11 boosted with 50 μg of recombinant SpA_{KKAA} emulsified with IFA or PBS-mock in IFA. On day 24, the mice were inoculated intra-nasally with 10⁸ CFU of indicated S. aureus strains and were swabbed in the throat weekly to enumerate the bacterial load. 15 days following inoculation, animals were bled and serum samples were analyzed for antibody responses to staphylococcal antigens.
^{b}Fold changes of were calculated by dividing the average signal intensities of SpA_{KKAA}-immunized group by the average signal intensities of PBS mock-immunized group. Data are presented in means ± standard deviation.

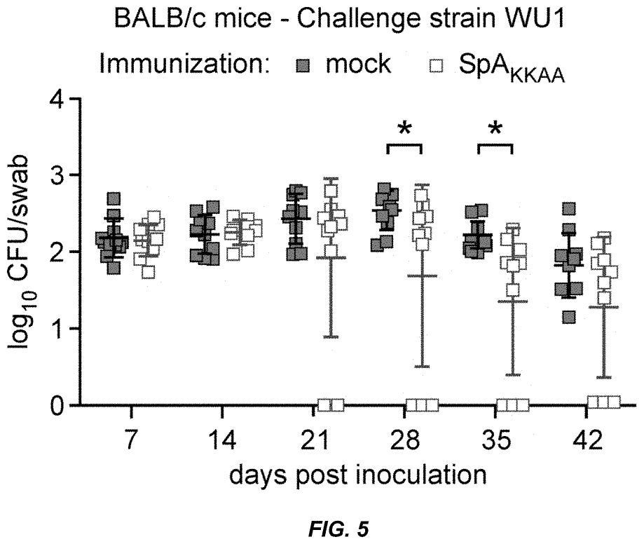

S. aureus WU1 colonization of BALB/c mice. To test whether S. aureus WU1 colonization was restricted to C57BL/6 mice, the inventors inoculated cohorts (n=20) of naïve BALB/c mice with 1×10⁸ CFU S. aureus WU1 into the right nostril and measured nasopharyngeal colonization with swab cultures. Similar to C57BL/6 mice, S. aureus WU1 persistently colonized BALB/c mice (FIG. 5). Immunization of BALB/c mice with SpA_{KKAA} did not affect the initial colonization with S. aureus WU1. However, when compared to mock immunized animals, vaccination with SpA_{KKAA} promoted decolonization of BALB/c mice (FIG. 5).

SpA_{KKAA} Vaccine Affects Mouse Colonization with S. aureus JSNZ.

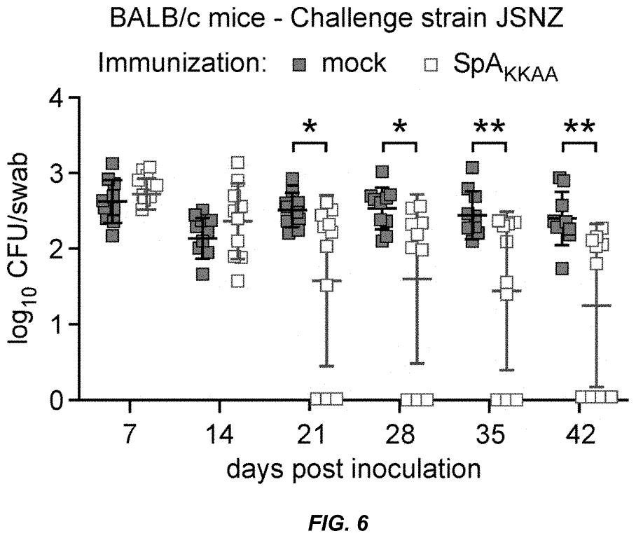

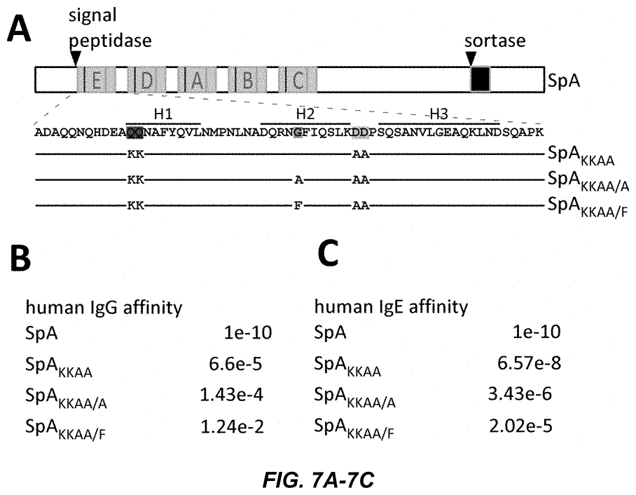

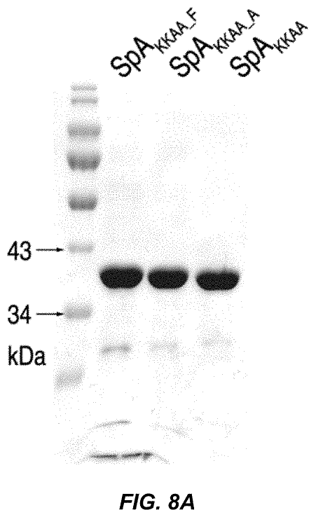

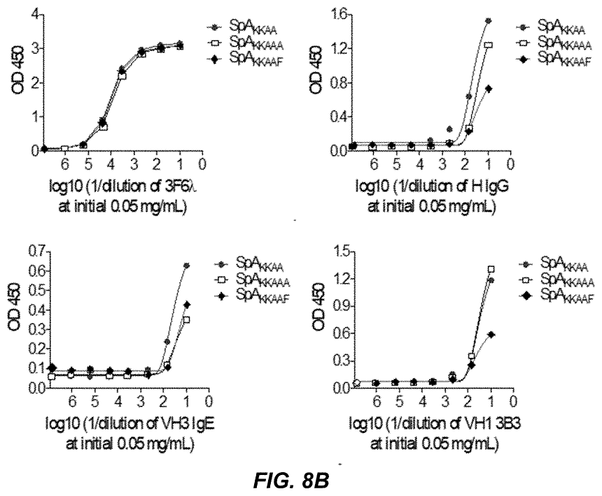

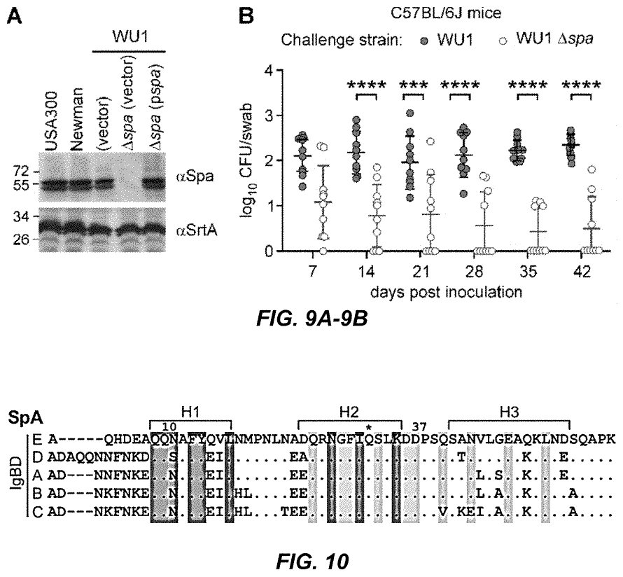

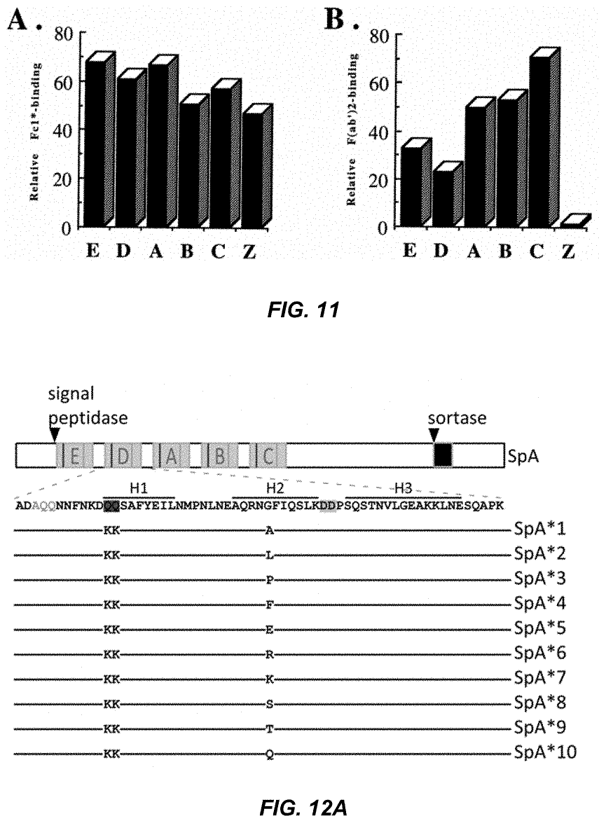

It was then wondered whether or not protein A-neutralizing antibodies affect also mouse colonization with S. aureus JSNZ. Unlike strains Newman and WU1, the spa gene product of S. aureus JSNZ comprises only four IgBDs (37). Earlier work demonstrated that SpA variants with four IgBDs are associated with diminished B cell superantigen activity, as compared to the five IgBDs generally associated with S. aureus colonization of the human nasopharynx (33). When inoculated into the right nostril of anesthetized mice, S. aureus JSNZ effectively colonized the nasopharynx of BALB/c mice over 42 days (FIG. 6). SpA_{KKAA} vaccination did not affect initial colonization with S. aureus JSNZ. However, as compared to mock immunized mice, BALB/c mice with serum neutralizing protein A antibodies more frequently decolonized S. aureus JSNZ starting on day 21 (FIG. 6). Together these data suggest that S. aureus JSNZ also requires protein A-mediated B cell superantigen activity for persistent colonization of mice.

Materials and Methods

Media and Bacterial Growth Conditions.

S. aureus strains were propagated in tryptic soy broth (TSB) or on tryptic soy agar (TSA) at 37° C. For experiments investigating mouse nasopharyngeal colonization, throat swab samples were grown on Baird-Parker agar at 37° C. as indicated. For experiments investigating S. aureus GI tract colonization, stool samples were grown on Mannitol Salt agar at 37° C. as indicated. Escherichia coli strains DH5α and BL21 (DE3) were grown in Luria broth (LB) or agar at 37° C. Ampicillin (100 μg/ml for E. coli) and chloramphenicol (10 μg/ml for S. aureus) were used for plasmid selection.

S. aureus Genotyping.

S. aureus isolate WU1 was obtained from the nasopharynx and preputial gland abscess lesions of mice in the inventors' animal facility. Mouse S. aureus strain JSNZ was provided by Dr. Siouxsie Wiles (36). Staphylococcal genomic DNA was isolated with the Wizard Genomic DNA Purification Kit (Promega). Spa genotyping and multilocus sequence typing (MLST) were performed as previously described (85). Briefly, for spa typing, the genomic DNA of S. aureus strain WU1 was PCR amplified with primers 1095F (5′AGACGATCCTTCGGTGAGC3′) (SEQ ID NO:89) and 1517R (5′GCTTTTGCAATGTCATTTACTG3′) (SEQ ID NO:90) (86). The PCR product was purified with the Nucleospin Gel and PCR Clean-up kit, sequenced with primers 1095F and 1517R, and analyzed with the Ridom software. For MLST typing, the genomic DNA of S. aureus strain WU1 was PCR amplified with primers arc-up (5′TTGATTCACCAGCGCGTATTGTC3′) (SEQ ID NO:91), arc-dn (5′AGGTATCTGCTTCAATCAGCG3′) (SEQ ID NO:92), aro-up (5′ATCGGAAATCCTATTTCACATTC3′) (SEQ ID NO:93), arc-dn (5′GGTGTTGTATTAATAACGATATC3′) (SEQ ID NO:94), glp-up (5′CTAGGAACTGCAATCTTAATCC3′) (SEQ ID NO:95), glp-dn (5′TGGTAAAATCGCATGTCCAATTC3′) (SEQ ID NO:96), gmk-up (5′ATCGTTTTATCGGGACCATC3′) (SEQ ID NO:97), gmk-dn (5′TCATTAACTACAACGTAATCGTA3′) (SEQ ID NO:98), pta-up (5′GTTAAAATCGTATTACCTGAAGG3′) (SEQ ID NO:99), pta-dn (5′GACCCTTTTGTTGAAAAGCTTAA3′) (SEQ ID NO:100), tpi-up (5′TCGTTCATTCTGAACGTCGTGA3′) (SEQ ID NO: 101), tpi-dn (5′TTTGCACCTTCTAACAATTGTAC3′) (SEQ ID NO:102), yqi-up (5′CAGCATACAGGACACCTATTGGC3′) (SEQ ID NO:103) and yqi-dn (5′CGTTGAGGAATCGATACTGGAAC3′) (SEQ ID NO:104) (see for example: saureus.mlst.net/misc/info.asp). The PCR product was purified with the Nucleospin Gel and PCR Clean-up kit, PCR amplified and sequenced and analyzed with the on-line software (see, for example: saures.mlst.net/). Whole genome sequence files for S. aureus strain JSNZ were provided by Dr. Silva Holtfreter. Truseq DNA-seq library preparation Illumina MiSeq sequencing were performed with the genomic DNA of S. aureus WU1 by the Environmental Sample Preparation and Sequencing Facility at Argonne National Laboratory. Sequence were analyzed using the Geneious software.

S. aureus Mutants.

Allelic recombination with the plasmid pKOR1 was used to delete the spa gene of S. aureus WU1 (87). To construct the Δspa mutant, two 1-kb DNA fragment upstream and downstream of the spa gene were amplified from the chromosome of S. aureus WU1 with primers ext1F ext1F (5′ GGGGACCACTTTGTACAAGAAAGCTGGGTCATTTAAGAAGATTGTTT CAGATTTATG 3′) (SEQ ID NO:105), ext1R (5′ ATTTGTAAAGTCATCATAATATAAC GAATTATGTATTGCAATACTAAAATC 3′) (SEQ ID NO:106), ext2F (5′ CGTCGCG AACTATAATAAAAACAAACAATACACAACGATAGATATC 3′) (SEQ ID NO:107), and ext2R (5′ GGGGACAAGTTTGTACAAAAAAGCAGGCAACGAACGCCTAAAGAAATTGT CTTTGC 3′) (SEQ ID NO:108). The two flanking regions were fused together in a subsequent PCR, and final PCR product was cloned into pKOR1 using the BP Clonase II kit (Invitrogen). The resulting plasmid were consecutively transferred into E. coli DH5α, S. aureus strain RN4220, and finally S. aureus strain WU1 and temperature shifted to 40° C., blocking replication of plasmids and promoting their insertion into the chromosome (87). Growth at 30° C. was used to promote allelic replacement. Mutations in the spa genes were verified by DNA sequencing of PCR amplification products.

Agglutination Assay.

Agglutination assays were performed as previously described (88). Briefly, Overnight cultures of S. aureus strains were diluted 1:100 in fresh TSB and grown at 37° C. for 6 hours. Bacteria from 1 ml culture (normalized to OD₆₀₀ 4.0) was incubated with SYTO 9 (1:500) (Invitrogen) for 15 min, washed twice with 1 ml PBS, and suspended in 1 ml PBS. Bacteria were mixed 1:1 with citrate-treated human plasma or mouse plasma on glass microscope slides and incubated for 30 min. Samples were viewed and images were captured on an IX81 live cell total internal reflection fluorescence microscope using a 20× objective (Olympus). At least 10 images were acquired for each sample. The areas of agglutination complexes in each image were measured and quantified using ImageJ software.

Immunoblotting.

Overnight cultures of S. aureus strains were diluted 1:100 into fresh TSB (with chloramphenicol in the presence of plasmids) and grown at 37° C. to OD₆₀₀ 0.5-1.0. Cells from 1 ml culture were centrifuged, suspended in PBS and incubated with 20 μg/ml lysostaphin (AMBI) at 37° C. for 1 h. Proteins in the whole cell lysate were precipitated with 10% trichloracetic acid and 10 μg deoxycholic acid, washed with ice-cold acetone, air-dried, suspended in 100 μl 0.5M Tris HCl (pH 6.8) and 100 μl SDS-PAGE sample buffer [100 mM Tris HCl (pH 6.8), 4% SDS, 0.2% bromophenol blue, 200 mM dithiothreitol] and boiled for 10 min. Proteins were separated on 12% SDS-PAGE and electrotransferred to PVDF membrane. PVDF membranes were blocked with 5% milk in Tris Buffered Saline with Tween-20 (TBST) [20 mM Tris HCl (pH 7.6), 137 mM NaCl, 0.1% Tween-20]. Mouse anti-ClfA 2A12.12 monoclonal antibody (1:2,000 dilution) and horseradish peroxidase (HRP)-conjugated anti-mouse IgG (Cell Signaling, 1:10,000 dilution) were used to detect ClfA. Rabbit anti-Coa polyclonal antibody (1:1,000 dilution) and HRP-conjugated anti-rabbit IgG (1:10,000 dilution) were used to detect Coa. Two different rabbit anti-vWbp polyclonal antibodies (1:1,000 dilution), which recognize full length vWbp from S. aureus Newman or the C terminal domain of vWbp, respectively, and HRP-conjugated anti-rabbit IgG (1:10,000 dilution) were used to detect vWbp. HRP-conjugated human IgM in TBST (1:10,000 dilution) was used to detect SpA. Rabbit anti-SrtA polyclonal antibodies (1:10,000 dilution) and HRP-conjugated anti-rabbit IgG (1:10,000 dilution) were used to detect SrtA. Antibody-stained membranes were washed with TBST and incubated with SuperSignal West Pico Chemiluminescent Substrate (Thermo Scientific) and developed onto Amersham Hyperfilm ECL high performance chemiluminescence films (GE Healthcare).

Purification of Recombinant Proteins.

E. coli BL21(DE3) harboring pET15b+ plasmids for the expression of His-tagged SpA_{KKAA}, as well as 24 staphylococcal antigens (ClfA, ClfB, FnBPA, FnBPB, IsdA, IsdB, SasA, SasB, SasD, SasF, SasG, SasI, SasK, SdrC, SdrD, SdrE, EsxA, EsxB, SCIN, Eap, Efb, Hla, Coa, vWbp, and Ebh), was grown overnight, diluted 1:100 in fresh medium, and grown at 37° C. to ˜OD₆₀₀ of 0.5. Cultures were induced with 1 mM isopropyl-β-d-thiogalactopyranoside and grown for an additional 3 h. Cells were pelleted, re-suspended in column buffer (50 mM Tris-HCl [pH 7.5], 150 mM NaCl), and disrupted with a French pressure cell at 14,000 lb/in2 Lysates were cleared of membrane and insoluble components by ultracentrifugation at 40,000×g. Cleared lysates were subjected to Ni-NTA affinity chromatography, and proteins were eluted in column buffer containing successively higher concentrations of imidazole (100 to 500 mM). Eluates were dialyzed with PBS, and the protein purity was verified by Coomassie-stained SDS-PAGE. Protein concentrations were determined by bicinchoninic acid assay (Thermo Scientific).

Mouse Nasopharyngeal Colonization.

Overnight cultures of S. aureus strains WU1 and its Δspa mutant were diluted 1:100 into fresh TSB and grown for 2 h at 37° C. Cells were centrifuged, washed and suspended in PBS. Seven-week-old female BALB/c, C57BL/6J or B6.129S2-Ighm^{tm1Cgn}/J mice (The Jackson Laboratory) were anesthetized by intraperitoneal injection with 100 mg/ml ketamine and 20 mg/ml xylazine per kilogram of body weight. 1×10⁸ CFU of S. aureus (in 10 μl volume) were pipetted into the right nostril of each mouse. On day 7, 14, 21, 28, 35, and 42 following inoculation, the oropharynx of mice was swabbed, and swab samples spread on Baird-Parker agar and incubated for bacterial enumeration. On day 15 following the inoculation, the mice were bled via periorbital vein puncture to obtain sera for antibody response analyses using the staphylococcal antigen matrix. On day 42 following inoculation, stool samples were collected and homogenized in PBS. The homogenates were plated on Mannitol Salt agar and incubated for bacterial enumeration. All mouse experiments were performed in accordance with the institutional guidelines following experimental protocol review and approval by the Institutional Biosafety Committee (IBC) and the Institutional Animal Care and Use Committee (IACUC) at the University of Chicago. Animals experiments were repeated at least once to ensure reproducibility of the data.

Active Immunization.

Four-week-old mice were immunized by subcutaneous injection with 50 μg of SpA_{KKAA} emulsified in complete Freund's adjuvant (CFA; Difco) and boosted with 50 μg of the same antigen emulsified in incomplete Freund's adjuvant (IFA) 11 days following the initial immunization. On day 21, immunized mice were bled via periorbital vein puncture to obtain sera for ELISA. On day 24, the mice were inoculated intranasally with 1×10⁸ CFU of S. aureus strains WU1 or JSNZ and monitored for nasopharyngeal colonization.

Staphylococcal Antigen Matrix.

Nitrocellulose membranes were blotted with 2 μg affinity-purified staphylococcal antigens. Membranes were blocked with 5% degranulated milk, incubated with diluted mouse sera (1:10,000 dilution) and IRDye 680-conjugated goat anti-mouse IgG (LI-COR). Signal intensities were quantified using the Odyssey infrared imaging system (LI-COR).

Statistical Analysis.

Two-way ANOVA with Sidak multiple comparison tests (GraphPad Software) was performed to analyze the statistical significance of nasopharyngeal colonization, ELISA, and antigen matrix data.

### Example 2: Staphylococcal Protein A Variants

The following assays can be used to evaluate SpA variants described herein for their efficacy in the methods and compositions of the disclosure.

Assays

Vaccine protection in murine abscess, murine lethal infection, and murine pneumonia models. Three animal models have been established for the study of S. aureus infectious disease. These models can be used here to examine the level of protective immunity provided via the generation of Protein A specific antibodies.

Murine abscess—BALB/c mice (24-day-old female, 8-10 mice per group, Charles River Laboratories, Wilmington, Mass.) can be immunized by intramuscular injection into the hind leg with purified protein (Chang et al., 2003; Schneewind et al., 1992). Purified SpA and/or SpA variant can be administered on days 0 (emulsified 1:1 with complete Freund's adjuvant) and 11 (emulsified 1:1 with incomplete Freund's adjuvant). Blood samples can be drawn by retroorbital bleeding on days 0, 11, and 20. Sera can be examined by ELISA for IgG titers for specific binding activity of the variant. Immunized animals can be challenged on day 21 by retroorbital injection of 100 μl of S. aureus Newman or S. aureus USA300 suspension (1×10⁷ cfu). For this, overnight cultures of S. aureus Newman can be diluted 1:100 into fresh tryptic soy broth and grown for 3 h at 37° C. Staphylococci can be centrifuged, washed twice, and diluted in PBS to yield an A₆₀₀ of 0.4 (1×10⁸ cfu per ml). Dilutions can be verified experimentally by agar plating and colony formation. Mice can be anesthetized by intraperitoneal injection of 80-120 mg of ketamine and 3-6 mg of xylazine per kilogram of body weight and infected by retroorbital injection. On day 5 or 15 following challenge, mice can be euthanized by compressed CO₂ inhalation. Kidneys can be removed and homogenized in 1% Triton X-100. Aliquots can be diluted and plated on agar medium for triplicate determination of cfu. For histology, kidney tissue can be incubated at room temperature in 10% formalin for 24 h. Tissues can be embedded in paraffin, thin-sectioned, stained with hematoxylinleosin, and examined by microscopy.

Murine lethal infection—BALB/c mice (24-day-old female, 8-10 mice per group, Charles River Laboratories, Wilmington, Mass.) can be immunized by intramuscular injection into the hind leg with purified SpA or SpA variant. Vaccine can be administered on days 0 (emulsified 1:1 with complete Freund's adjuvant) and 11 (emulsified 1:1 with incomplete Freund's adjuvant). Blood samples can be drawn by retroorbital bleeding on days 0, 11, and 20. Sera are examined by ELISA for IgG titers with specific binding activity of the variant. Immunized animals can be challenged on day 21 by retroorbital injection of 100 μl of S. aureus Newman or S. aureus USA300 suspension (15×10⁷ cfu). For this, overnight cultures of S. aureus Newman can be diluted 1:100 into fresh tryptic soy broth and grown for 3 h at 37° C. Staphylococci can be centrifuged, washed twice, diluted in PBS to yield an A₆₀₀ of 0.4 (1×10⁸ cfu per ml) and concentrated. Dilutions can be verified experimentally by agar plating and colony formation. Mice can be anesthetized by intraperitoneal injection of 80-120 mg of ketamine and 3-6 mg of xylazine per kilogram of body weight. Immunized animals can be challenged on day 21 by intraperitoneal inject with 2×10¹⁰ cfu of S. aureus Newman or 3-10×109 cfu of clinical S. aureus isolates. Animals can be monitored for 14 days, and lethal disease can be recorded.

Murine pneumonia model—S. aureus strains Newman or USA300 (LAC) can be grown at 37° C. in tryptic soy broth/agar to OD₆₆₀ 0.5. 50-ml culture aliquots can be centrifuged, washed in PBS, and suspended in 750 μl PBS for mortality studies (3-4×10⁸ CFU per 30-1 volume), or 1,250 μl PBS (2×10⁸ CFU per 30-1 volume) for bacterial load and histopathology experiments. For lung infection, 7-wk-old C57BL/6J mice (The Jackson Laboratory) can be anesthetized before inoculation of 30 μl of S. aureus suspension into the left nare. Animals can be placed into the cage in a supine position for recovery and observed for 14 days. For active immunization, 4-wk-old mice can receive 20 μg SpA variant in CFA on day 0 via the i.m. route, followed by a boost with 20 μg of variant in incomplete Freund's adjuvant (IFA) on day 10. Animals can be challenged with S. aureus on day 21. Sera can be collected before immunization and on day 20 to assess specific antibody production. For passive immunization studies, 7-wk-old mice can receive 100 μl of either NRS (normal rabbit serum) or SpA-variant-specific rabbit antisera via i.p. injection 24 h before challenge. To assess the pathological correlates of pneumonia, infected animals can be killed via forced CO₂ inhalation before removal of both lungs. The right lung can be homogenized for enumeration of lung bacterial load. The left lung can be placed in 1% formalin and paraffin embedded, thin sectioned, stained with hematoxylin-eosin, and analyzed by microscopy.

Rabbit antibodies—Purified SpA variant can be used as an immunogen for the production of rabbit antisera. Protein can be emulsified with CFA for injection at day 0, followed by booster injections with protein emulsified with IFA on days 21 and 42. Rabbit antibody titers can be determined by ELISA. Purified antibodies can be obtained by affinity chromatography of rabbit serum on SpA variant sepharose. The concentration of eluted antibodies can be measured by absorbance at A₂₈₀ and specific antibody titers can be determined by ELISA.

Active immunization with SpA-variants.—To determine vaccine efficacy, animals can be actively immunized with purified SpA variant. As a control, animals can be immunized with adjuvant alone. Antibody titers against Protein A preparations can be determined using SpA variant as antigens. Using infectious disease models described above, any reduction in bacterial load (murine abscess and pneumonia), histopathology evidence of staphylococcal disease (murine abscess and pneumonia) and protection from lethal disease (murine lethal challenge and pneumonia) can be measured.

Passive immunization with affinity purified rabbit polyclonal antibodies generated against SpA-variants. To determine protective immunity of Protein A specific rabbit antibodies, mice are passively immunized with purified SpA variant derived rabbit antibodies. Both of these antibody preparations are purified by affinity chromatography using immobilized SpA variant. As a control, animals are passively immunized with rV10 antibodies (a plague protective antigen that has no impact on the outcome of staphylococcal infections). Antibody titers against all Protein A preparations are determined using SpA variant as an antigen. Using the infectious disease models described above, the reduction in bacterial load (murine abscess and pneumonia), histopathology evidence of staphylococcal disease (murine abscess and pneumonia), and the protection from lethal disease (murine lethal challenge and pneumonia) can be measured.

Bacterial strains and growth. Staphylococcus aureus strains Newman and USA300 can be grown in tryptic soy broth (TSB) at 37° C. Escherichia coli strains DH5α and BL21 (DE3) can be grown in Luria-Bertani (LB) broth with 100 μg ml⁻¹ ampicillin at 37° C.

Rabbit Antibodies. The SpA variants can be made according to standard recombinant technology or synthesis protocols, and purified antigen can be covalently linked to HiTrap NHS-activated HP columns (GE Healthcare). Antigen-matrix can be used for affinity chromatography of 10-20 ml of rabbit serum at 4° C. Charged matrix can be washed with 50 column volumes of PBS, antibodies eluted with elution buffer (1 M glycine, pH 2.5, 0.5 M NaCl) and immediately neutralized with 1M Tris-HCl, pH 8.5. Purified antibodies can be dialyzed overnight against PBS at 4° C.

F(ab)₂ fragments. Affinity purified antibodies can be mixed with 3 mg of pepsin at 37° C. for 30 minutes. The reaction can be quenched with 1 M Tris-HCl, pH 8.5 and F(ab)₂ fragments can be affinity purified with specific antigen-conjugated HiTrap NHS-activated HP columns. Purified antibodies can be dialyzed overnight against PBS at 4° C., loaded onto SDS-PAGE gel and visualized with Coomassie Blue staining.

Active and passive immunization. BALB/c mice (3 week old, female, Charles River Laboratories) can be immunized with 50 μg protein emulsified in Complete Freund's Adjuvant (Difco) by intramuscular injection. For booster immunizations, proteins can be emulsified in Incomplete Freund's Adjuvant and injected 11 days following the initial immunization. On day 20 following immunization, 5 mice can be bled to obtain sera for specific antibody titers by enzyme-linked immunosorbent assay (ELISA).

Affinity purified antibodies in PBS can be injected at a concentration 5 mg kg⁻¹ of experimental animal weight into the peritoneal cavity of BALB/c mice (6 week old, female, Charles River Laboratories) 24 hours prior to challenge with S. aureus. Animal blood can be collected via periorbital vein puncture. Blood cells can be removed with heparinized micro-hematocrit capillary tubes (Fisher) and Z-gel serum separation micro tubes (Sarstedt) can be used to collect and measure antigen specific antibody titers by ELISA.

Mouse renal abscess. Overnight cultures of S. aureus Newman or USA300 (LAC) can be diluted 1:100 into fresh TSB and grown for 2 hours at 37° C. Staphylococci can be sedimented, washed and suspended PBS at OD₆₀₀ of 0.4 (˜1×10⁸ CFU ml-1). Inocula can be quantified by spreading sample aliquots on TSA and enumerating colonies formed. BALB/c mice (6 week old, female, Charles River Laboratories) can be anesthetized via intraperitoneal injection with 100 mg ml-ketamine and 20 mg ml⁻¹ xylazine per kilogram of body weight. Mice can be infected by retro-obital injection with 1×10⁷ CFU of S. aureus Newman or 5×10⁶ CFU of S. aureus USA300. On day 4 following challenge, mice can be killed by CO₂ inhalation. Both kidneys can be removed, and the staphylococcal load in one organ can be analyzed by homogenizing renal tissue with PBS, 1% Triton X-100. Serial dilutions of homogenate were spread on TSA and incubated for colony formation. The remaining organ can be examined by histopathology. Briefly, kidneys can be fixed in 10% formalin for 24 hours at room temperature. Tissues can be embedded in paraffin, thin-sectioned, stained with hematoxylin-eosin, and inspected by light microscopy to enumerate abscess lesions. All mouse experiments can be performed in accordance with the institutional guidelines following experimental protocol review and approval by the Institutional Biosafety Committee (IBC) and the Institutional Animal Care and Use Committee (IACUC) at the University of Chicago.

Protein A binding. For human IgG binding, Ni-NTA affinity columns can be pre-charged with 200 μg of purified proteins (SpA variants) in column buffer. After washing, 200 μg of human IgG (Sigma) can be loaded onto the column. Protein samples can be collected from washes and elutions and subjected to SDS-PAGE gel electrophoresis, followed by Coomassie Blue staining. Purified proteins (SpA variants) can be coated onto MaxiSorp ELISA plates (NUNC) in 0.1M carbonate buffer (pH 9.5) at 1 μg ml-1 concentration overnight at 4° C. Plates can next be blocked with 5% whole milk followed by incubation with serial dilutions of peroxidase-conjugated human IgG, Fc or F(ab)₂ fragments for one hour. Plates can be washed and developed using OptEIA ELISA reagents (BD). Reactions can be quenched with 1 M phosphoric acid and A₄₅₀ readings were used to calculate half maximal titer and percent binding.

von Willebrand Factor (vWF) binding assays. Purified proteins (SpA variants) can be coated and blocked as described above. Plates can be incubated with human vWF at 1 μg ml-1 concentration for two hours, then washed and blocked with human IgG for another hour. After washing, plates can be incubated with serial dilution of peroxidase-conjugated antibody directed against human vWF for one hour. Plates can be washed and developed using OptEIA ELISA reagents (BD). Reactions can be quenched with 1 M phosphoric acid and A₄₅₀ readings can be used to calculate half maximal titer and percent binding. For inhibition assays, plates can be incubated with affinity purified F(ab)₂ fragments specific for a SpA- variant at 10 μg ml⁻¹ concentration for one hour prior to ligand binding assays.

Splenocyte apoptosis. Affinity purified proteins (150 μg of SpA variant) can be injected into the peritoneal cavity of BALB/c mice (6 week old, female, Charles River Laboratories). Four hours following injection, animals were killed by CO₂ inhalation. Their spleens can be removed and homogenized. Cell debris can be removed using cell strainer and suspended cells can be transferred to ACK lysis buffer (0.15 M NH₄Cl, 10 mM KHCO₃, 0.1 mM EDTA) to lyse red blood cells. White blood cells can be sedimented by centrifugation, suspended in PBS and stained with 1:250 diluted R-PE conjugated anti-CD19 monoclonal antibody (Invitrogen) on ice and in the dark for one hour. Cells can be washed with 1% FBS and fixed with 4% formalin overnight at 4° C. The following day, cells can be diluted in PBS and analyzed by flow cytometry. The remaining organ can be examined for histopathology. Briefly, spleens can be fixed in 10% formalin for 24 hours at room temperature. Tissues can be embedded in paraffin, thin-sectioned, stained with the Apoptosis detection kit (Millipore), and inspected by light microscopy.

Antibody quantification. Sera can be collected from healthy human volunteers or BALB/c mice that had been either infected with S. aureus Newman or USA300 for 30 days or that had been immunized with an SpA variant as described above. Human/mouse IgG (Jackson Immunology Laboratory), SpA variant, and CRM₁₉₇ can be blotted onto nitrocellulose membrane. Membranes can be blocked with 5% whole milk, followed by incubation with either human or mouse sera. IRDye 700DX conjugated affinity purified anti-human/mouse IgG (Rockland) can be used to quantify signal intensities using the Odyssey™ infrared imaging system (Li-cor). Experiments with blood from human volunteers involved protocols that were reviewed, approved and performed under regulatory supervision of The University of Chicago's Institutional Review Board (IRB).

Statistical Analysis. Two tailed Student's t tests can be performed to analyze the statistical significance of renal abscess, ELISA, and B cell superantigen data.

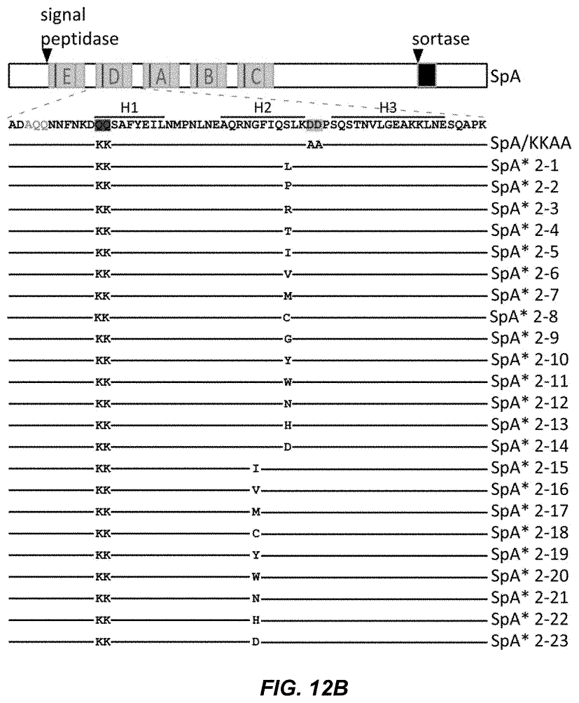

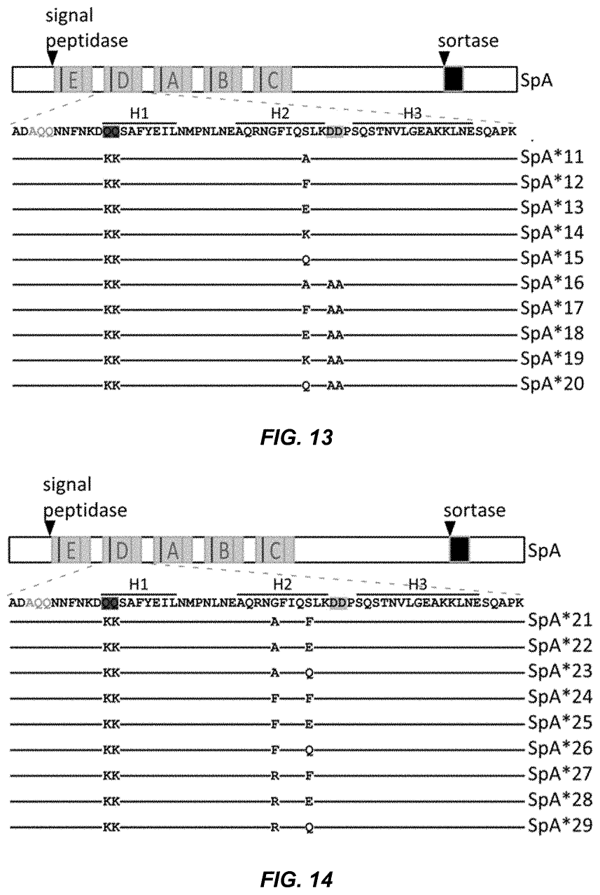

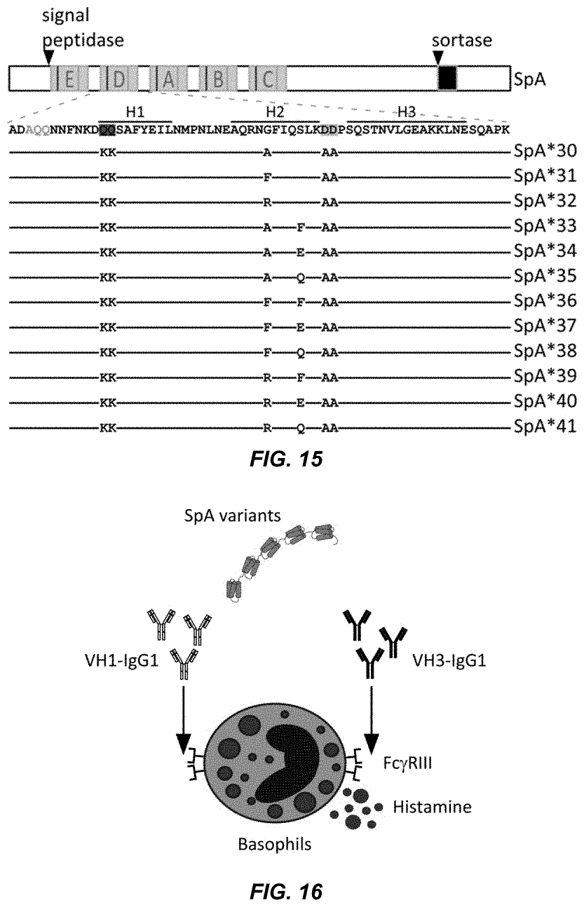

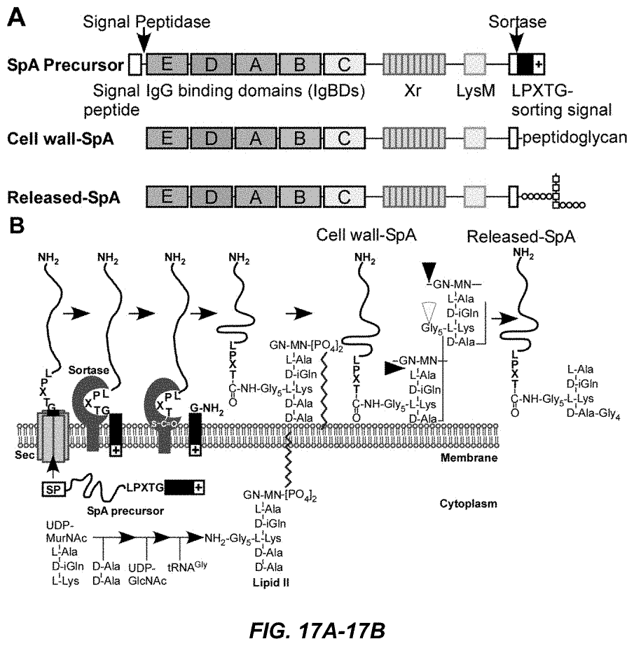

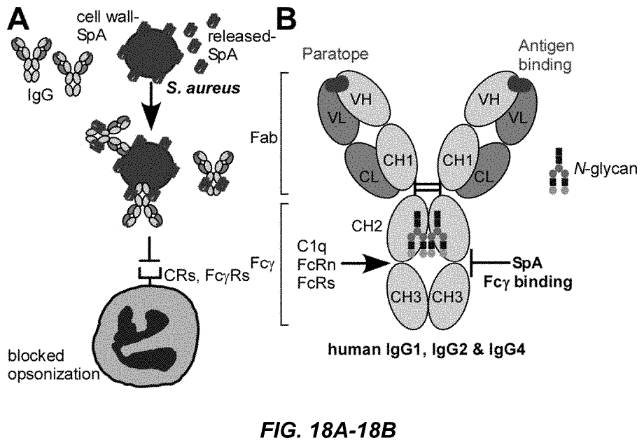

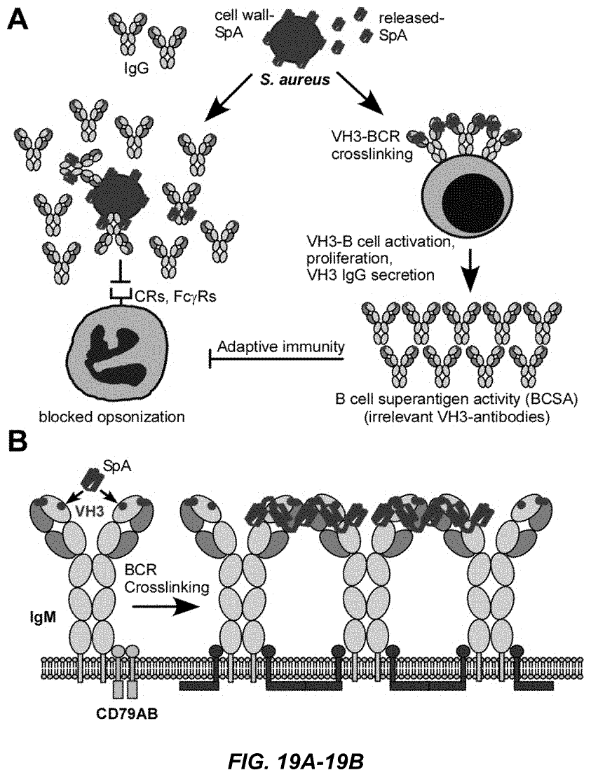

Using these assays, the variants described herein (e.g. those shown in FIGS. 12-15) can be tested. Further assays can be performed, such as a SPR analysis to determine the binding affinities of new SpA variants with human VH3-IgG and human VH3-IgE compared to SpA, SpA/KKAA as well as SpA/KKAA/F (SpA*31) controls. The manufacturability (the yield of purified SpA* variants/gram of E. coli cell paste) can also be tested. CD spectroscopy can be performed to test the α-helical content in comparison with SpA and SpA/KKAA. Protein stability during purification and storage at variable temperature (4, 25 and 37 C for 1-7 days) can also be determined.

To test for drug safety and efficacy, a basil histamine release assay may be performed (FIG. 16). This test is known in the art (see, for example, Kowal, K. et al., 2005. Allergy and Asthma Proc. Vol. 26, No. 6). Briefly, human serum and/or basophils can be incubated for 60 min. at 37° C. Histamine release can be measured from stimulated (by addition of SpA variants) and unstimulated cells and the results can expressed as histamine release in percentage of the total histamine content. In some aspects, a histamine release >16.5% is a positive test result in both children and adult patients.

### Example 3: Spa Vaccine Variants with Improved Safety

Results

Amino Acid Substitutions at Gly²⁹ of SpA Vaccine Candidates

The inventors sought to experimentally identify amino acid substitutions at position Gly²⁹ of the SpA-IgBDs that cause the greatest reduction in affinity between human IgG and SpA_{KK}, i.e. five IgBDs (EDABC) carrying also the amino acid substitutions Gln^{9,10}Lys, which disrupt the interaction between SpA and Fcγ (48). Towards this goal, the inventors constructed nineteen different plasmids encoding N-terminally polyhistidine-tagged SpA_{Q9,10K/G29X}, where X is any one of the 19 natural amino acids (except glycine) provided by the genetic code. SpA_{Q9,10K/G29X} proteins were purified via affinity chromatography on Ni-NTA resin, eluted, dialyzed, concentration determined via the BCA assay and bound at equal concentration (250 nM) to Bio-Rad ProteOn HTGchip. Each chip was subjected to surface plasmon resonance experiments with serial dilutions of human IgG or PBS control. The association of human IgG with SpA vaccine candidates loaded on the chip were recorded and data transformed to derive the association constants for each protein (Table 5). As control, the inventors quantified the association constants of wild-type SpA (K_{A} 1.081×10⁸ M⁻¹) and SpA_{KK}AA for human IgG (K_{A} 5.022×10⁵ M⁻¹). For SpA_{Q9,10K/G29X} proteins, four amino acid substitutions at Gly²⁹ caused a significant increase in the association constant: Gly²⁹Ser (K_{A} 9.398×10¹ M⁻¹), Gly²⁹Lys (Kₐ9.738×10⁵ M⁻¹), Gly²⁹Ile (K_{A} 10.070×10⁵ M⁻¹) and Gly²⁹Ala (K_{A} 11.310×10⁵ M⁻¹), suggesting that these variants bound more tightly to the VH3-variant heavy chains of human IgG than SpA_{KKAA} (Table 5). The observations for SpA_{Q9,10K/G29A} were surprising to us. The Gly²⁹Ala substitution in the ZZZZ construct for commercial antibody purification (MabSelectSure™) diminishes binding to V_{H}3-IgG (150), whereas Gly²⁹Ala in the context of Gln^{9,10}Lys within SpA-IgBDs may promote a modest increase the affinity for V_{H}3-IgG. As compared with SpA_{KKAA}, ten amino acid substitutions at Gly²⁹ did not cause a significant difference in the association constant with: Gly²⁹Thr, Gly²⁹Leu, Gly²⁹Glu, Gly²⁹Pro, Gly²⁹Phe, Gly²⁹Met, Gly²⁹Val, Gly²⁹Trp, Gly²⁹Asp, Gly²⁹Arg, Gly²⁹Asn, and Gly²⁹Tyr (Table 5). Another three amino acid substitutions at Gly²⁹ reduced the association constant: Gly²⁹His (Kₐ 1.435×10⁵ M⁻¹), Gly²⁹Cys (Kₐ 1.743×10⁵ M⁻¹), and Gly²⁹Gln (Kₐ 2.057×10⁵ M⁻¹) to human IgG as compared to SpA_{KKAA} (Table 4). Thus, amino acid substitutions at Gly²⁹ do not exert a universal effect on the binding of SpA-IgBDs to human IgG. Some amino acid substitutions at Gly²⁹ increase the affinity between human IgG and SpA_{Q9,10K/G29X}, whereas others are either neutral (exert no significant effect) or diminish the affinity.

Amino Acid Substitutions at Ser33 of SpA Vaccine Candidates

To identify amino acid substitutions at position Ser³³ of the SpA-IgBDs that cause the greatest reduction in affinity between human IgG and SpA_{KK}, the inventors constructed nineteen different plasmids encoding N-terminally polyhistidine-tagged SpA_{Q9,10K/S33X}, where X is any one of the 19 natural amino acids (except serine) provided by the genetic code. SpA_{Q9,10K/S33X} proteins were purified via affinity chromatography on Ni-NTA resin, eluted, dialyzed, concentration determined via the BCA assay and bound at equal concentration (250 nM) to Bio-Rad ProteOn HTG chip. Each chip was subjected to surface plasmon resonance experiments with serial dilutions of human IgG and PBS control. The association of human IgG with SpA vaccine candidates loaded on the chip were recorded and data transformed to derive association constants for each protein (Table 5). Two amino acid substitutions at Ser³³ caused an increase in affinity for human IgG: Ser³³Gly (K_{A} 11.180×10⁵ M⁻¹) and Ser³³Ala (K_{A} 10.540×10⁵ M⁻¹), indicating that these variants exhibit greater affinity for human IgG than SpA_{KKAA} (presumably due to increased affinity for V_{H}3-variant heavy chains) (Table 6). Fourteen amino acid substitutions at Ser³³ did not cause a significant difference in the association constant: Ser³³Tyr, Ser³³Leu, Ser³³Trp, Ser³³Val, Ser³³His, Ser³³Asn, Ser³³Met, Ser³³Arg, Ser³³Δsp, Ser³³Phe, Ser³³Gln, Ser³³Pro, Ser³³Cys and Ser³³Lys (Table 5). Three amino acid substitutions at Ser³³ decreased the affinity for human IgG and SpA_{Q9,10K/S33X}: Ser³³Thr (K_{A} 0.386×10⁵ M⁻¹), Ser³³Glu (K_{A} 0.496×10⁵ M⁻¹), and Ser³³Ile (K_{A} 1.840×10⁵ M⁻¹) (Table 6). Thus, some amino acid substitutions at Ser³³ increase the affinity between human IgG and SpA_{Q9,10K/S33X}, whereas others are either neutral (exert no significant effect) or diminish the association with human IgG. Of those that diminish the affinity between human IgG, Ser³³Glu and Ser³³Thr, exhibit the largest reduction in the association constant (Table 5).

Combining Amino Acid Substitutions at Gly29, Ser33 and Asp36,37 in SpA Vaccine Candidates

As compared to a single amino acid substitution at Ser³³, do combinations of amino acid substitutions at positions Gly²⁹, Ser³³ or Asp^{36,37} of the IgBDs cause further affinity reductions for human IgG or do multiple substitutions exert paradoxical effects that can also increase the affinity between the two proteins? To address this question, the inventors compared the association constants of three proteins with amino acid substitutions at Ser³³: SpA_{Q9,10K/S33E} (decreased affinity), SpA_{Q9,10K/S33F} (affinity unaffected), and SpA_{Q9,10K/S33Q} (affinity unaffected)—with those carrying additional amino acid substitutions at Gly²⁹ and/or Asp^{36,37} (Table 6). For SpA_{Q9,10K/S33E} (K_{A} 0.496×10⁵ M⁻¹), no additional effect was observed with added substitutions Gly²⁹Ala (K_{A} 1.265×10⁵ M⁻¹), Gly²⁹Phe (K_{A} 1.575×10⁵ M⁻¹), Asp^{36,37}Ala (K_{A} 0.568×10⁵ M⁻¹), Gly²⁹Ala/Asp^{36,37}Ala (K_{A} 1.892×10⁵ M⁻¹) or Gly²⁹Arg (K_{A} 4.840×10⁵ M⁻¹). However, combining Asp^{36,37}Ala with either Gly²⁹Phe (K_{A} 14.850×10⁵ M⁻¹) or Gly²⁹Arg (K_{A} 10.240×10⁵ M⁻¹) increased the affinity of SpA_{Q9,10K/S33E} for human IgG (Table 7). When analyzed for SpA_{Q9,10K/S33F} (K_{A} 3.902×10⁵ M⁻¹), whose association constant is not significantly different from that of SpA_{KKAA}, the inventors observed similar effects. None of the substitutions altered the affinity of SpA_{Q9,10K/S33F} for human IgG except when Asp^{36,37}Ala was combined with either Gly²⁹Phe (SpA_{Q9,10K/S33Q/D36,37A/Gly29F} K_{A} 12.470×10⁵ M⁻¹) which here again increased the affinity of the parent vaccine for human IgG (Table 7). Thus, combining amino acid substitutions at Gly²⁹, Ser³³ and Asp^{36,37} of the SpA-IgBDs does not predictably reduce the affinity for human IgG. In each case, the affinity of a recombinant SpA vaccine candidate needs to be experimentally determined.

SpA-KR is a variant of SpA_{KKAA} with two additional amino acid substitutions in the E domain of the IgBD, which carries a six residue N-terminal extension with the amino acid sequence ADAQQN (International Patent Application WO 2015/144653 A1). The inventors—Fabio Bagnoli, Luigi Fiaschi and Maria Scarselli (Glaxo-SmithKline INC.)—speculated that the two glutamine (QQ) residues in the hexapeptide extension of the E domain of SpA_{KKAA} may constitute an additional binding site for human IgG without specifying where these residues may bind to immunoglobulin, i.e. Fcγ or V_{H}3-heavy chains, or providing experimental proof for such binding. When analyzed for its affinity to human IgG, the association constant of SpA-KR (K_{A}5.464×10⁵ M⁻¹) was not significantly different from that of SpA_{KKAA}, suggesting that SpA-KR may also exhibit crosslinking activity for V_{H}3-IgG (Table 7). SpA_{RRVV} is a SpA vaccine variant that is described in the patent application EP3101027A1 (OLYMVAX INC.). Similar to SpA_{KKAA}, SpA_{RRVV} harbors amino acid substitutions at Gln^{9,10} and Asp^{36,37} of each of the five IgBDs of SpA, albeit that the substitutions replace Gln^{9,10} with arginine (Arg or R) and Asp^{36,37} with valine (Val or V). When analyzed for its affinity to human IgG, the association constant of SpA_{RRVV} (K_{A} 5.609×10⁵ M⁻¹) was similar to that of SpA_{KKAA}, suggesting that SpA_{RRVV} may also exhibit crosslinking activity for V_{H}3-IgG (Table 7).

Crosslinking Activity of SpA Vaccine Variants for VH3-Idiotypic and Fab Fragments of Human IgG

A key safety issue for the clinical development of SpA vaccines is the lack of crosslinking activity with V_{H}3-idiotypic IgE and IgG on the surface of basophils and mast cells, which otherwise triggers histamine release and anaphylaxis (140, 142, 145). To quantify the V_{H}3-crosslinking activity of SpA vaccine candidates, the inventors used purified human IgG (54% V_{H}3 idiotypic variant heavy chains) that had been cleaved with papain and V_{H}3-clonal Fab fragments purified using affinity chromatography on SpA_{KK} (75) (Table 8). When examined using Surface Plasmon Resonance (SPR) for affinity measurements with SpA and its variants, the IgBDs of wild-type protein A (SpA) exhibited potent crosslinking activity (K_{A} 1.44×10⁷ M⁻¹, Table 8). The affinity for V_{H}3-Fab was diminished for SpA_{KKAA} (K_{A} 8.27×10⁴ M⁻¹), and SpA-KR (K_{A} 6.42×10⁴ M⁻¹) albeit that both variants retained significant crosslinking activity when compared to SpA_{Q9,10K/S33E} (K_{A} 41.24 M⁻¹) and SpA_{Q9,10/S33T} (K_{A} 43.55 M⁻¹) (Table 8). SpA_{Q9,10K/S33E} and SpA_{Q9,10/S33T} exhibited similar binding properties as PBS control (i.e. values obtained when no ligand was added). Thus, amino acid substitutions Ser³³Glu and Ser³³Thr eliminate V_{H}3-IgE and V_{H}3-IgG crosslinking activities in the vaccine candidates SpA_{Q9,10K/S33E} and SpA_{Q9,10/S33T}, respectively.

Fcγ-Binding Activity of SpA Vaccine Variants

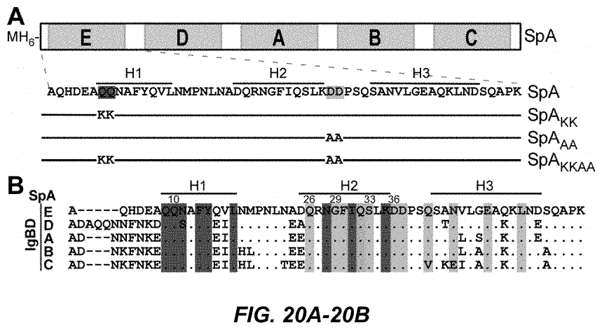

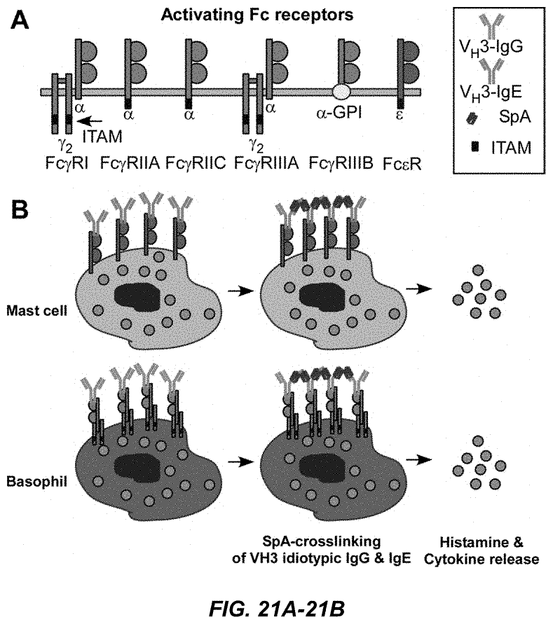

Deisenhofer solved the crystal structure of the SpA B domain (IgBD-B) bound to human Fcγ and identified the interface between the two molecules (154). Four hydrogen bonds promote interactions between SpA (B domain numbering, FIG. 20B) and Fcγ: Gln⁹ (IgG Ser²⁵⁴), Gln¹⁰ (IgG Gln³¹¹), Asn¹¹ (IgG Asn⁴³⁴) and Tyr¹⁴ (IgG Leu⁴³²)(54). These B domain residues are conserved in all five IgBDs (FIG. 20), implying a universal mechanism of Fcγ binding(43). Earlier work showed that substitution of Gln^{9,10}Lys in IgBD-D or in all five IgBDs of SpA diminishes SpA_{KK} (SpA_{Q9,10K}) binding to human, mouse and guinea pig IgG Fcγ (76, 43). As the newly engineered SpA vaccine variants, SpA_{Q9,10K/S33E} and SpA_{Q9,10K/S33T}, retain the Gln^{9,10} Lys amino acid substitutions in their five IgBDs, the inventors surmised that these variants should also exhibit significant defects in binding to human Fcγ. To validate this conjecture, the inventors used purified human IgG that had been cleaved with papain and the resulting Fcγ fragments purified (Table 9). When examined using a Bio-Layer Interferometer (BLI) for affinity measurements with SpA and its variants, the IgBDs of wild-type protein A exhibited high affinity for Fcγ (K_{A} 5.17×10⁷ M⁻¹). The Fey-binding activity was abolished for SpA_{KKAA} (K_{A} 32.68 M⁻¹), SpA-KR (K_{A} 39.12 M⁻¹), SpA_{Q9,10K/S33E} (K_{A} 32.68 M⁻¹) and SpA_{Q9,10K/S33T}(K_{A} 39.91 M⁻¹), respectively. Thus, the Ser³³Glu and Ser³³Thr substitutions do not perturb the effects of the Gln^{9,10}Lys on Fey-binding in helix 1 of SpA_{Q9,10K/S33E} and SpA_{Q9,10K/S33T} (Table 9).

Mouse Model for Anaphylactic Activity of SpA Vaccine Candidates

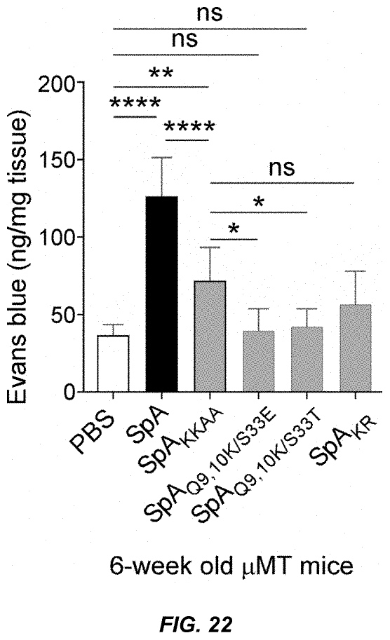

Clinical and experimental studies have shown that vascular hyperpermeability is the hallmark of anaphylaxis (155, 156). Activated mast cells or basophils release vasoactive mediators, including histamine and platelet-activating factor, which induce the anaphylactic response of vascular hyperpermeability by causing vasodilation and endothelial barrier disruption (156). These events can be measured in a mouse model of anaphylactic vascular hyperpermeability as the extravasation of an intravenously administered dye, Evans Blue, at experimental sites (ear tissue) primed 24-hour prior via intradermal injection of 2 μg human V_{H}3-idiotypic IgG (157). The vascular leakage of Evans Blue into ear tissue is subsequently quantified (ng dye/mg tissue) in cohorts of five animals, means and standard deviation (SD) calculated, and data analyzed for statically significant differences. The plasma of wild-type C57BL/6 mice contains only 5-10% of immunoglobulin with V_{H}3-idiotypic variant heavy chains (48). For this reason, mice, unlike guinea pigs (20-30% V_{H}3-idiotypic variant heavy chains), are resistant to SpA-induced anaphylactic shock (140). The inventors therefore chose MT mice for their study; these animals lack functional IgM B cell receptors, arrest B cell development at the pre-B cell stage, and cannot produce plasma IgG (158). MT mice were used as recipients for the intradermal injection of 2 μg human V_{H}3-idiotypic IgG into ear tissue. After 24 hours, 200 μg SpA, SpA vaccine variants or buffer control (PBS) were injected intravenously into mice. Five minutes following SpA treatment, 2% Evans Blue solution was injected intravenously into mice to assess vascular permeability in ear tissues. After 30 min, animals were euthanized, ear tissue excised, dried and extracted with formamide for spectrophotometric quantification of the dye. Compared with PBS control [34.73 (±) 8.474 ng Evans Blue/mg ear tissue], SpA treatment caused anaphylactic vascular hyperpermeability, releasing 124.9 ng/mg (±26.54 ng/mg) Evans Blue (PBS vs. SpA, P<0.0001) (FIG. 22). In animal cohorts pretreated by intradermal injection with human V_{H}3-IgG, intravenous administration of SpA_{KKAA} also caused vascular hyperpermeability [70.31 ng/mg (±23.04 ng/mg); PBS vs. SpA_{KKAA}, P<0.01], albeit at a lower level than wild-type SpA (SpA vs. SpA_{KK}AA, P<0.0001) (FIG. 22). In contrast, intravenous administration of 200 g SpA_{Q9,10K/S33E} [38.57 ng/mg (±15.07 ng/mg); SpA_{Q9,10K/S33E} vs. PBS, not significant] or SpA_{Q9,10K/S33T} [41.43 ng/mg (±13.15 ng/mg); SpA_{Q9,10K/S33T} vs. PBS, not significant] did not elicit vascular hyperpermeability at sites treated with V_{H}3-idiotypic human IgG in MT mice (FIG. 22). As a comparison, the SpA-KR vaccine candidate elicited anaphylactic vascular hyperpermeability similar to that of SpA_{KKAA} (FIG. 22). Thus, unlike SpA and SpA_{KKAA}, which trigger vascular hyperpermeability by crosslinking V_{H}3-idiotypic IgG bound to activating FcεRI on mast cells and basophils or FcγR on other effector cells, SpA_{Q9,10K/S33E} and SpA_{Q9,10K/S33T} cannot crosslink V_{H}3-idiotypic IgG to promote anaphylactic reactions in MT mice at sites pretreated with V_{H}3-idiotypic human IgG.

SpA Vaccine Candidate Crosslinking of V_{H}3-IgE

Basophils and mast cells are two main effector cells of anaphylaxis responses and secrete proinflammatory mediators upon antigen-mediated cross-linking of IgE onto their FcεRI surface receptors. S. aureus Cowan I strain that expresses SpA in abundance or soluble purified SpA can activate basophils to induce histamine release. This stimulating effect is dependent on the Fab binding activity of protein A (145). To study the potential crosslinking effect of SpA vaccine candidates with circulating IgE or IgG bound on the surface of basophils, vaccine variants purified in PBS were added to freshly drawn human blood anti-coagulated with EDTA for 30 min. Wild-type SpA was used as a positive control. PBS was used as the negative control. Cells were stained with anti-CD123, anti-CD203c, anti-HLA-DR (removal of dendritic cells and monocytes) and anti-CD63. Basophils were identified by gating for SSC^{low}CD203c⁺/CD123⁺/HLA-DR⁻ cells. CD123 basophil activation was expressed as a proportion of CD63, and corrected for negative and positive controls. Compared with PBS control (4.39% activated basophil), SpA or SpA_{KKAA} treatments caused significant increases of CD63⁺ activated basophil population, 32.05% (PBS vs. SpA, P<0.0001) and 10.66% (PBS vs. SpA_{KKAA}, P<0.01), respectively (Table 10). In contrast to SpA_{KKAA}, SpA_{Q9,10K/S33E} [5.38%; SpA_{Q9,10K/S33T} vs. SpA_{KKAA}, P<0.05] or SpA_{Q9,10K/S33T} [4.57%; SpA_{Q9,10K/S33T} vs. SpA_{KKAA}, P<0.01] were unable to activate basophils and behaved similar to PBS control (Table 10). In this assay, SpA-KR [8.15%] and SpA_{RRVV} [10.16%] vaccine candidates showed similar basophil activation as SpA_{KKAA}. Thus, SpA_{Q9,10K/S33E} and SpA_{Q9,10K/S33T} cannot crosslink circulating IgE in blood and cannot sensitize basophils by binding the high affinity receptors FcεRI. Unlike SpA_{Q9,10K/S33E} and SpA_{Q9,10K/S33}, the SpA_{KKAA}, SpA-KR and SpA_{RRVV} vaccine candidates retain significant activity for IgE-crosslinking which initiate an unwanted systemic anaphylaxis reaction.

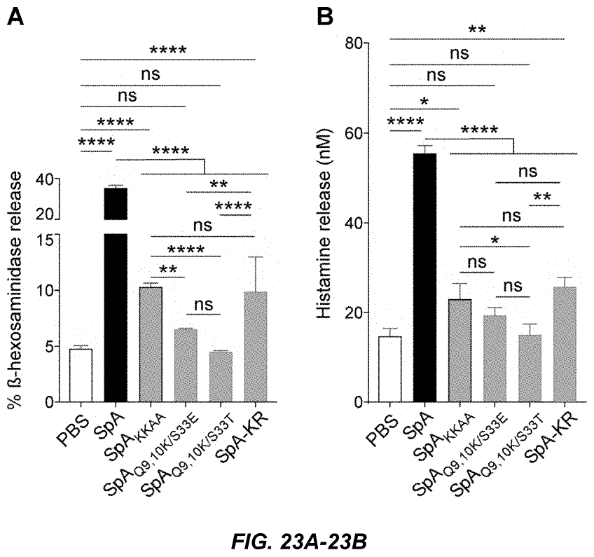

Mast cell functional response was measured by antigen-triggered β-hexosaminidase and histamine release. The human mast cell line LAD2 was used for this assay. Mast cells (2×10⁵ cells/ml) were sensitized following overnight incubation with 100 ng/ml VH3 IgE prior to stimulation with SpA vaccine variants (10 μg) for 30 min and β-hexosaminidase (FIG. 23A) or histamine release (FIG. 23B) were measured. Incubation with wild-type SpA induced about 35% of 3-hexosaminidase release. SpA_{KKAA} and SpA-KR vaccines caused 10.32% and 9.87% of β-hexosaminidase release, respectively, with no significant difference (SpA-KR vs. SpA_{KKAA}, not significant). These reductions are significant when compared to SpA wild-type (SpA vs. SpA_{KKAA}, P<0.0001; SpA vs. SpA-KR, P<0.0001). Yet, SpA_{KKAA} and SpA-KR vaccines retain β-hexosaminidase releasing activity above negative control levels (SpA_{KK}AA vs. PBS, P<0.0001; SpA-KR vs. PBS, P<0.0001) (FIG. 23A). In comparison, SpA_{Q9,10K/S33E} [6.46%; SpA_{Q9,10K/S33E} vs. SpA_{KKAA}, P<0.01] and SpA_{Q9,10K/S33T} [4.43%; SpA_{Q9,10K/S33T} vs. SpA_{KKAA}, P<0.0001] caused significantly less β-hexosaminidase release as compared to SpA_{KKAA}. SpA_{Q9,10K/S33E} and SpA_{Q9,10K/S33T} exhibited similar β-hexosaminidase release as the PBS control (FIG. 23A).

Similar results were obtained when assessing histamine release (FIG. 23B). SpA stimulated the highest level of histamine release; SpA_{KKAA} and SpA-KR vaccines retained histamine release activity above PBS control levels, and both SpA_{Q9,10K/S33E} and SpA_{Q9,10/S33T} behaved like the negative control PBS [SpA vs. PBS, or SpA_{KKAA}, or SpA-KR, or SpA_{Q9,10K/S33E}, or SpA_{Q9,10K/S33T}, P<0.0001; SpA_{KKAA} vs SpA-KR or SpA_{Q9,10K/S33E}, not significant; SpA_{KKAA} vs. SpA_{Q9,10K/S33T} or PBS, P<0.05; SpA_{Q9,10K/S33T} vs. SpA-KR, P<0.01].

In conclusion, SpA_{Q9,10K/S33E} and SpA_{Q9,10K/S33T} have lost the ability to activate mast cells sensitized with V_{H}3-idiotypic IgE, and represent vaccine candidates with a safety profile appropriate for human clinical testing.

Immunogenicity and Efficacy of SpA Vaccine Candidates in the S. aureus Colonization Model

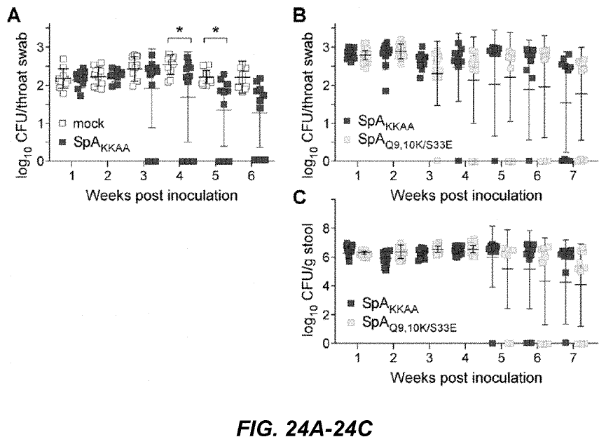

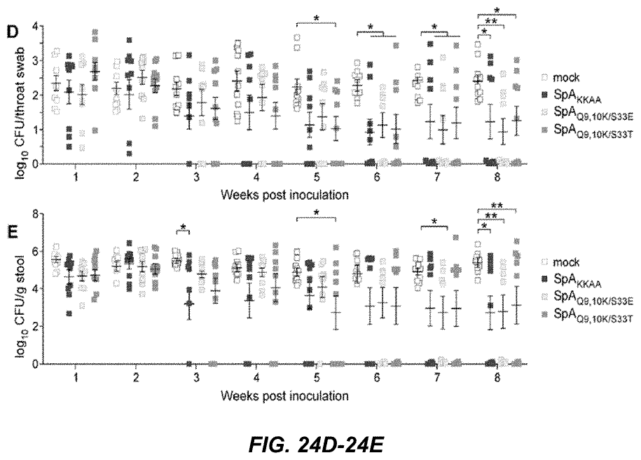

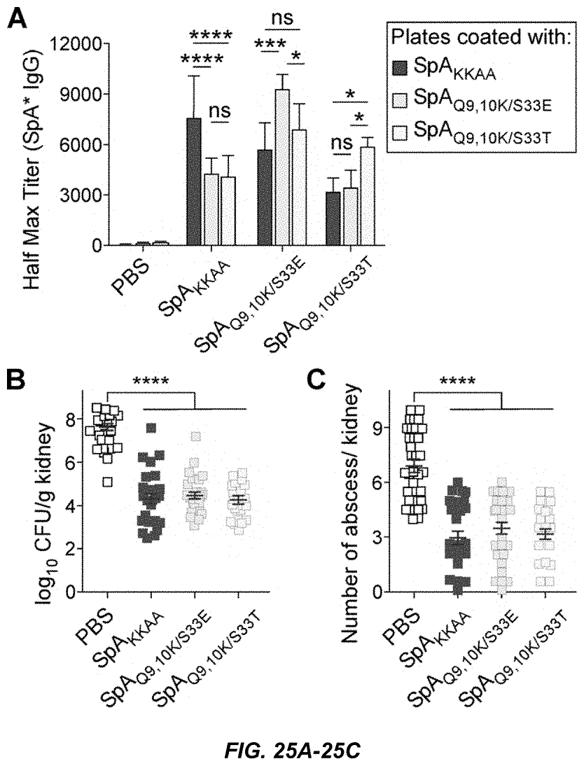

Compared to cohorts of C57BL/6 mice that were immunized with adjuvant alone (mock), immunization with SpA_{KKAA} or SpA_{Q9,10K/S33E} or SpA_{Q9,10K/S33T} generated SpA-neutralizing antibodies (FIG. 25A). As expected, SpA_{KKAA} immunization induced decolonization of S. aureus WU1 from the nasopharynx and gastrointestinal tract of C57BL/6 mice beginning 21 days following intranasal colonization (FIG. 24A, 24B, 24C). Further, in decolonized mice, SpA_{KKAA} immunization was associated with increased pathogen-specific IgG (including anti-ClfB, anti-IsdA, anti-IsdB, anti-SasG) antibodies that are associated with S. aureus decolonization [(102) and data not shown]. Similar results were observed following immunization of C57BL/6 mice with SpA_{Q9,10K/S33E}. As compared to mock control, SpA_{Q9,10K/S33E} vaccination promoted S. aureus WU1 decolonization from the nasopharynx and gastrointestinal tract of C57BL/6 mice similarly to SpA_{KKAA} vaccination (FIG. 24B, 24C). In decolonized mice, SpA_{Q9,10K/S33E} vaccination was associated with increased pathogen-specific IgG (including anti-ClfB, anti-IsdA, anti-IsdB, anti-SasG; data not shown). Compared with SpA_{KKAA} immunized animals, SpA_{Q9,10K/S33E} vaccination elicited similar levels of S. aureus decolonization, suggesting that the two vaccines exhibit similar protective efficacy in the mouse colonization model. SpA_{Q9,10K/S33T} vaccination elicited similar levels of S. aureus decolonization as SpA_{KKAA} and SpA_{Q9,10K/S33E} vaccination (data not shown). When cohorts of animals were immunized on the same days with SpA_{KKAA} or SpA_{Q9,10K/S33E} or SpA_{Q9,10K/S33T}, approximately 50% of the animals became decolonized in the nasopharynx and gastrointestinal tract while all the animals receiving adjuvant alone (mock) remained colonized (FIG. 24D, 24E). This data further demonstrate that all three candidate vaccines perform similarly in the colonization model of S. aureus.

Efficacy of SpA Vaccine Candidates in a Mouse Model for S. aureus Bloodstream Infection

Earlier work demonstrated that immunization of BALB/C mice with SpA_{KKAA} elicited SpA-specific antibodies that protected animals against intravenous MRSA USA300 LAC bloodstream challenge and the ensuing formation of abscess lesions in renal tissues (43). As compared to mock (adjuvant alone) immunized mice, immunization with SpA_{KKAA}, SpA_{Q9,10K/S33E} or SpA_{Q9,10K/S33T} elicited significantly high-titer antibodies against SpA_{KKAA}, against SpA_{Q9,10K/S33E} or against SpA_{Q9,10K/S33T} (FIG. 25A). SpA-specific antibody titers induced by SpA_{KKAA} immunization in BALB/c mice were significantly higher when analyzed by ELISA for SpA_{KK}AA than analyzed for SpA_{Q9,10K/S33E} or SpA_{Q9,10K/S33T} (SpA_{KKAA} vs. SpA_{Q9,10K/S33E}, P<0.0001; SpA_{KKAA} vs. SpA_{Q9,10K/S33T}, P<0.0001). In a similar way, SpA-specific antibody titers induced by SpA_{Q9,10K/S33E} immunization in BALB/c mice were significantly higher when analyzed by ELISA for SpA_{Q9,10K/S33E} than analyzed for SpA_{KKAA} or SpA_{Q9,10K/S33T} (SpA_{KKAA} vs. SpA_{Q9,10K/S33E}, P<0.001; SpA_{Q9,10K/S33E} VS. SpA_{Q9,10K/S33T}, P<0.05) while SpA-specific antibody titers induced by SpA_{Q9,10K/S33T} immunization in BALB/c mice were significantly higher when analyzed by ELISA for SpA_{Q9,10K/S33T} than analyzed for SpA_{KKAA} or SpA_{Q9,10K/S33E} (SpA_{KKAA} vs. SpA_{Q9,10K/S33T}, P<0.05; SpA_{Q9,10K/S33E} VS. SpA_{Q9,10K/S33T}, P<0.05) (FIG. 25A). These results suggest that some but not all of the epitopes of antibodies produced by SpA_{Q9,10K/S33E} and SpA_{Q9,10K/S33T} vaccination in BALB/c mice are different from that produced by SpA_{KKAA} vaccination and vice versa. As reported earlier (43), compared to mock-immunized mice, SpA_{KKAA} vaccination reduced the bacterial load of MRSA USA300 LAC and the number of abscess lesions in BALB/c mice (FIG. 25B; P<0.0001). SpA_{Q9,10K/S33E} and SpA_{Q9,10K/S33T} vaccination generated similar protection against MRSA USA300 LAC bloodstream infection compared to SpA_{KKAA} vaccination. Compared to mock-immunized animals, SpA_{Q9,10K/S33E} and SpA_{Q9,10K/S33T} immunization reduced the bacterial load and the number of abscess lesions in BALB/c mice (FIG. 25C; P<0.0001). Thus, SpA_{Q9,10K/S33E} and SpA_{Q9,10K/S33T} vaccination elicits similar protection against MRSA USA300 LAC bloodstream infection and associated abscess formation in mice as previously reported for the SpA_{KKAA} vaccine candidate (43).

Binding of SpA Vaccine Candidates to SpA-Neutralizing Monoclonal Antibody 3F6

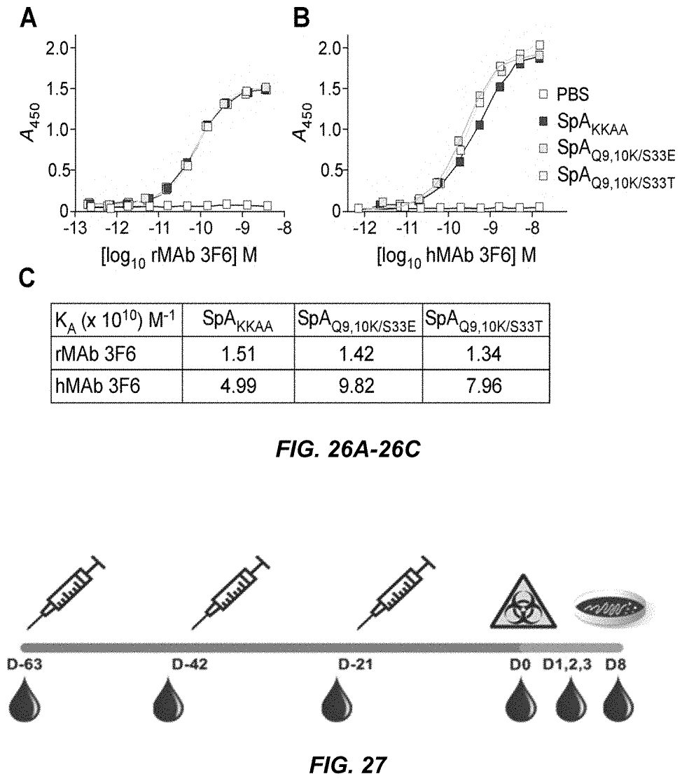

Mouse hybridoma monoclonal antibody (hMAb) 3F6 (IgG2a) was generated using splenocytes from SpA_{KKAA}-immunized BALB/c mice (84). The gene for hMAb 3F6 was sequenced and cloned into an expression vector for purification of recombinant rMAb 3F6 from HEK293 F cells (146). Both hMAb3F6 and rMAb 3F6 bind to the triple-helical fold of each of the five SpA IgBDs (E, D, A, B, and C) and neutralize their ability to bind human IgG or IgM (84, 146). Intravenous administration of hMAb3F6 or rMAb 3F6 at a dose of 5 mg/kg protects BALB/c mice against S. aureus bloodstream infection associated renal abscess formation and bacterial replication (bacterial load) (84, 146). Further, intravenous administration of rMAb 3F6 (5 mg/kg) into C57BL/6 mice induces S. aureus WU1 decolonization from the nasopharynx and gastrointestinal tract of pre-colonized animals (146). Here the inventors asked whether rMAb 3F6 binds SpA_{Q9,10K/S33E} or SpA_{Q9,10/S33T} with similar affinity as SpA_{KKAA}, the cognate antigen from which the monoclonal antibody had been derived (84). When measured via ELISA with fixed concentrations of ligands and serial dilutions of rMAb 3F6, the inventors derived affinity constants of SpA_{KKAA} (Kₐ 1.51×10¹⁰ M⁻¹) SpA_{Q9,10K/S33E} (Kₐ 1.42×10¹⁰ M⁻¹) and SpA_{Q9,10K/S33T} (Kₐ 1.34×10¹⁰ M⁻¹) for binding to SpA vaccine candidates (FIG. 26). These data suggest that the amino acid substitutions Ser³³Glu and Ser³³Thr do not affect binding of SpA-neutralizing rMAb 3F6. Further, the amino acid substitutions Ser³³Glu and Ser³³Thr do not destroy the protective SpA-epitope as defined by the binding of rMAb 3F6.

Discussion

The inventors show here that the S. aureus vaccine candidates—SpA_{KKAA}, and SpA-KR—retain significant binding to V_{H}3-idiotypic immunoglobulin when using F(ab)2 fragment of human IgG as ligand. When analyzed with human mast cells (LAD2 cells) coated with V_{H}3-IgG, SpA_{KKAA} and SpA-KR trigger V_{H}3-Ig crosslinking, as measured by the release of 3-hexosaminidase and histamine (145). In a mouse model for anaphylactic vascular hyperpermeability, the biological effects of such histamine release were measurable as Evans Blue dye extravasation at anatomical sites of V_{H}3-IgG administration in MT mice. Together these observations raise concerns about the safety of SpA vaccine candidates as potential activators of anaphylactic reactions in humans.

To address the concerns with SpA vaccines, the inventors engineered two new antigens, SpA_{Q9,10K/S33E} and SpA_{Q9,10K/S33T}, with improved safety profiles. SpA_{Q9,10K/S33E} and SpA_{Q9,10K/S33T} lack affinity for V_{H}3-idiotypic immunoglobulins, show reduced or no activity toward histamine release from V_{H}3-IgE coated human mast cells and do not promote Evans Blue dye extravasation in response to V_{H}3-IgG injection in MT mice. Immunization of BALB/c mice with SpA_{Q9,10K/S33E} and SpA_{Q9,10K/S33T} elicited similar levels of SpA-specific IgG responses as SpA_{KKAA}. When analyzed for vaccine efficacy in mouse models, vaccination with SpA_{Q9,10K/S33E} or SpA_{Q9,10K/S33T} afforded similar levels of protection against S. aureus colonization or invasive bloodstream infection as the SpA_{KKAA} vaccine (43). Further, the amino acid substitutions Ser³³Glu and Ser³³Thr do not perturb the protective IgBD epitopes that are defined by the S. aureus colonization- and invasive disease-protective monoclonal antibody 3F6 (84, 146). Based on these observations, the inventors hypothesize that the S. aureus vaccine candidates SpA_{Q9,10K/S33E} and SpA_{Q9,10K/S33T} may be suitable for development as clinical grade vaccines for clinical safety and efficacy testing against S. aureus colonization and invasive disease.

Materials and Methods

Bacterial strains and growth conditions. S. aureus strains USA300 (LAC) and WU1 were grown in tryptic soy broth (TSB) or tryptic soy agar (TSA) at 37° C. Escherichia co/i strains DH5α and BL21(DE3) were grown at 37° C. in lysogeny broth (LB) medium with 100 μg/ml ampicillin and 1 mM isopropyl β-d-1-thiogalactopyranoside (IPTG) for the production of recombinant proteins.

Construction of SpA variants. The coding sequence of SpA variants was synthesized by Integrated DNA Technologies, Inc. The sequences and plasmid pET15b+ were digested by NdeI and BamHI, respectively. Then, the two digested products were ligated and transformed into Escherichia co/i DH5u to generate the clones expressing N-terminal Hexahistidine(His6)-tagged recombinant proteins. Candidate clones were validated by DNA sequencing. The correct plasmids were transformed into E. coli BL21 (DE3) for production of SpA variant candidates.

Purification of proteins. Cultures of E. coli (2 liters) that had been grown in LB supplemented with ampicillin and IPTG to an absorbance at 600 nm (A600) of 2.0 were centrifuged (10,000×g for 10 minutes). Sedimented cells were suspended in Buffer A (50 mM Tris-HCl [pH 7.5], 150 mM NaCl), and the resulting suspensions were lysed in a French press at 14,000 lb/in² (Thermo Spectronic, Rochester, N.Y.). Unbroken cells were removed by centrifugation (5,000×g for 15 minutes), and the crude lysates subjected to ultracentrifugation (100,000×g for 1 hour at 4° C.). Soluble recombinant proteins were subjected via gravity flow to chromatography on Ni-NTA agarose (QIAGEN) with a packed volume of 1 ml preequilibrated with Buffer A. The columns were washed with 20 bed volumes of Buffer A, 20 bed volumes of Buffer A containing 10 mM imidazole and eluted with 6 ml of Buffer A containing 500 mM imidazole. Aliquots of the eluted fractions were mixed with equal volumes of sample buffer and separated on 15% sodium dodecyl sulfate-polyacrylamide gel electrophoresis (SDS-PAGE) gels. Recombinant proteins were dialyzed against phosphate-buffered saline (PBS) and their concentrations determined with the bicinchoninic acid assay (Pierce). For immunization studies in animals and for incubation with cell lines, recombinant protein preparations were subjected to the Endotoxin Removal Spin Columns (Pierce) to eliminate contaminating LPS. Sample purity was tested with ToxinSensor™ Chromogenic LAL Endotoxin Assay Kit (Genscript).

Purification of antibodies. To purify VH3 IgG, human plasma (20 ml) prepared using whole human blood was subjected to affinity chromatography over Protein G Resin (Genscript) to remove human IgM, IgD and IgA. Immunoglobulins eluted from Protein G Resin were subjected to a second affinity chromatography, SpA_{K}-coupled resin to enrich for VH3 IgG [SpA_{K} cannot bind the Fcγ domain of IgG (48)]. Protein G Resin and SpA_{K}-coupled resin were washed with 20-column volumes of PBS and bound proteins eluted with 0.1M glycine pH 3.0, neutralized with 1 M Tris-HCl, pH 8.5, and dialyzed against PBS overnight. For VH3 IgE purification, the human cell line HEK 293F was used for transient expression of pVITRO1-Transtuzumab-IgE-κ. Cells were grown in DMEM/HIGH GLUCOSE medium with 10% FCS, 2 mM glutamine, penicillin (5,000 U/ml) and streptomycin (100 μg/ml). Cells transfected with pVITRO1-Transtuzumab-IgE-κusing PEI were incubated at 37° C. in a 5% CO₂ atmosphere. For the stable expression of IgE, cells were cultured in Freestyle 293 medium, for 7 days, and harvested at 12000×g for 20 minutes. The supernatant was purified over 2 ml Protein L Resin (Genscript). The resin was washed with 20-column volumes of PBS and bound VH3 IgE eluted with 0.1M glycine pH 3.0, neutralized with 1 M Tris-HCl, pH 8.5, and dialyzed against PBS overnight.

Surface Plasmon Resonance (SPR). SPR experiments shown in Tables 5, 6, 7 and 9 were performed on ProteOn™ XPR36 with ProteOn HTG chip. The running buffer was PBS with 0.05% Tween-20. The sensor-chip surfaces were activated with 2 mM nickel sulfate and regenerated with 300 mM EDTA, respectively. 500 nM of test articles (SpA wild type or variants) were immobilized at a flow rate of 25 μl/min. To measure interactions with wild type SpA, ligands (purified immunoglobulins) were used at concentrations of 500, 400, 300, 200 and 100 nM. To measure interactions with SpA variants, ligands were used at concentrations of 4, 3, 2, 1 and 0.5 μM. The association and dissociation rates were measured at a continuous flow rate of 30 μl/min and analyzed using the two-state reaction model. Association constants were determined from three independent experiments.

Bio-layer Interferometry (BLI). The BLI experiment shown in Table 9 was performed using BLItz Bio-Layer Interferometer. Test candidates (25-50 nM) were immobilized onto Ni-NTA sensor for 120 seconds. The sensor was equilibrated with PBS for 80 seconds, dipped in solutions containing ligand at concentrations of 20, 15, 10, and 0 μM for 120 seconds (association phase) followed by 120 seconds in PBS (dissociation phase). The data was acquired using BLI Data acquisition software 9.0 (ForteBIO) and analyzed using the Data Analysis software 9.0.0.14 (ForteBIO). Reported association values were calculated from curves fitted model.

Enzyme-Linked Immunosorbent Assay (ELISA). Microtiter plates (NUNC MaxiSorp) were coated with purified antigens at 1 μg/ml (to measure antibody titers in test sera) or at 0.5 μg/ml (to measure interaction with 3F6 antibodies) in 0.1 M carbonate buffer (pH 9.5) at 4° C. overnight. Wells were blocked and incubated with test serum or 3F6 antibodies prior to incubation with horseradish peroxidase (HRP)-conjugated mouse or human IgG (1 μg/ml, Jackson ImmunoResearch). All plates were incubated with mouse HRP-conjugated secondary antibody specific (Fisher Scientific) and developed using OptEIA reagent (BD Biosciences). Half max titers were calculated with the GraphPad Prism software. The association constant was calculated from nonlinear regression (curve fit) model in the GraphPad Prism software. All experiments were performed in triplicate to calculate averages and standard error of the mean, and repeated for reproducibility.

Anaphylactic response in pMT mice. Mice with the pMT mutation were purchased from the Jackson Laboratory and bred at the University of Chicago. Cohorts of 5 six-week old female mice per group were sensitized by intradermal injection in the ear with VH3 IgG (2 μg in 20 μl of PBS) and 24 hours later, injected intravenously under anesthesia with ketamine-xylazine (100 mg-20 mg/kg) into the periorbital venous sinus of the right eye, with either PBS, SpA or its variants (200 μg in 100 μl PBS). Following 5 minutes stimulation with test article, animals were injected intravenously into the periorbital venous sinus of the left eye with 100 μl of 2% Evans blue. Animals were killed, ears dissected, dried, and extracted in formamide for 24 hours at 65° C. Evans blue extravasation in ear tissues (vascular permeability) was quantified by measuring absorbance at 620 nm.

Human basophil activation experiments. Blood (10 ml) was obtained from healthy donors and immediately mixed with 1 ml EDTA 0.1 M, pH7.5. SpA wild-type or vaccine candidate variants (1 μg) or PBS were added to 1-ml EDTA blood aliquots and samples were incubated for 1 hour at 37° C. with rotation. Sample aliquots were treated with RBC lysis buffer (Biolegend), centrifuged (350×g) and supernatants discarded. Cells in pellets were washed in cold PBS and re-suspended in PBS with 5% FBS for staining with anti-CD123-FITC, anti-HLA-DA-PerCP, anti-CD63-PE, and anti-CD203c-APC (Biolegend) in the dark at room temperature for 10 min. All stained samples were analyzed using BD LSRII 3-8 (BD Biosciences). Total basophil counts were obtained by gating from SSClow/CD203c+/CD123+/HLA-DR− cells and activated basophils were selected from the CD63+CD203c+ pool. Experiments were performed in triplicate and repeated at least three times using different healthy donors.

Mast cell degranulation. Human mast cells (LAD2) [kindly provided by Dr. Kirshenbaum from NIAID] were sensitized by incubating 2×10⁵ cells with 100 ng VH3 IgE, overnight at 37° C. in a 5% CO₂ atmosphere. Cells were harvested and washed twice with HEPES buffer containing 0.04% bovine serum albumin (BSA) to remove free IgE. Cells were suspended in the same buffer at the concentration of 2×10⁵ cells/ml, and stimulated with SpA or test articles for 30 min before assaying for β-hexosaminidase and histamine release. Cells were sedimented and the spent medium was transferred to a fresh tube while cells in the pellet were lysed with 0.1% Triton X-100. β-hexosaminidase activity in the spent medium and the Triton X-100-lyzed cells, was measured by adding the colorimetric substrate pNAG (p-nitrophenyl-N-acetyl-β-D-glucosaminide obtained from Sigma; final concentration 3.5 mg/ml at pH 4.5) for 90 min. The reaction was quenched by addition of 0.4 M glycine pH 10.7 and absorbance at λ=405 nm recorded. The results were expressed as the percentage of O-hexosaminidase released in the spent medium over total (spent medium+Triton X-100 lyzed cells). Experiments were performed in triplicates and repeated at least three times. Histamine was measured using an Enzyme Immunoassay (SpiBio Bertin Pharma). Briefly, wells of a microtiter plate were coated with mouse anti-histamine antibody and incubated for 24 hours with tracer (acetylcholinesterase linked to histamine) mixed with an experimental extract. Plates were washed, and Ellman's Reagent (acetylcholinesterase substrate) was added to the wells. Product formation was detected by recording absorbance at 412 nm. Absorbance at 412 nm is proportional to the amount of tracer bound to the well and is inversely proportional to the amount of histamine present in the experimental extract. All samples were performed in duplicate.

Active immunization of mice. Animals BALB/c or C57BL/6J (3 week-old, female mice, 15 animals per group) were immunized with PBS, or 50 μg purified endotoxin-free protein SpA_{KKAA} or SpA_{Q9,10K/S33E} or SpA_{Q9,10K/S33T} emulsified in 5:2:3 of antigen:CFA:IFA and boosted with 50 μg proteins emulsified in 1:1 of antigen:IFA 11 days following the first immunization. On day 20, mice were bled and serum was harvested to evaluate antibody titers to vaccine candidates by ELISA. On day 21, mice were either inoculated for nasopharyngeal colonization or infected by the intravenous injection of bacteria.

Mouse nasopharyngeal colonization. Overnight cultures of S. aureus strain WU1 were diluted 1:100 in fresh TSB and grown for 2 h at 37° C. as described (102). The cells were centrifuged, washed, and suspended in PBS. 10 immunized female C57BL/6J mice per group (Jackson Laboratory) were anesthetized by intraperitoneal injection with ketamine-xylazine (100 mg-20 mg/kg), and 1×10⁸ CFU of S. aureus (in a 10-1 volume) was pipetted into the right nostril of each mouse. In weekly intervals following inoculation, the oropharynx of the mice was swabbed and stool samples were collected and homogenized in PBS. Swab samples and homogenates of stool samples were spread on mannitol salt agar (MSA) for bacterial enumeration. At the end of the experiment, the mice were bled via periorbital vein puncture to obtain sera for antibody response analyses using the staphylococcal antigen matrix as described (43). Briefly, nitrocellulose membranes were blotted with 2 μg affinity-purified staphylococcal antigens. The membranes were blocked with 5% degranulated milk and incubated with diluted mouse sera (1:10,000 dilution) and IRDye 680-conjugated goat anti-mouse IgG (Li-Cor). Signal intensities were quantified using the Odyssey infrared imaging system (Li-Cor). All animal experiments were performed in duplicate. Two-way analysis of variance (ANOVA) with Sidak multiple-comparison tests (GraphPad Software) was performed to analyze the statistical significance of nasopharyngeal and stool colonization, ELISA, and antigen matrix data.

Mouse renal abscess model. Overnight cultures of S. aureus USA300 (LAC) were diluted 1:100 into fresh TSB and grown for 2 h at 37° C. Staphylococci were sedimented, washed, and suspended in PBS. Inocula were quantified by spreading sample aliquots on TSA and enumerating the colonies that formed upon incubation. Groups of 15 BALB/c mice immunized with endotoxin-free protein SpA_{KKAA} or SpA_{Q9,10K/S33E} or SpA_{Q9,10K/S33T} prepared in PBS or mock immunized (PBS control) were anesthetized and inoculated with 5×10⁶ CFU of S. aureus USA300 (LAC) into the periorbital venous sinus of the right eye. On day 15 following challenge, mice were killed by CO₂ inhalation. Both kidneys were removed, and the staphylococcal load in one organ was analyzed by homogenizing renal tissue with PBS, 0.1% Triton X-100. Serial dilutions of homogenate were spread on TSA and incubated for colony formation. The remaining organ was examined by histopathology. Briefly, kidneys were fixed in 10% formalin for 24 h at room temperature. Tissues were embedded in paraffin, thin sectioned, stained with hematoxylin-eosin, and inspected by light microscopy to enumerate abscess lesions. All animal experiments were performed in duplicate and statistical analysis were calculated with t-tests (and nonparametric tests) of Graphpad Prism.

Ethics statement. Experiments with blood from human volunteers were performed with a protocol reviewed, approved, and supervised by the University of Chicago's Institutional Review Board (IRB). All mouse experiments were performed in accordance with the institutional guidelines following experimental protocol review and approval by the Institutional Biosafety Committee (IBC) and the Institutional Animal Care and Use Committee (IACUC) at the University of Chicago.

Statistical analyses. For FIGS. 22, 23, 25, and Tables 5-10, one-way ANOVA with post-test (Bonferroni's or Dunnett's Multiple Comparison Test) was used to derive statistical significance between the means of multiple groups. For FIG. 24, two-way analysis of variance (ANOVA) with Sidak multiple-comparison tests (GraphPad Software) was performed to analyze the statistical significance of mouse colonization and the staphylococcal antigen matrix data. All data were analyzed by Prism (GraphPad Software, Inc.), and P values less than 0.05 were deemed significant.

Tables

TABLE 5
Affinity measurements with wild-type SpA, SpA_{KKAA} and
SpA_{Q9,10K/G29X} vaccine candidates and human IgG^{#}.

| SpA_{Q9,10K/G29X}^{a} | K_{A} (×10⁵ M⁻¹) ^{b} | SD (×10⁵) ^{c} | P value^{d} |
|---|---|---|---|
| SpA_{Q9,10K/G29H} | 1.435 | 0.2799 | * |
| SpA_{Q9,10K/G29C} | 1.743 | 0.8619 | * |
| SpA_{Q9,10K/G29T} | 1.982 | 0.9146 | ns |
| SpA_{Q9,10K/G29Q} | 2.057 | 0.9600 | * |
| SpA_{Q9,10K/G29L} | 3.146 | 1.3860 | ns |
| SpA_{Q9,10K/G29E} | 3.182 | 1.5300 | ns |
| SpA_{Q9,10K/G29P} | 3.396 | 1.4410 | ns |
| SpA_{Q9,10K/G29F} | 3.460 | 1.5860 | ns |
| SpA_{Q9,10K/G29M} | 3.893 | 0.7868 | ns |
| SpA_{Q9,10K/G29V} | 4.350 | 1.0830 | ns |
| SpA_{Q9,10K/G29W} | 4.508 | 0.7448 | ns |
| SpA_{Q9,10K/G29D} | 5.478 | 1.0150 | ns |
| SpA_{Q9,10K/G29R} | 6.056 | 0.9814 | ns |
| SpA_{Q9,10K/G29N} | 6.231 | 0.7696 | ns |
| SpA_{Q9,10K/G29Y} | 8.367 | 3.326 | ns |
| SpA_{Q9,10K/G29S} | 9.398 | 4.298 | *** |
| SpA_{Q9,10K/G29K} | 9.738 | 2.345 | ** |
| SpA_{Q9,10K/G29I} | 10.070 | 4.398 | ** |
| SpA_{Q9,10K/G29A} | 11.310 | 3.119 | *** |
| SpA_{KKAA} | 5.022 | 2.150 | |
| SpA | 1081 | 16.34 | |

^{a}Test articles were immobilized on Bio-Rad ProteOn HTG Chip and subjected to Surface Plasmon Resonance measurements with increasing concentrations of human IgG and flowed over each channel of the chip. Data were analyzed from three independent experimental determinations.
^{b} Data were used to derive the association constant (K_{A}) for each test article.
^{c} Data were used to derive the Standard Deviation (SD) for each test article.
^{d}Data were analyzed with One-way ANOVA with Dunnett's Multiple Comparison Test between each test article and SpA_{KKAA}.
Symbols: ns, not significant;
* P < 0.05;
** P < 0.01;
*** P < 0.001;
**** P < 0.0001.

TABLE 6
Affinity measurements with wild-type SpA, SpA_{KKAA} and
SpA_{Q9,10K/S33X} vaccine candidates and human IgG^{#}.

| SpA_{Q9,10K/S33X}^{a} | K_{A} (×10⁵ M⁻¹) ^{b} | SD (×10⁵) ^{c} | P value^{d} |
|---|---|---|---|
| SpA_{Q9,10K/S33E} | 0.496 | 0.0439 | ** |
| SpA_{Q9,10K/S33T} | 0.386 | 0.1218 | *** |
| SpA_{Q9,10K/S33Y} | 1.571 | 0.7497 | ns |
| SpA_{Q9,10K/S33I} | 1.840 | 1.1290 | * |
| SpA_{Q9,10K/S33L} | 2.051 | 0.7592 | ns |
| SpA_{Q9,10K/S33W} | 2.356 | 0.6373 | ns |
| SpA_{Q9,10K/S33V} | 2.471 | 1.2060 | ns |
| SpA_{Q9,10K/S33H} | 2.784 | 0.6087 | ns |
| SpA_{Q9,10K/S33N} | 3.066 | 1.0100 | ns |
| SpA_{Q9,10K/S33M} | 3.177 | 1.3750 | ns |
| SpA_{Q9,10K/S33R} | 3.463 | 1.7950 | ns |
| SpA_{Q9,10K/S33D} | 3.824 | 1.7100 | ns |
| SpA_{Q9,10K/S33F} | 3.902 | 1.8040 | ns |
| SpA_{Q9,10K/S33Q} | 4.068 | 2.8350 | ns |
| SpA_{Q9,10K/S33P} | 4.218 | 2.2560 | ns |
| SpA_{Q9,10K/S33C} | 4.577 | 0.6927 | ns |
| SpA_{Q9,10K/S33K} | 5.124 | 2.1810 | ns |
| SpA_{Q9,10K/S33A} | 10.540 | 5.0520 | *** |
| SpA_{Q9,10K/S33G} | 11.180 | 5.2040 | *** |
| SpA_{KKAA} | 5.022 | 0.0439 | |
| SpA | 1081⁻¹ | 16.34 | |

^{a}Test articles were immobilized on Bio-Rad ProteOn HTG Chip and subjected to Surface Plasmon Resonance measurements with increasing concentrations of human IgG and flowed over each channel of the chip. Data were analyzed from three independent experimental determinations.
^{b} Data were used to derive the association constant (K_{A}) for each test article.
^{c} Data were used to derive the Standard Deviation (SD) for each test article.
^{d}Data were analyzed with One-way ANOVA with Dunnett's Multiple Comparison Test between each test article and SpA_{KKAA}.
Symbols: ns, not significant;
* P < 0.05;
** P < 0.01;
*** P < 0.001;
**** P < 0.0001.

TABLE 7
The association constant for binding to human IgG of SpA variants
Q9,10K/S33X or Q9,10K/G29X in combination with other amino acid substitutions#

| | | | | Parent SpA variant | | | |
|---|---|---|---|---|---|---|---|
| Parent SpA | K_{A} | | P | with additional | K_{A} | SD | P |
| variant^{a} | (×10⁵ M⁻¹)^{b} | SD^{c} | value^{d} | substitutions^{a} | (×10⁵ M⁻¹)^{b} | (×10⁵ M⁻¹)^{c} | value^{e} |
| SpA_{Q9,10K/S33E} | 0.496 | 0.044 | * | SpA_{Q9,10K/S33E/D36,37A} | 0.568 | 0.1185 | ns |
| | | | | SpA_{Q9,10K/S33E/G29A} | 1.265 | 0.6947 | ns |
| | | | | SpA_{Q9,10K/S33E/D36,37A/G29A} | 1.892 | 0.6793 | ns |
| | | | | SpA_{Q9,10K/S33E/G29F} | 1.575 | 0.4060 | ns |
| | | | | SpA_{Q9,10K/S33E/D36,37A/G29F} | 14.850 | 13.480 | *** |
| | | | | SpA_{Q9,10K/S33E/G29R} | 4.840 | 1.1960 | ns |
| | | | | SpA_{Q9,10K/S33E/D36,37A/G29R} | 10.240 | 5.2600 | * |
| SpA_{Q9,10K/S33Q} | 4.068 | 2.835 | ns | SpA_{Q9,10K/S33Q/D36,37A} | 3.930 | 1.9290 | ns |
| | | | | SpA_{Q9,10K/S33Q/G29A} | 2.563 | 1.3670 | ns |
| | | | | SpA_{Q9,10K/S33Q/D36,37A/G29A} | 4.893 | 3.8360 | ns |
| | | | | SpA_{Q9,10K/S33Q/G29F} | 1.275 | 0.7355 | ns |
| | | | | SpA_{Q9,10K/S33Q/D36,37A/G29F} | 12.470 | 8.8810 | * |
| | | | | SpA_{Q9,10K/S33Q/G29R} | 2.333 | 0.4245 | ns |
| | | | | SpA_{Q9,10K/S33Q/D36,37A/G29R} | 6.378 | 4.6820 | ns |
| SpA_{Q9,10K/S33F} | 3.902 | 1.804 | ns | SpA_{Q9,10K/S33F/D36,37A} | 3.634 | 2.6420 | ns |
| | | | | SpA_{Q9,10K/S33F/G29A} | 1.190 | 0.4299 | ns |
| | | | | SpA_{Q9,10K/S33F/D36,37A/G29A} | insoluble | | |
| | | | | SpA_{Q9,10K/S33F/G29F} | 2.440 | 0.7657 | ns |
| | | | | SpA_{Q9,10K/S33F/D36,37A/G29F} | insoluble | | |
| | | | | SpA_{Q9,10K/S33F/G29R} | 1.903 | 0.8693 | ns |
| | | | | SpA_{Q9,10K/S33F/D36,37A/G29R} | 9.056 | 4.9730 | * |
| SpA_{Q9,10K/S33K} | 5.124 | 2.181 | ns | SpA_{Q9,10K/S33K/D36,37A} | 8.048 | 4.1050 | ns |
| SpA_{Q9,10K/S33A} | 10.540 | 5.052 | *** | SpA_{Q9,10K/S33A/D36,37A} | 18.830 | 18.320 | ns |
| SpA_{Q9,10K/G29F} | 3.460 | 1.586 | ns | SpA_{Q9,10K/G29F/D36,37A} | 3.723 | 1.5100 | ns |
| SpA_{Q9,10K/G29R} | 6.056 | 0.981 | ns | SpA_{Q9,10K/G29R/D36,37A} | 6.808 | 3.6840 | ns |
| SpA_{Q9,10K/G29A} | 11.310 | 3.119 | ** | SpA_{Q9,10K/G29A/D36,37A} | 1.78 | 0.5098 | *** |
| SpA-KR | 5.464 | 0.767 | ns | | | | |
| SpA_{RRVV} | 5.609 | 2.355 | ns | | | | |
| SpA_{KKAA} | 5.022 | 2.150 | — | | | | |

^{a}Test articles were immobilized on Bio-Rad ProteOn HTG Chip and subjected to Surface Plasmon Resonance measurements with increasing concentrations of human IgG and flowed over each Chip. Data were analyzed from three independent experimental determinations.
^{b}Data were used to derive the association constant (K_{A}) for each test article.
^{c}Data were used to derive the standard deviation (SD) for each test article.
^{d, e}Data were analyzed with One-way ANOVA with Dunnett’s Multiple Comparison Test
between test article and SpA_{KKAA}^{d} and between test article (column 5) and parent vaccine (column 1)^{e}.
Symbols:
ns, not significant:
*, P < 0.05;
**, P < 0.01;
***, P < 0.001;
****, P < 0.0001.

TABLE 8
The association constant for binding of each combination
mutation to F(ab)2 fragment of human IgG

| SpA variant^{a} | K_{A} ( M⁻¹)^{b} | SD^{c} | P value^{d} |
|---|---|---|---|
| SpA | 1.44 × 10⁷ | 8.193 × 10⁶ | — |
| SpA_{KKAA} | 8.27 × 10⁴ | 2.76 × 10⁴ | — |
| SpA-KR | 6.42 × 10⁴ | 3.80 × 10⁴ | ns |
| SpA_{Q9,10K/S33E} | 41.24 | 5.386 | *** |
| SpA_{Q9,10K/S33T} | 43.55 | 5.737 | *** |

^{a}Test articles were immobilized on Bio-Rad ProteOn HTGsensor and subjected to Surface Plasmon Resonance (SPR) with increasing concentrations of F(ab)2 fragment of human IgG. Data were analyzed from three independent experimental determinations.
^{b}Data were used to derive the association constant (K_{A}) for each test article.
^{c}Data were used to derive Standard Deviation (SD) for each test article.
^{d}Data were analyzed with One-way ANOVA with Dunnett's Multiple Comparison Test between test article and SpA_{KKAA}.
Symbols: ns, not significant;
* P < 0.05;
** P < 0.01;
*** P < 0.001;
**** P < 0.0001.

TABLE 9
The association constant for binding of each combination
mutation to Fcγ fragment of human IgG

| SpA variant^{a} | K_{A} (M⁻¹)^{b} | SD^{c} | P value^{d} |
|---|---|---|---|
| SpA | 5.17 × 10⁷ | 8.995 × 10⁶ | — |
| SpA_{KKAA} | 32.91 | 16.291 | — |
| SpA_{Q9,10K/S33E} | 32.68 | 16.414 | ns |
| SpA_{Q9,10K/S33T} | 39.91 | 17.081 | ns |
| SpA-KR | 39.12 | 13.348 | ns |

^{a}Test articles were immobilized on Ni-NTA sensor and subjected to Bio-Layer Interferometer (BLI) with increasing concentrations of Fc fragment of human IgG. Data were analyzed from three independent experimental determinations.
^{b}Data were used to derive the association constant (K_{A}) for each test article.
^{c}Data were used to derive the Standard Deviation (SD) for each test article.
^{d}Data were analyzed with One-way ANOVA with Dunnett's Multiple Comparison Test between test article and SpA_{KKAA}.
Symbols: ns, not significant;
* P < 0.05;
** P < 0.01;
*** P < 0.001;
**** P < 0.0001.

TABLE 10
Activation of human basophils by SpA and vaccine candidate variants

| SpA variant/PBS^{a} | PBS | SpA | SpA_{KKAA} | SpA_{Q9,10K/S33E} | SpA_{Q9,10K/S33T} | SpA-KR | SpA_{RRVV} |
|---|---|---|---|---|---|---|---|
| % activated basophils | 4.39 ± 0.884 | 32.05 ± 0.919 | 10.66 ± 1.612 | 5.38 ± 0.318 | 4.57 ± 0.877 | 8.15 ± 1.018 | 10.16 ± 0.905 |
| P value vs. PBS^{b} | — | **** | ** | ns | ns | ns | ** |
| P value vs. SpA_{KKAA}^{c} | | **** | — | * | ** | ns | ns |

^{a}Test articles were incubated with human basophils and data displayed as the percentage of activated basophils of the total basophil population (100%).
^{b,c}One-way ANOVA with Bonferroni's Multiple Comparison Test was performed for statistical analysis that compare test article and PBS^{b} or test article and SpA_{KKAA}^{c}.
Symbols: ns, not significant;
* P < 0.05;
** P < 0.01;
*** P < 0.001;
**** P < 0.0001.

### Example 4: Immune Responses Due to Immunogenic Compositions Comprising SpA Variant Polypeptide and LukAB Dimer Polypeptide Utilizing the Surgical-Wound Minipig Infection Model

The aim of the experiment is to evaluate whether a combination of a SpA variant antigen and a mutant LukAB dimer provides protection in a S. aureus surgical-wound infection model in Gottingen minipigs. The Spa variant antigen (Spa*) that was tested had an amino acid sequence of SEQ ID NO:60. The mutant LukAB dimer that was tested comprises a mutant LukA polypeptide having a deletion of the amino acid residues corresponding to positions 315-324 of SEQ ID NO:16; and a LukB polypeptide comprising the amino acid sequence of SEQ ID NO:53.

The minipig model is used to evaluate both immunogenicity (with respect to generation of antigen-specific IgG) and efficacy of the vaccine candidates. Minipigs have been widely used in infectious disease research as their immune system and organ and skin structure are largely similar to those of humans (1-5). In the model, after infection of a wound with S. aureus bacteria, a local infection develops throughout the layers of muscle and skin at the surgical site, and dissemination to other internal organs is also seen, and the progression of the disease is highly similar to that in humans.

LukAB shows a similar toxicity to minipig polymorphonuclear neutrophils (PMNs) as seen against human PMNs, in contrast to the highly-reduced toxicity against mouse or rabbit PMNs due to species-specificity of the target of the toxin. Furthermore, due to frequent carriage of Staphylococcal species by pigs, minipigs often have high levels of pre-existing antibodies to Staphylococcal antigens (including LukAB and other S. aureus proteins), similar to adult humans and in contrast to most laboratory rodents. This model is therefore likely to be a more reliable indicator of potential vaccine protection in humans, particularly for vaccines containing LukAB and Spa variant, than previously-available rodent models.

In Vivo Experiment

Male Gottingen Minipigs (3 pigs per group) were immunized intramuscularly on 3 separate occasions at 3-week intervals according to the schedule shown in FIG. 27, and the following groups:
LukAB (100 μg)+adjuvant;
SpA variant (50 μg)+adjuvant;
LukAB (100 μg)+SpA variant (50 μg)+adjuvant;
Adjuvant alone.

The vaccines were formulated with AS01b adjuvant in order to give half a human dose per animal (25 μg of MPL, and 25 μg of QS-21 per animal).

Following vaccination, the pigs were challenged with a clinically-relevant S. aureus strain. At day +8 post-infection, pigs were euthanized and the bacterial burden at the surgical site (skin and muscle) and internal organs was determined.

Blood samples were taken prior to the start of the study and at regular intervals during the vaccination and infection periods, as shown in FIG. 27. Blood and serum analysis were performed to evaluate serum immunoglobulin quantity and function as well as concentrations of biomarkers of infection and inflammation. Body temperatures were also monitored routinely as a readout of vaccine reactogenicity and infection.

The primary endpoint of the study was the reduction in bacterial burden (CFU) at surgical site/organs in animals vaccinated with the LukAB+SpA variant. Vaccinations solely with the LukAB, SpA, or adjuvant only are used as controls.

Results:

Two separate in vivo experiments in minipigs were performed. The immunization schedule and immunization groups were identical in both studies, as shown in FIG. 27 and described in the section “in vivo experiment,” respectively. In one study, an S. aureus strain belonging to Clonal Complex (CC) 389 was used as infecting strain. In the second study, an S. aureus strain belonging to CC 8 (USA300) was used as infecting strain. The characteristics of the strains are described in Table 11.

TABLE 11
Characteristics of S. aureus strains used as infecting strains in the minipig
surgical site infection model

| | | | | SpA | SpA | LukA/B | SpA | |
|---|---|---|---|---|---|---|---|---|
| Strain | Clonal | Genomic | Capsule | Super- | Cell | (proteo- | (proteo- | |
| name | Complex | Charact. | Expression | natant | Wall | mics) | mics) | Toxicity |
| USA300 | 8 | CP5⁻ | − | High | High | Moderate | High | Toxic |
| | | MRSA | | | | | | |
| | | LukAB⁺ | | | | | | |
| | | LukED⁺ | | | | | | |
| | | PVL⁺ Hlg⁺ | | | | | | |
| | | Hla⁺ Spa⁺ | | | | | | |
| CC398 | 398 | CP5⁺ | + | Moderate | Moderate | High | Moderate/Low | Toxic |
| | | MSSA | | | | | | |
| | | LukAB⁺ | | | | | | |
| | | LukED⁻ | | | | | | |
| | | PVL⁻ Hlg⁺ | | | | | | |
| | | Hla⁺ Spa⁺ | | | | | | |

Antibody Responses Induced Against LukAB and SpA*

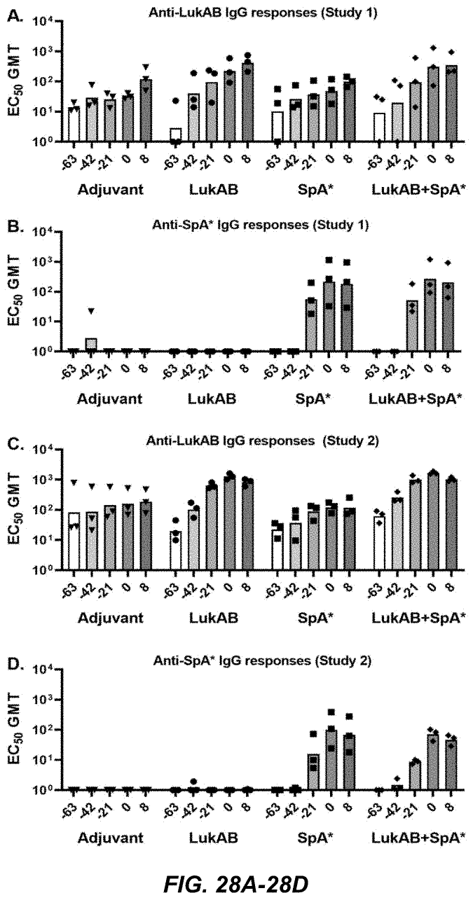

The groups of minipigs mentioned above were immunized on three occasions, three weeks apart with LukAB (100 μg)+adjuvant, or SpA* (50 μg)+adjuvant, or the combination of LukAB (100 μg) and SpA* (50 μg)+adjuvant. A control group of animals was immunized with the adjuvant only. Animals were challenged with S. aureus three weeks after the third immunization. Blood samples were taken before each immunization, before challenge, and at regular intervals until 8 days after challenge (FIG. 27) and analyzed for antibody responses against LukAB and SpA* by ELISA. Due to the short intervals of sampling after challenge, only results on day 8 after challenge are shown in FIG. 28. In the animals immunized with the adjuvant only, low levels of anti-LukAB IgG antibodies were measurable, indicating the presence of pre-existing antibodies to LukAB (FIGS. 28 A and C). Immunization with SpA*+adjuvant resulted in similar geometric mean titers of anti-LukAB antibodies as in the adjuvant control group (FIG. 28). Immunization of minipigs with LukAB+adjuvant or LukAB+SpA*+adjuvant resulted in higher geometric mean anti-LukAB IgG titers compared to the adjuvant control group in both studies after three (day 0) immunizations (FIG. 28, Study 1, Geometric mean (GeoMean) titers at day 0: LukAB+adjuvant: 222; LukAB+SpA*+adjuvant: 308; adjuvant group: 32, P>0.05; Study 2, GeoMean titers at day 0: LukAB+adjuvant: 1271; LukAB+SpA*+adjuvant: 1671; adjuvant group=159; P=0.0181 and P=0.0103, respectively vs adjuvant group)). These results indicate that immunization with the LukAB containing vaccines induced the generation of LukAB specific IgG antibodies in minipigs. The levels of anti-LukAB antibodies were similar in animals vaccinated with LukAB+adjuvant or LukAB+SpA*+adjuvant. This indicates that addition of SpA* did not interfere with the responses against LukAB.

Minipigs immunized with the adjuvant only or LukAB+adjuvant had no measurable antibodies against SpA* at any time point. SpA*+adjuvant or SpA*+LukAB+adjuvant induced a significant increase of anti-SpA* IgG after three immunizations (Study 1: GeoMean IgG in SpA*+adjuvant group: 217 and SpA*+LukAB+adjuvant group 268; Study 2: GeoMean IgG in SpA*+adjuvant group: 100 and SpA*+LukAB+adjuvant group: 71). These results indicate an induction of SpA* specific antibodies by the SpA*+adjuvant and LukAB+SpA*+adjuvant vaccines. Levels of anti-SpA* antibodies were similar in the animals that were immunized with SpA*+adjuvant or LukAB+SpA*+adjuvant. This indicates no interference on the response to SpA* by addition of LukAB.

Neutralization of the Cytotoxic Activity of LukAB Toxin

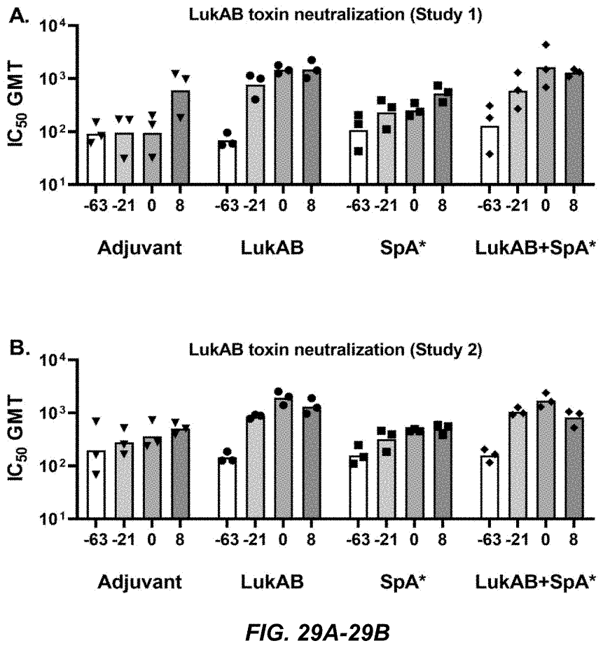

LukAB is a toxin that binds to receptors on neutrophils where it forms pores in the membrane and results in lysis of the cell. To assess the functionality of antibodies induced by the test vaccines, the ability of the sera from the vaccinated minipigs to inhibit LukAB toxin induced lysis of THP-1 cells was measured. The wild type LukAB toxin in the assay was from the clonal complex CC8, which is homologous to the LukAB clonal complex used in the vaccine (LukAB CC8 delta 10C). Background IC₅₀ titers were detectable in minipigs vaccinated with the adjuvant only (Study 1: Day 0 GeoMean IC₅₀=95; Study 2: Day 0 GeoMean IC₅₀=363). In animals vaccinated with LukAB+adjuvant or LukAB+SpA*+adjuvant, significantly higher GeoMean IC₅₀ titers were measured after three imunizations (GeoMean IC₅₀ titers after three immunizations before challenge: Study 1: LukAB+adjuvant: 1475; LukAB+SpA*+adjuvant: 1643 P>0.012 and P=0.01 vs adjuvant group; Study 2: LukAB+adjuvant: 1931; LukAB+SpA*+adjuvant: 1717; P=0.0022 and 0.0032, respectively vs adjuvant group). These results, shown in FIGS. 29A and 29B, indicate that LukAB in the vaccine induces functional antibodies that block the cytotoxic activity of the LukAB toxin.

Efficacy in the Minipig Surgical Wound Infection Model

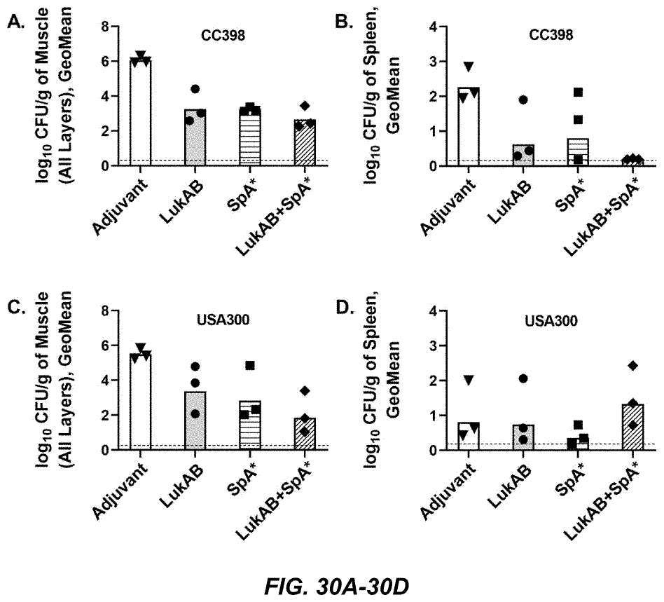

To test efficacy of the vaccines we determined the number of colony forming units (cfu) in the muscle and spleen after three immunizations and challenge with S. aureus. Two different challenge strains were used in the two studies, one belonged to clonal complex CC398, the second was a USA300 strain from clonal complex CC8. Immunization with the adjuvant only resulted in high levels of cfu in the total muscle after challenge with the CC398 strain (GeoMean log₁₀ cfu/g muscle=6.05). Immunization with LukAB+adjuvant (GeoMean log₁₀ cfu/g muscle=3.25, P=0.0036), SpA*+adjuvant (GeoMean log₁₀ cfu/g muscle=3.22, P=0.003) or the combination of LukAB+SpA*+adjuvant (GeoMean log₁₀ cfu/g muscle=2.66, P=0.0012) resulted in a significant decrease of cfu in the muscle compared to the adjuvant group (FIG. 30A). Also in the spleen, high levels of cfu were observed in the control group immunized with the adjuvant only (GeoMean log₁₀ cfu/g spleen=2.26). Immunization with LukAB+adjuvant and SpA*+adjuvant resulted in a decrease of cfu in the spleen (GeoMean log₁₀ cfu/g spleen=0.29 and 0.78, respectively, P>0.05). A significant decrease of cfu to the lower limit of quantification was detected in the spleen when animals were immunized with the combination of LukAB+SpA*+adjuvant (GeoMean log₁₀ cfu/g spleen=0.2, P=0.0424), (FIG. 30B). The results show that the test vaccine is efficacious in the minipig surgical site infection model. The vaccines also reduced the spread of the bacteria to organs like the spleen, where the combination of LukAB and SpA* showed superior protection compared to the single antigens.

When minipigs were immunized with adjuvant only, high numbers of cfu were detected in the total muscle after challenge with the USA300 strain (GeoMean log₁₀ CFU/g of Muscle=5.48). A reduction in cfu in the muscle compared to the adjuvant group was observed after immunization with LukAB+adjuvant (GeoMean log₁₀ CFU/g of Muscle=3.37, P>0.05) and SpA*+adjuvant (GeoMean log₁₀ CFU/g of Muscle=2.84). A significant reduction of cfu in the muscle compared to the adjuvant group was detected when the animals were immunized with the combination of LukAB+SpA*+adjuvant (GeoMean log₁₀ CFU/g of Muscle=1.86, P=0.0198), suggesting that the combination of LukAB and SpA* showed superior protection against the USA300 strain compared to the single antigens (FIG. 30C). In the spleen, in general low levels of the CC8 USA300 strain were detected that were not significantly different between groups (FIG. 30D). Taken together, the surgical wound infection model data show that the vaccine combination comprising of LukAB and SpA* provided significant protection in the muscle of the minipig against challenges with two clinically relevant strains, ST398 and CC8 USA300.

Materials and Methods:

Antibody responses against LukAB and SpA measured by enzyme linked immunosorbent assay (ELISA): To measure IgG antibody levels against LukAB, 96-well maxisorp plates (Thermo Fisher Scientific) were coated with 1.0 μg/ml LukAB CC8 in PBS and incubated for 1 h at 2-8° C. After washing with PBS+0.05% Tween-20, plates were blocked with 2.5% skimmed milk, washed and serial 3-fold dilutions of serum prepared in diluent buffer (2.5% (w/v) skimmed milk powder in 1×PBS) starting at 1:100 were added to the wells. Plates were incubated for 1 hour at room temperature, washed and anti-Pig IgG-HRP secondary antibody (Sigma Aldrich) diluted 1:20,000 was added. After incubation at room temperature for 1 hour, plates were developed with TMB substrate (Leinco Technologies). The reaction was stopped by adding 1M sulphuric acid. Absorbance was read at 450 nm and EC₅₀ values were calculated using 4-PL (4 parameter logistic regression) curve fitting in Prism GraphPad V8.4.2.

To measure antibodies against SpA, 96-well maxisorp plates were coated with 0.25 μg/ml SpA* in PBS and incubated over night at 2-8° C. Secondary antibody was a 1:40,000 dilution of anti-Pig IgG-HRP in blocking buffer. The other steps were as described above for the measurement of anti-LukAB antibody responses. One-way ANOVA with Dunnett's multiple comparison test was performed to test statistical significance between geometric means of the vaccine groups vs the adjuvant group.

LukAB toxin neutralization assay. Cyto-Tox-One kit (Promega) was used to measure the release of lactate dehydrogenase (LDH) from cells with a damaged membrane. THP-1 cells were centrifuged and resuspended with RPMI to a density of 2×10⁶ cells/mL. 50 μL of cells were added to the 96 well culture plates containing serial 3-fold dilutions of serum. LukAB toxin CC8 was added to the test wells to a final concentration of 40 ng/mL. Lysis solution (Promega) was added to the lysis control wells. The plates were incubated for 2 hours at 37° C. in presence of 5% CO₂. The plates were centrifuged, 25 μL of the supernatant was transferred to a new plate and 25 μL CytoTox-ONE reagent (Promega) was added. Plates were incubated for 15 minutes at room temperature and stop solution (Promega) was added to the wells. Plates were read with the Biotek Synergy Neo 2 reader in monochromatic with an excitation wavelength of 560 and bandwidth of 5 nm and an emission wavelength of 590 and bandwidth of 10 nm. Gain is set at 120-130. One-way ANOVA with Dunnett's multiple comparison test was performed to test statistical significance between geometric means of the vaccine groups vs the adjuvant group.

Minipig Surgical Wound Infection Methods: Five to eight-month-old male Gottingen minipigs (Marshall Biosciences, North Rose, N.Y.) were group-housed and maintained on a 12-hour light/dark cycle with access to water ad libitum. On the morning of surgery, fasted minipigs were sedated, intubated, and placed under isoflurane anesthesia for the duration of the surgery. Surgery was performed on the left thigh whereby the muscle layer was exposed and a 5-mm bladeless trocar (Endopath® Xcel, Ethicon Endo-Surgery, Guaynabo, Puerto Rico) was advanced to the depth of the femur. A 20 μl inoculum (approx. 6 log₁₀ CFU/ml S. aureus) was injected into the wound (top of femur) via a 6-inch MILA spinal needle (Mila International, Inc., Florence, Ky.) through the trocar, which was then removed. The muscle was closed with a single silk suture, and the skin closed with absorbable PDS suture. Eight days later while under sedation, minipigs were euthanized with a barbiturate. Once death was confirmed, organs were processed separately for microbiology. Samples were homogenized in saline using a Bead Ruptor Elite (Omni International, Kennesaw, Ga., USA), then diluted and plated on TSA plates using an Autoplate 5000 Spiral Plater (Spiral Biotech, Norwood, Mass., USA). Plates were incubated 18-24 h at 37° C., then read on a QCount colony counter (Spiral Biotech, Norwood, Mass., USA).

One-way ANOVA with Dunnett's multiple comparison test was performed to test statistical significance between geometric means of multiple groups. All animal studies were reviewed and approved by the Janssen Spring House Institutional Animal Care and Use Committee and housed in an AAALAC-accredited facility.

Conclusion: A vaccine composition containing the antigens LukAB and SpA* with an adjuvant was shown herein to induce the generation of IgG against LukAB and SpA* in the minipig surgical wound infection model. The increase of anti-LukAB IgG antibody was associated with an increased neutralization of the cytotoxic activity of the LukAB toxin, indication that the induced IgG antibodies are functional. To test the efficacy of the vaccine composition, the ability of the vaccine to reduce the bacterial burden in the minipig surgical wound infection model was determined using two genetically different clinically relevant S. aureus strains. Immunization of minipigs with the LukAB+SpA*+adjuvant vaccine composition resulted in a significant reduction of the number of colony forming units in the muscle after challenge with both test strains. The vaccine composition also resulted in a significant reduction of cfu in the spleen with one of the test strain. Therefore, the S. aureus vaccine candidate containing LukAB and SpA toxoid mutants and an adjuvant effectively protected against deep-seated S. aureus infection and dissemination in a minipig surgical site infection model.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### REFERENCES

The following references, to the extent that they provide exemplary procedural or other details supplementary to those set forth herein, are specifically incorporated herein by reference.
33. Kim H K, Falugi F, Missiakas D, Schneewind O. 2016. Peptidoglycan-linked protein A promotes T-cell dependent antibody expansion during Staphylococcus aureus infection. Proc Natl Acad Sci USA 113:5718-5723.
37. Schulz D, Grumann D, Trube P, Pritchett-Corning K, Johnson S, Reppschlager K, Gumz J, Sundaramoorthy N, Michalik S, Berg S, van den Brandt J, Fister R, Monecke S, Uy B, Schmidt F, Broker B M, Wiles S, Holtfreter S. 2017. Laboratory mice are frequently colonized with Staphylococcus aureus and mount a systemic immune response—note of caution for in vivo infection experiments. Front Cell Infect Microbiol 7:152.
38. van Wamel W J, Rooijakkers S H, Ruyken M, van Kessel K P, van Strijp J A. 2006. The innate immune modulators staphylococcal complement inhibitor and chemotaxis inhibitory protein of Staphylococcus aureus are located on b-hemolysin-converting bacteriophages. J Bacteriol 188:1310-1315.
39. Jongerius I, Kohl J, Pandey M K, Ruyken M, van Kessel K P M, van Strijp J A G, Rooijakkers S H M. 2007. Staphylococcal complement evasion by various convertase-blocking molecules. J Exp Med 204:2461-2471.
40. Cheng A G, McAdow M, Kim H K, Bae T, Missiakas D M, Schneewind O. 2010. Contribution of coagulases towards Staphylococcus aureus disease and protective immunity. PLoS Pathog 6:e1001036.
41. McAdow M, Kim H K, DeDenta A C, Hendrickx A P A, Schneewind O, Missiakas D M. 2011. Preventing Staphylococcus aureus sepsis through the inhibition of its agglutination in blood. PLoS Pathog 7:e1002307.
42. McDevitt D, Francois P, Vaudaux P, Foster T J. 1994. Molecular characterization of the clumping factor (fibrinogen receptor) of Staphylococcus aureus. Mol Microbiol 11:237-248.
43. Baba T, Bae T, Schneewind O, Takeuchi F, Hiramatsu K. 2007. Genome sequence of Staphylococcus aureus strain Newman and comparative analysis of staphylococcal genomes. J Bacteriol 190:300-310.
44. Kim H K, Cheng A G, Kim H-Y, Missiakas D M, Schneewind O. 2010. Non-toxigenic protein A vaccine for methicillin-resistant Staphylococcus aureus infections. J Exp Med 207:1863-1870.
45. Cheng A G, Missiakas D M, Schneewind O. 2014. The giant protein Ebh is a cross wall determinant of Staphylococcus aureus cell size and complement resistance. J Bacteriol 196:971-981.
46. Becker S, Frankel M B, Schneewind O, Missiakas D M. 2014. Release of protein A from the cell wall envelope of Staphylococcus aureus. Proc Natl Acad Sci USA 111:1574-1579.
47. Goodyear C S, Silverman G J. 2004. Staphylococcal toxin induced preferential and prolonged in vivo deletion of innate-like B lymphocytes. Proc Natl Acad Sci USA 101:11392-11397.
48. Falugi F, Kim H K, Missiakas D M, Schneewind O. 2013. The role of protein A in the evasion of host adaptive immune responses by Staphylococcus aureus mBio 4:e00575-00513.
54. Lucero C A, Hageman J, Zell E R, Bulens S, Nadle J, Petit S, Gershman K, Ray S, Harrison L H, Lynfield R, Dumyati G, Townes J M, Schaffner W, Fridkin S K. 2009. Evaluating the potential public health impact of a Staphylococcus aureus vaccine through use of population-based surveillance for invasive methicillin-resistant S. aureus disease in the United States. Vaccine 27:5061-5068.
75. Pauli N T, Kim H K, Falugi F, Huang M, Dulac J, Dunand C H, Zheng N Y, Kaur K, Andrews S, Huang Y, Dedent A, Frank K, Chamot-Katsikas A, Schneewind O, Wilson P C. 2014. Staphylococcus aureus infection induces protein A-mediated immune evasion in humans. J Exp Med 211:2331-2339.
76. Kim H K, Falugi F, Thomer L, Missiakas D M, Schneewind O. 2015. Protein A suppresses immune responses during Staphylococcus aureus bloodstream infection in guinea pigs. mBio 6:e02369-02314.
84. Kim H K, Emolo C, DeDent A C, Falugi F, Missiakas D M, Schneewind O. 2012. Protein A-specific monoclonal antibodies and the prevention of Staphylococcus aureus disease in mice. Infect Immun 80:3460-3470.
85. DeLeo F R, Chambers H F. 2009. Waves of resistance: Staphylococcus aureus in the antibiotic era. Nat Rev Microbiol 7:629-641.
86. Harmsen D, Claus H, Witte W, Rothganger J, Claus H, Turnwald D, Vogel U. 2003. Typing of methicillin-resistant Staphylococcus aureus in a university hospital setting by using novel software for spa repeat determination and database management. J Clin Microbiol 41:5442-5448.
87. Bae T, Schneewind O. 2005. Allelic replacement in Staphylococcus aureus with inducible counter-selection. Plasmid 55:58-63.
88. Thomer L, Becker S, Emolo C, Quach A, Kim H K, Rauch S, Anderson M, Leblanc J F, Schneewind O, Faull K F, Missiakas D. 2014. N-acetylglucosaminylation of serine-aspartate repeat proteins promotes Staphylococcus aureus bloodstream infection. J Biol Chem 289:3478-3486.
102. Sun Y, Emolo C E, Holtfreter S, Wiles S, Kreiswirth B, Missiakas D, Schneewind O. 2018. Staphylococcal protein A is required for persistent colonization of mice with Staphylococcus aureus. J Bacteriol 200:e00735-17.
140. Gutafson G T, Stalenheim G, Forsgren A, Sjoquist J. 1968. Protein A from Staphylococcus aureus I V. Production of anaphylaxis-like cutaneous and systemic reactions in non-immunized guinea pigs. J Immunol 100:530-534.
142. Anderson A L, Sporici R, Lambris J, Larosa D, Levinson A I. 2006. Pathogenesis of B-cell superantigen-induced immune complex-mediated inflammation. Infect Immun 74:1196-1203.
145. Marone G, Tamburini M, Giudizi M G, Biagiotti R, Almerigogna F, Romagnani S. 1987. Mechanism of activation of human basophils by Staphylococcus aureus Cowan 1. Infect Immun 55:803-809.
146. Chen X, Sun Y, Missiakas D, Schneewind O. 2018. Staphylococcus aureus decolonization of mice with monoclonal antibody neutralizing protein A. J Infect Dis in press.
154. Deisenhofer J. 1981. Crystallographic refinement and atomic models of a human Fc fragment and its complex with fragment B of protein A from Staphylococcus aureus at 2.9- and 2.8-A resolution. Biochemistry 20:2361-2370.
155. Fisher M M. 1986. Clinical observations on the pathophysiology and treatment of anaphylactic cardiovascular collapse. Anaesth Intensive Care 14:17-21.
156. Korhonen H, Fisslthaler B, Moers A, Wirth A, Habermehl D, Wieland T, Schutz G, Wettschureck N, Fleming I, Offermanns S. 2009. Anaphylactic shock depends on endothelial Gq/G11. J Exp Med 206:411-420.
157. Nakamura Y, Oscherwitz J, Cease K B, Munoz-Planillo R, Hasegawa M, McGavin M J, Otto M, Inohara N, Nunez G. 2013. Staphylococcus 6-toxin promotes allergic skin disease by inducing mass cell degranulation. Nature 503 review:397-401.
158. Kitamura D, Roes J, Kihn R, Rajewsky K. 1991. A B cell-deficient mouse by targeted disruption of the membrane exon of the immunoglobulin mu chain gene. Nature 350:423-426.

1. An immunogenic composition comprising:
(a) a Staphylococcus aureus protein A (SpA) variant polypeptide, wherein the SpA variant polypeptide comprises at least one SpAA, B, C, D, or E domain; and
(b) a mutant staphylococcal leukocidin subunit polypeptide comprising:
(i) a mutant LukA polypeptide,
(ii) a mutant LukB polypeptide, and/or
(iii) a mutant LukAB dimer polypeptide,
wherein (i), (ii), and/or (iii) have one or more amino acid substitutions, deletions, or a combination thereof,
such that the ability of the mutant LukA, LukB, and/or LukAB polypeptides to form pores in the surface of eukaryotic cells is disrupted, thereby reducing the toxicity of the mutant LukA and/or LukB polypeptide or the mutant LukAB dimer polypeptide relative to the corresponding wild-type LukA and/or LukB polypeptide or LukAB dimer polypeptide.

2. The immunogenic composition of claim 1, wherein the SpA variant polypeptide has at least one amino acid substitution that disrupts Fc binding and at least a second amino acid substitution that disrupts V_{H}3 binding.

3. The immunogenic composition of claim 1, wherein the SpA variant polypeptide comprises a SpA D domain and has an amino acid sequence having at least 90% identity to the amino acid sequence of SEQ ID NO:58.

4. The immunogenic composition of claim 3, wherein the SpA variant polypeptide has one or more amino acid substitutions at amino acid position 9 or 10 of SEQ ID NO:58.

5. The immunogenic composition of claim 3, wherein the SpA variant polypeptide further comprises a SpA E, A, B, or C domain.

6. The immunogenic composition of claim 5, wherein the SpA variant polypeptide comprises a SpA E, A, B, and C domain and has an amino acid sequence having at least 90% identity to the amino acid sequence of SEQ ID NO:54.

7. The immunogenic composition of claim 5, wherein each SpA E, A, B, and C domain has one or more amino acid substitutions at positions corresponding to amino acid positions 9 and 10 of SEQ ID NO:58.

8. The immunogenic composition of claim 4, wherein the amino acid substitution is a lysine residue for a glutamine residue.

9. The immunogenic composition of claim 1, wherein the SpA variant polypeptide comprises at least one SpAA, B, C, D, or E domain, and wherein the at least one domain has (i) lysine substitutions for glutamine residues corresponding to positions 9 and 10 in the SpA D domain and (ii) a glutamate substitution corresponding to position 33 in the SpA D domain, wherein the polypeptide does not, relative to a negative control, detectably crosslink IgG and IgE in blood or activate basophils.

10. The immunogenic composition of claim 9, wherein the SpA variant polypeptide has a reduced K_{A} binding affinity for V_{H}3 from human IgG as compared to a SpA variant polypeptide (SpA_{KKAA}) comprising lysine substitutions for glutamine residues in each SpAA-E domain corresponding to positions 9 and 10 in the SpA D domain and alanine substitutions for aspartic acid residues in each SpA A-E domain corresponding to positions 36 and 37 of SpA D domain.

11. The immunogenic composition of claim 10, wherein the SpA variant polypeptide has a K_{A} binding affinity for V_{H}3 from human IgG that is reduced by at least 2-fold as compared to SpA_{KKAA}.

12. The immunogenic composition of claim 10, wherein the SpA variant polypeptide has a K_{A} binding affinity for V_{H}3 from human IgG of less than 1×10⁵ M¹.

13. The immunogenic composition of claim 1, wherein the SpA variant polypeptide does not have substitutions in any of the SpA A, B, C, D, or E domains corresponding to amino acid positions 36 and 37 in the SpA D domain.

14. The immunogenic composition of claim 9, wherein the only substitutions in the SpA variant polypeptide are (i) and (ii).

15. The immunogenic composition of claim 1, wherein the mutant LukA polypeptide comprises an amino acid sequence having at least 90% sequence identity to any one of SEQ ID NOs:1-28.

16. The immunogenic composition of claim 15, wherein the mutant LukA polypeptide comprises a deletion of the amino acid residues corresponding to amino acid positions 342-351 of any one of SEQ ID NOs:1-14 and at amino acid positions 315-324 of any one of SEQ ID NOs:15-28.

17. The immunogenic composition of claim 1, wherein the mutant LukB polypeptide comprises an amino acid sequence having at least 80% sequence identity to any of SEQ ID NO:29-53.

18. The immunogenic composition of claim 1, wherein the mutant LukAB dimer polypeptide comprises a mutant LukA polypeptide having a deletion of the amino acid residues corresponding to positions 315-324 of SEQ ID NO:16; and a LukB polypeptide comprising the amino acid sequence of SEQ ID NO:53.

19. The immunogenic composition of claim 1, further comprising an adjuvant.

20-26. (canceled)

27. One or more isolated nucleic acids encoding a Staphylococcus aureus protein A (SpA) variant polypeptide and a mutant Luk A polypeptide, a mutant Luk B polypeptide, or a mutant LukAB dimer polypeptide according to claim 1.

28. A vector comprising the isolated nucleic acid of claim 27.

29. An isolated host cell comprising the vector of claim 28.

30. A method for treating or preventing a Staphylococcus infection, for eliciting an immune response to a Staphylococcus bacterium, or for decolonizing or preventing colonization or recolonization of a Staphylococcus bacterium in a subject in need thereof, the method comprising administering to the subject in need thereof an effective amount of the immunogenic composition of claim 1.

31-32. (canceled)
